(19) United States
(12) Patent Application Publication
(10) Pub. No.: US 2008/0277265 A1
(43) Pub. Date: Nov. 13, 2008

(54) GAS REFORMULATION SYSTEM COMPRISING MEANS TO OPTIMIZE THE EFFECTIVENESS OF GAS CONVERSION

(75) Inventors: Andreas TSANGARIS, Ottawa (CA); Marc BACON, Saint-Constant (CA)

Correspondence Address: CHRISTOPHER & WEISBERG, P.A., 200 EAST LAS OLAS BOULEVARD, SUITE 2040, FORT LAUDERDALE, FL, 33301, US

(73) Assignee: PLASCO ENERGY GROUP, INC., Kanata (CA)

(21) Appl. No.: 12/119,413

(22) Filed: May 12, 2008

Related U.S. Application Data
(60) Provisional application No. 60/917,410, filed on May 11, 2007.
(60) Provisional application No. 60/986,213, filed on Nov. 7, 2007.
(60) Provisional application No. 60/986,212, filed on Nov. 7, 2007.
(60) Provisional application No. 61/042,571, filed on Apr. 4, 2008.

(30) Foreign Application Priority Data
Feb. 27, 2008 (CA) PCT/CA08/00355

(51) Int. Cl. C07C 1/00; G01N 27/26

(52) U.S. Cl. 204/157.15; 204/422

(57) ABSTRACT

This invention provides a system and method for efficient reformulation of an initial gas with associated characteristics into an output gas with desired characteristic parameters, within a substantially sealed, contained, and controlled environment. The gas reformulating system uses a gas energizing field to disassociate the initial gas molecules and molecules of injected process additives of appropriate types and amounts, into their constituents that then recombine to form the output gas with the desired parameters. The gas reformulating system further comprises a control system that regulates the process and thereby enables the process to be optimized. The gas energizing field may be provided at least partly by hydrogen burners or plasma torches.

### CROSS-REFERENCE TO RELATED APPLICATIONS

This application claims benefit of priority under 35 U.S.C. § 119(e) from U.S. Provisional Application Ser. No. 60/917,410, filed May 11, 2007. This application also claims benefit of priority under 35 U.S.C. § 119(e) from U.S. Provisional Application Ser. No. 60/986,213, filed Nov. 7, 2007. This application also claims benefit of priority under 35 U.S.C. §119(e) from U.S. Provisional Application Ser. No. 60/986,212, filed Nov. 7, 2007. This application also claims the benefit of priority to International Patent Application No. PCT/CA08/00355, filed Feb. 27, 2008. This application also claims benefit of priority under 35 U.S.C. §119(e) from U.S. Provisional Application Ser. No. 61/042,571, filed Apr. 4, 2008. The contents of all of the aforementioned applications are hereby expressly incorporated by reference in their entirety and for all purposes.

### FIELD OF THE INVENTION

This invention pertains to the field of gas reformulation. In particular, it relates a gas reformulation system comprising means to optimize the effectiveness of gas conversion.

### BACKGROUND OF THE INVENTION

Off-gas (syngas) is produced from a variety of material conversion processes such as gasification, plasma gasification and/or plasma melting etc. These gases may be utilized in appropriate downstream applications (e.g. power generation, industrial synthesis of chemicals and liquid fuels), stored for later use or flared off. In some cases, there is interest in reformulating the gas that is produced in order to improve the chemical composition for efficient utilization in downstream applications.

In gasification processes, carbonaceous feedstock is fed into a gasifier along with a controlled and/or limited amount of oxygen and sometimes steam, to produce a raw gas. The off-gas from a gasification process depends on the feedstock composition and may contain H₂O, H₂, N₂, O₂, CO₂, CO, CH₄, H₂S, NH₃, C₂H₆, and other hydrocarbons such as acetylenes, olefins, aromatics, phenols, and tars. Useful feedstock for gasification include municipal waste, waste produced by industrial activity and biomedical waste, sewage, sludge, coal, heavy oils, petroleum coke, heavy refinery residuals, refinery wastes, hydrocarbon contaminated soils, biomass, agricultural wastes, tires, and other hazardous waste.

Factors that affect the quality of the gas produced in gasification processes include: feedstock characteristics such as particle size; gasifier heating rate; residence time; plant configuration including whether it employs a dry or slurry feed system, feedstock-reactant flow geometry, design of ash or slag mineral removal system; whether it uses a direct or indirect heat generation and transfer method; and the syngas cleanup system.

Some gasification facilities employ gas treatment systems to convert the gas into a more acceptable gas composition prior to cooling and cleaning through a gas quality conditioning system. The treated gas may undergo further treatment steps for the removal of unwanted compounds such as metals, sulfur compounds and fine particulates. For example, dry filtration systems and wet scrubbers can be used to remove particulate matter and acid gases.

Plasma has been used for two predominant sources of energy by industry: one as a source of intense heat, and secondly as a source of free electrons that can be used to initiate and drive many chemical processes requiring the dissociation of molecules into (reactive) dissociation fragments. The electron impact can excite any dissociative state of a molecule and reduce it to fragments, which is a key mechanism by which radicals and molecular fragments are produced in many environments.

Plasma is a luminous gas that is at least partially ionized, and is made up of excited gaseous substances including electrons and ions. Plasma can be produced with many gases, thus giving excellent control over chemical reactions in the plasma as the working gas may be neutral (e.g. argon, helium, neon), reductive (e.g. hydrogen, methane, ammonia, carbon monoxide), or oxidative (e.g. oxygen, carbon dioxide).

Different plasmas are classified according to their temperature and density. The term “plasma density” by itself usually refers to the electron density, that is, the number of free electrons per unit volume. The degree of ionization of a plasma is the proportion of atoms which have lost (or gained) electrons, and is controlled mostly by the temperature.

Plasma temperature is commonly measured in kelvins or electronvolts, and is an informal measure of the average thermal kinetic energy per particle. Because of the large difference in mass, the electrons come to thermodynamic equilibrium among themselves much faster than they come into equilibrium with the ions or neutral atoms. For this reason the “ion temperature” may be very different from (usually lower than) the “electron temperature.” Based on the relative temperatures of the electrons, ions, and neutrals, plasmas are classified as “thermal” or “non-thermal.” Thermal plasmas have electrons and the heavy particles at the same temperature, i.e., they are in thermal equilibrium with each other. Non-thermal plasmas on the other hand have the ions and neutrals at a much lower temperature whereas electrons are much “hotter.”

Non-thermal, low-temperature plasmas are known in the art to destroy relatively low concentrations of volatile organic compounds at atmospheric pressure and are particularly attractive for treatment of low-level waste concentrations and for dealing with compounds that resist treatment by standard chemical means. These low-temperature plasma processing techniques generally involve either high energy electron-beam irradiation or electrical discharge methods such as pulsed corona, dielectric barrier, capillary, hollow cathode, surface, and packed-bed corona discharge. All of these techniques rely upon the fact that electrical energy can produce electrons with much higher average kinetic energies than the surrounding gas-phase ions and molecules. These energetic electrons can interact with the background gas to produce highly reactive species (i.e., radicals, anions, cations, and secondary electrons) that will preferentially destroy pollutants.

In the field of waste management, plasma torches have been used as a source of heat to drive the gasification, melting and destruction of hazardous waste, by converting it to an off-gas (i.e., syngas) and melting the residue which mostly comprises inorganic substances into slag. Some plasma gasification systems use plasma torches not only to drive the gasification process but also to treat the raw off-gas in the gasification chamber by converting, reconstituting, or reforming longer chain volatiles into smaller molecules with or without the addition of other inputs or reactants.

Plasma sources have also been used as a source of active species. These active species have been used to initiate and drive the conversion of hazardous gaseous molecules into less toxic species. One example is provided by U.S. Pat. No. 6,810,821 which describes a cyclonic oxidizer designed for reducing carbon black/soot present in the off-gas from a graphite electrode plasma arc furnace. The cyclonic oxidizer uses a plasma torch to ionize a working gas comprising carbon dioxide and oxygen mixture that excludes nitrogen. When the gas mixture is ionized in the plasma arc zone, the carbon dioxide is converted to carbon monoxide and atomic oxygen, which is very reactive. The cyclonic oxidizer chamber receives the off-gas tangentially near its upstream end at very high velocity, thereby creating a cyclonic condition within the cyclonic oxidizer. Combining the presence of reactive atomic oxygen and the enhanced turbulent environment in the cyclonic oxidizer, carbon black/soot and the fugitive toxic materials in the by-product gas can be effectively converted and destroyed.

U.S. Pat. No. 6,810,821 also teaches that additional oxidizing agents are provided by the injection of atomized oxygen and steam that are atomized by high temperature resistance atomizing nozzles and injected into the chamber as oxidizing agents. The oxidation reaction efficiency is increased by the intense internal mixing between the by-product gas and injected atomized oxygen and steam caused by the vigor of the cyclonic action within the cyclonic oxidizer. With low heating value wastes, the cyclonic oxidizer converts the by-product gas completely to water and carbon dioxide. With high heating value wastes, the final by-product gas can be a high quality combustible synthetic gas for electricity generation. Although this cyclonic oxider, can treat (i.e., clean) the off-gas by oxidizing the contaminates, it is not designed to reformulate the gas into a product gas of designed chemical composition. It does not use the plasma torch to create a gas reformulation zone that can be used to reformulate the off-gas into a gas of a defined composition.

Another example is provided by U.S. Pat. No. 6,030,506 which describes a method and apparatus for the delivery of exogenous non-thermal plasma activated species to a subject fluid comprising: (a) creating activated species in an energizing means; and (b) introducing the activated species into a subject fluid by high speed injection means. This invention addresses air pollution control as well as providing an apparatus and method of performing large scale chemistry for bleaching, enhancing chemical reactions, and pollution removal.

U.S. patent application Ser. No. 11/745,414 provides the first example of a gas reformulating system wherein the positioning of plasma torches within the system provides reactive fields in front of each torch whereby the off-gas can be reformulated. The positioning of these plasma torches and air jets is designed to optimize the flow patterns and residence time of the gas in the chamber.

The aforementioned systems do not optimize the energetic mechanisms and the overall effectiveness of reformulating the majority of raw syngas to gas of a designed chemical composition. Commercial facilities seeking to convert carbonaceous feedstock to energy such as electricity in the most overall cost efficient manner require systems for the effective conversion of the syngas to gas of a composition designed for downstream applications. Accordingly, it would be a significant advancement in the art to provide a gas reformulation system that optimizes the overall effectiveness of the process, and/or the steps comprising the overall process of converting an initial gas to a gas of a defined composition.

### SUMMARY OF THE INVENTION

The invention provides a system that incorporates one or more energy sources that initiate the process of reformulation of a gas by initiating the dissociation of molecules into reactive dissociation fragments (intermediates). The energy source(s) is combined with Gas Manipulators designed to optimize the effectiveness of the gas reformulation process by optimizing energy transference throughout the process of gas reformulation in addition to optimizing the amount of gas that is reformulated relative to the amount of gas that is input into the system (the Gas Reformulation Ratio).

An object of the invention is to provide a gas reformulation system comprising means to optimize the effectiveness of gas conversion. In accordance with an aspect of the invention, there is provided a system for reformulating an initial gas into a reformulated gas having designed characteristics, comprising a means for sensing at least one characteristic of the initial gas; means for modifying process inputs for reformulation based on the at least one characteristic of the initial gas, and on the designed characteristics of the reformulated gas; means for applying one or more energy sources sufficient to reformulate a substantial majority of the gaseous molecules of the initial gas into the reformulated gas; means for promoting the reformulation; means for stabilizing the reformulated gas; and a control system.

In accordance with another aspect of the invention, there is provided a process for reformulating an initial gas into a reformulated gas having desired characteristics, comprising one or more of the following steps sensing at least one characteristic of the initial gas; modifying process inputs for reformulation based on the sensed characteristics of the initial gas, and on the desired characteristics of the output gas; applying a gas energizing field sufficient to reformulate the majority of the gaseous molecules into their constituents; promoting efficient process acceleration for the reformulation of the constituents into a reformulated gas of designed characteristics; promoting the de-energization and stabilization of the newly formed molecules to maintain the designed characteristics; and managing the efficient conversion of the initial gas to the output gas.

In accordance with another aspect of the invention, there is provided a system for the reformulation of gas comprising one or more energy sources for the initiation of gas reformulation processes; and one or more Gas Manipulators for the optimization of energy transference throughout the process of gas reformulation; wherein the one or more energy sources and the one or more Gas manipulators are integrated to optimize the Gas Reformulation Ratio.

In accordance with another aspect of the invention, there is provided a gas reformulation system comprising one or more gas reformulating zones; one or more gas stabilizing zones; comprises a control system that regulates the overall process; optionally one or more gas additive zones, and/or optionally one or more gas cleaning zones, wherein the zones of the system are arranged and controlled in such a manner that the majority of the initial gas is reformulated into gas of a designed composition.

In accordance with another aspect of the invention, there is provided a method for reformulating of an initial gas into a reformulated gas, comprising the steps of delivering the initial gas to a gas reformulating chamber; mixing the input gas with at least one process additive to create preformulated gas; exposing the preformulated gas to a gas energizing field thereby disassociating molecules within the gas into their constituent elements; reforming the constituent elements into molecular species of a designed chemical composition and thereby producing the reformulated gas; and removing the reformulated gas from the chamber.

In accordance with another aspect of the invention, there is provided a system for reformulating of an initial gas into a reformulated gas comprising one or more refractory-lined chambers comprising one or more inputs for receiving the initial gas; one or more outputs for releasing the reformulated gas; one or more process additive inputs in fluid communication with the chamber; one or more gas manipulators located in the one or more chambers; means to create a gas energizing field within the one or more chambers.

In particular, this system has been designed to optimize the transfer of energy from one or more sources to gas of an initial chemical composition (preformulated gas) and throughout the reformulation process such that the gas reformulates into gas of a designed chemical composition in an effective manner. This system comprises design strategies embodied within the Gas Manipulators that function to facilitate the speed, efficiency and thoroughness of the reformulation reactions as the gas passes through the gas reformulation chamber, to minimize the amount of energy required overall to reformulate gas, and to maximize the percentage of gas reformulated into gas of a designed chemical composition.

Accordingly, the gas reformulation system comprises one or more “gas reformulating zones,” and one or more “gas stabilizing zones.” The system can optionally further comprise one or more “gas additive zones,” generally located upstream of a gas reformulating zone, with or without means to accomplish mixing of the gas with the additives, mixing is generally accomplished by increasing the turbulence within the gas, and/or one or more “gas cleaning zones,” generally located downstream of a gas stabilizing zone. A gas stabilizing zone optionally comprises heat transfer means to capture heat from the gas as it cools. The zones of the system are arranged and controlled in such a manner that the majority of the initial gas is reformulated into gas of a designed composition after passing through the system of this invention. The gas reformulating system further comprises a control system that regulates the overall process.

### BRIEF DESCRIPTION OF THE FIGURES

FIGS. 1 to 77 show various embodiments of the invention and/or its constituent parts.

FIGS. 1, 2A and 2B show the various zones of the gas reformulating system. The dotted lines show zones that are optional. The gases may undergo processing in a serial cascade of the zones or in parallel array as depicted in FIGS. 2BA and 2BB.

FIG. 3 is a schematic of the gas reformulating system according to an embodiment of the invention.

FIG. 4 is a schematic of one embodiment of a gas reformulating system of the invention coupled to a gasifier.

FIG. 5 is a schematic of one embodiment of a gas reformulating system of the invention coupled to two gasifiers.

FIG. 6 is a schematic of one embodiment of the gas reformulating chamber of the invention coupled to two gasifiers, through a common initial gas inlet.

FIGS. 7, 8, 13 and 14 show the following types of gas energizing sources: hydrogen burner, radio frequency (RF) and microwave plasma, laser plasma, corona plasma.

FIG. 9 shows the following types of plasma sources: non-transferred arc torch, transferred arc torch, inductively coupled plasma torch, microwave plasma torch.

FIG. 12 show a hydrogen burner.

FIGS. 10 and 11 illustrate the use of an inductively coupled plasma torch, microwave plasma torch and a hydrogen burner in a gas reformulating system, in accordance with various embodiments of the invention.

FIG. 15 shows various embodiments of gas reformulating channels.

FIG. 16 shows various embodiments of gas reformulating channels.

FIG. 17 shows various embodiments of gas reformulating channels.

FIG. 18 shows various embodiments of gas reformulating channels.

FIG. 19 shows a gas reformulating channel using a mixer device.

FIGS. 20A-B show the use of constrictions in the gas reformulating chamber for enhancing gas mixing, in accordance with two embodiments of the invention.

FIGS. 21A-B, 22 and 23 show various gas reformulating chamber designs.

FIG. 24 shows various embodiments of the gas reformulation system wherein the gas stream is separated into smaller streams which undergo reformulation in parallel.

FIG. 25 shows various arrangements of the gas energizing sources vis-à-vis the initial gas stream.

FIGS. 26A-C show different shapes of flow restrictors inserted into a gas reformulating chamber, in accordance with various embodiments of the invention.

FIGS. 27A-B and 42 show flow restrictors that extend for substantially whole length of the gas reformulating chamber, in accordance with three embodiments of the invention.

FIGS. 28A-B show the three dimensional view of gas reformulating chambers equipped with flow restrictors that extend for substantially whole length of the chamber, in accordance with two embodiments of the invention.

FIG. 29A-G show different embodiments of the flow restrictors.

FIG. 30A shows a rotational shaft with multiple disks, in accordance with one embodiment of the invention. FIG. 30B show different disk structures that can be used with the rotational shaft for enhanced interaction of the gas with energizing fields.

FIG. 31A-C show different rotational methods for the shaft and the disks, in accordance with various embodiments of the invention.

FIGS. 32 and 33 show the use of deflectors and Coanda-effect deflectors respectively for directing the gas energizing fields, in accordance with two embodiments of the invention.

FIG. 34A-B show the use of one or more air nozzles for active control of the spatial distribution of the plasma plume, in accordance with two embodiments of the invention.

FIGS. 35A-D show the use of different deflectors for redirection of the plasma plumes within the gas reformulating chamber.

FIGS. 36A-D show the use of asymmetric rotating shaft objects deflectors, in accordance with various embodiments of the invention.

FIG. 37 is a schematic of a portion of the gas reformulating system detailing the torch mounting system and according to an embodiment of the invention.

FIG. 38A shows a gas energizing source positioned to direct the gas energizing field counter-current to the flow of the gas stream, in accordance with one embodiment of the invention. FIG. 38B shows the embodiment of FIG. 38B with the gas entering near the top and exiting towards the bottom. FIG. 38C is a schematic illustrating the orientation of the inlets and plasma torches of one embodiment.

FIGS. 39 and 40 show various arrangements of the gas energizing sources vis-à-vis the gas reformulating chamber and the input gas stream.

FIG. 41 illustrates arrangements of baffles in the gas reformulating chamber. FIG. 41A illustrates air-flow within the gas reformulating chamber comprising bridge wall baffles. FIG. 41B illustrates air-flow within the gas reformulating chamber comprising turbulator or choke ring baffles.

FIGS. 43A-B show the inclusion of turbulence zones for enhanced reformulation. FIG. 43C show examples of turbulence generators.

FIG. 44 shows the gas to be reformulated entering tangentially into the reformation reactor creating a swirl which is treated by the plasma torches and the gas manipulator.

FIGS. 45 and 46 show exemplary means for generating turbulence.

FIG. 47 is a diagram illustrating air-flow out of a Type A nozzle.

FIG. 48 is a diagram illustrating air-flow out of a Type B nozzle.

FIGS. 49 and 50 show a fixed bed of char used as a catalyst in the reformulation chamber.

FIG. 51 shows a gasifier in combination with a gas reformulating chamber, wherein the char created in the gasifier leads to catalytic cracking.

FIGS. 52 to 54 show various configurations for combining catalyst beds and energizing fields for reformulation of gas generated within a gasifier.

FIGS. 55 to 57 show various positions where catalytic beds may be placed within a gas reformulating chamber, in accordance with one embodiment of the invention.

FIGS. 58 & 59 are related to the heat exchange systems used within the stabilizing zone of the gas reformulating system, in accordance with one embodiment of the invention.

FIG. 60A is a schematic of one embodiment of the gas reformulating chamber. FIG. 60B is a cross sectional view of the gas reformulating chamber of FIG. 60A detailing the refractory supports.

FIGS. 61 to 64 show various configurations of gas reformulating chambers, gasifiers and carbon converters.

FIG. 65 shows a gasifier which may be linked to the gas reformulating system of the invention.

FIGS. 66 to 68, 74 and 77 show various views of an exemplary gas manipulator designed to be retrofitted to a cylindrical gas reformulating chamber.

FIGS. 69, 70, 72, 73, 75, 76 show various views of the exemplary gas manipulator of FIG. 66 as installed in the cylindrical gas reformulating chamber.

FIG. 71 shows a top view of the gas reformulating chamber without the exemplary gas manipulator of FIG. 66.

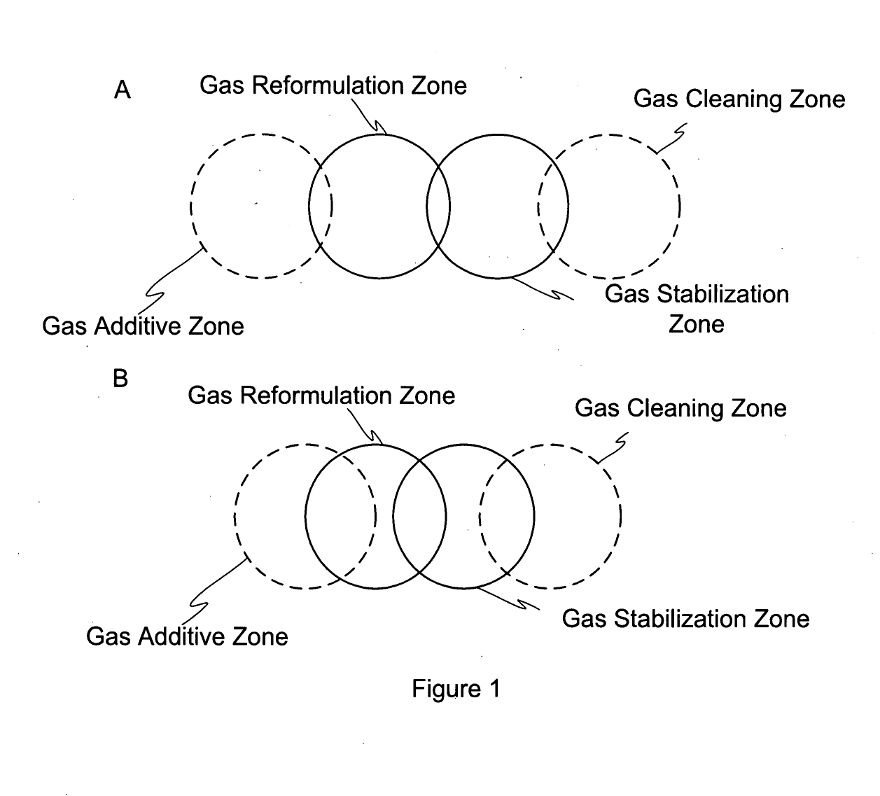

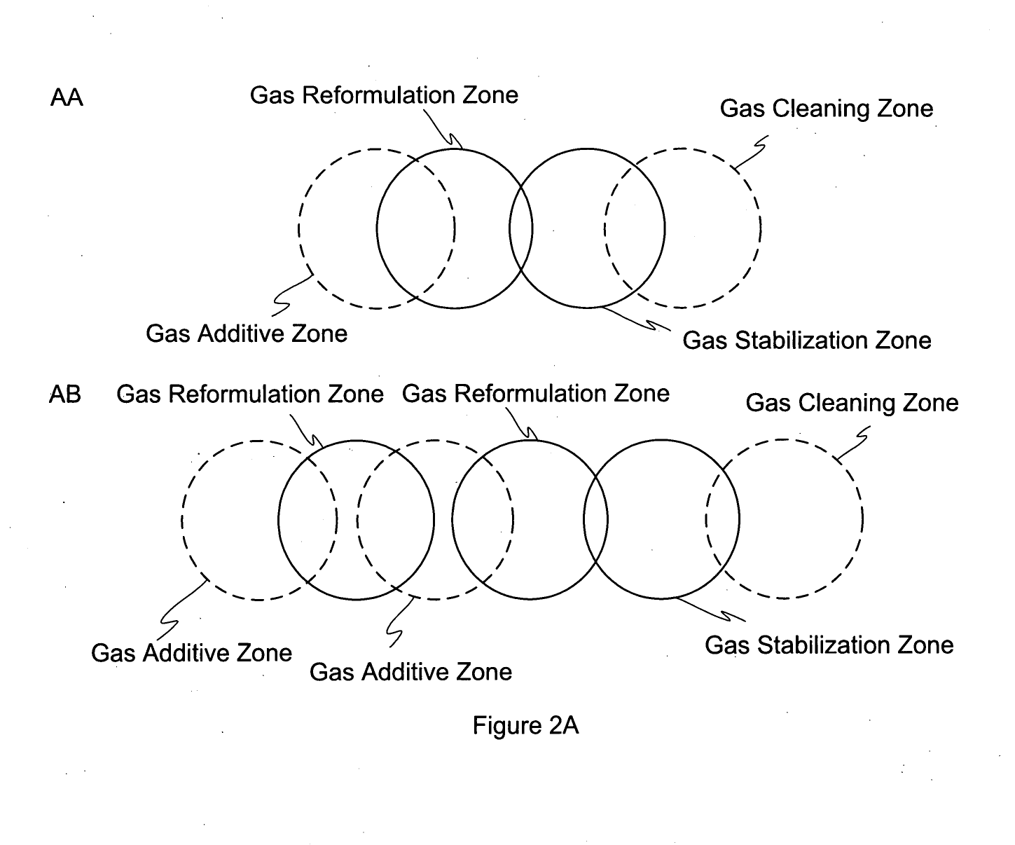

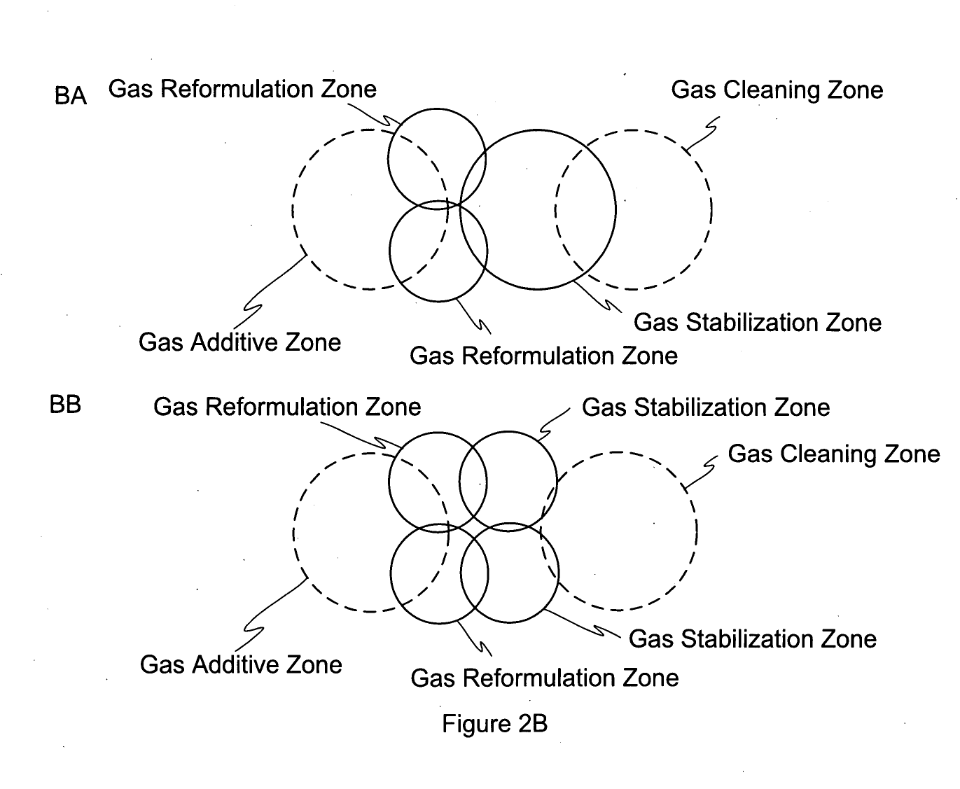

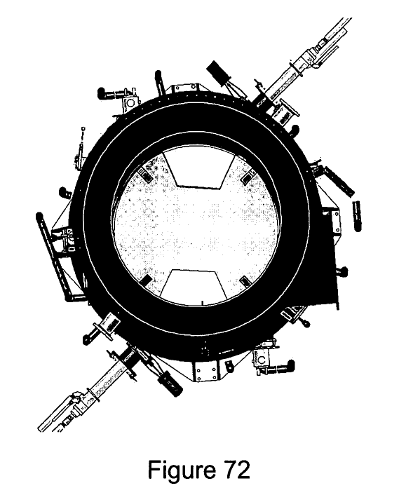

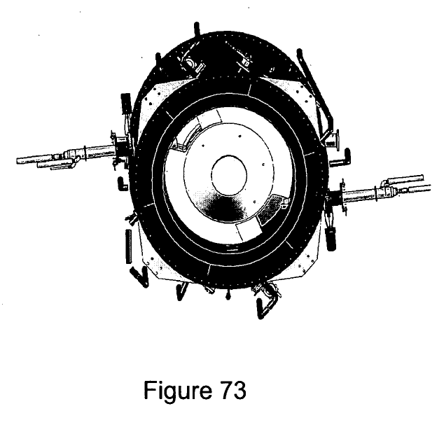

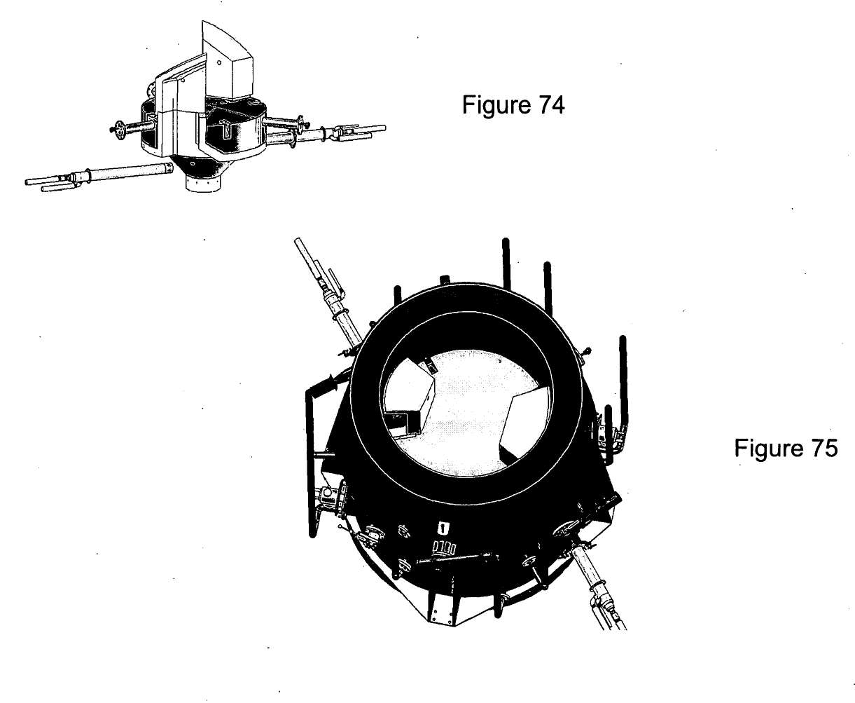

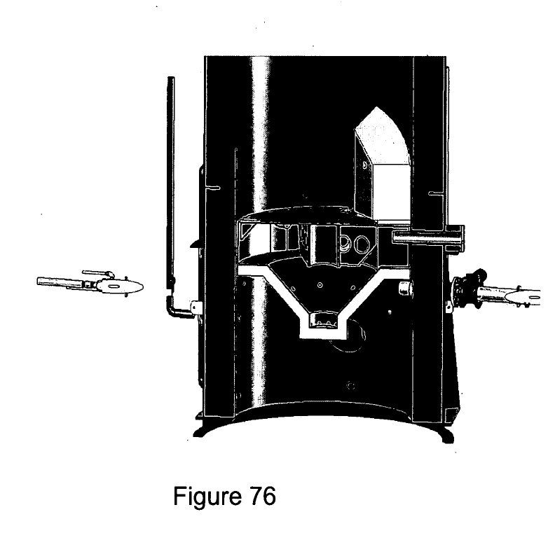

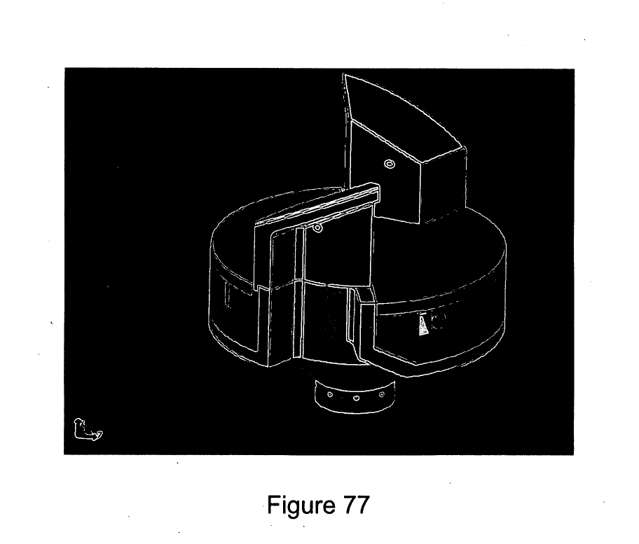

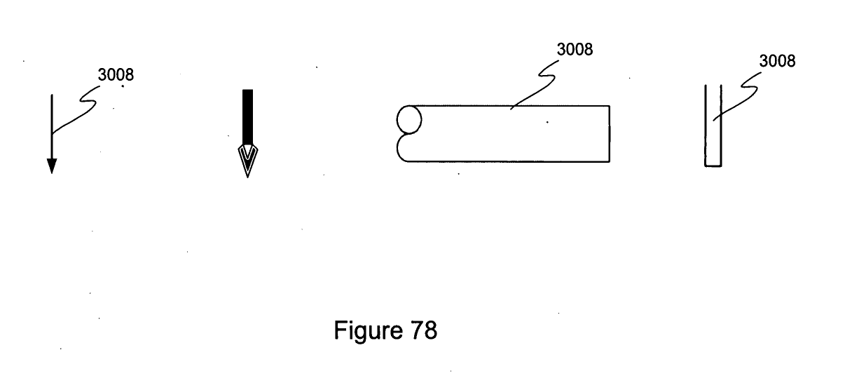

FIG. 78 show various representations for the gas energizing sources as used in the FIGS. 1 to 77. All representations are equivalent and can be used to indicate any of the gas energizing sources specifically indicated herein, or as would be known to a worker skilled in the art.

### DETAILED DESCRIPTION OF THE INVENTION

Unless defined otherwise, all technical and scientific terms used herein have the same meaning as commonly understood by one of ordinary skill in the art to which this invention belongs.

As used herein, the term “about” refers to a ±10% variation from the nominal value. It is to be understood that such a variation is always included in any given value provided herein, whether or not it is specifically referred to.

The term, “reactive species,” refers to energetic species formed throughout the reformulation process. Non-limiting examples include free electrons generated by an energy source such as plasma, or radicals or dissociated intermediates (induced intermediates) that are created in the off-gas (e.g., syngas) that transfer energy to other molecules and/or dissociated intermediates/fragments of the preformulated gas (“preformulated molecules”) enabling them to reformulate into a chemical composition of designed specifications. One skilled in the art appreciates that as the energy transference process continues, some of the preformulated molecules will in turn become reactive species, transferring their acquired energy to other molecules in the gas reformulating zone.

The term, “raw off-gas,” refers to the gas that comes off the feedstock throughout the process of converting it to slag. This type and quality of gas is often referred to in the industry as “syngas.”

The term, “partially processed raw off-gas,” refers to the raw off-gas (raw syngas) that has been somehow processed due to the conditions, such as intense heat or reactive species, produced in a gasification system such as a plasma melting system, designed for the destruction of waste and conversion into gas and slag. Such processing can include exposure of the raw off-gas to plasma or other energy sources.

The term, “initial gas,” refers to the gas to be reformulated into a chemical composition designed for one or more downstream applications. It includes raw off-gas (raw syngas) and/or partially processed raw off-gas.

The term, “preformulated gas,” is used to denote gas as it enters a gas reformulating zone. This gas comprises the initial gas in addition to any optional process additives that have been added to adjust the chemical composition of the gas prior to reformulating it into a designed chemical composition. For example, if the gas requires increased levels of hydrogen, steam may be added as a process additive upstream of a gas reformulation zone, such that the reformulating gas will contain sufficient amounts of hydrogen species to provide for the proper chemical composition of the final reformulated gas product. If no optional process additives have been added “preformulated gas” has the same composition as “initial gas”.

The term, “reformulated gas,” refers to the gas that exits the Gas Reformulation System.

The term, “Gas Reformulation Ratio,” is used to describe the amount of gas that is reformulated relative the amount of gas that is input into the system. It can be described by the formula:

$$\frac{Amount of Reformulated Gas}{Amount of Preformulated Gas} \times 100 = \% of gas reformulated$$

Alternatively, and especially if no process additive gases are used, it can be described by the formula:

$$\frac{Amount of Reformulated Gas}{Amount of Initial Gas} \times 100 = \% of gas reformulated$$

The Gas Reformulation Ratio can be assessed directly or indirectly. Indirect assessment of the gas reformulation ratio can made by comparing downstream energy production of reformulated gas and preformulated gas. Downstream energy production is reflective of percent gas reformulated. An increase in downstream energy production is indicative of increased percent gas reformulated.

The term, “Gas Manipulators,” denotes the features incorporated into the system of this invention that function to facilitate the process of gas reformulation.

The terms ‘carbonaceous feedstock’ and ‘feedstock’, as used interchangeably herein, are defined to refer to carbonaceous material that can be used in the gasification process. Examples of suitable feedstock include, but are not limited to, hazardous and non-hazardous waste materials, including municipal wastes; wastes produced by industrial activity; biomedical wastes; carbonaceous material inappropriate for recycling, including non-recyclable plastics; sewage sludge; coal; heavy oils; petroleum coke; bitumen; heavy refinery residuals; refinery wastes; hydrocarbon contaminated solids; biomass; agricultural wastes; municipal solid waste; hazardous waste and industrial waste. Examples of biomass useful for gasification include, but are not limited to, waste wood; fresh wood; remains from fruit, vegetable and grain processing; paper mill residues; straw; grass, and manure.

The term, “gas energizing sources,” refers to any source of energy known to one skilled in the art that could be used to impart energy to the preformulated gas, enabling it to reformulate into gas of a defined composition. Examples include, without limitation, plasma generating sources, radiation sources, hydrogen burners, electron beam guns, etc

The term, “gas energizing field,” is used to denote the field effect produced by one or more of the gas energizing sources used within this system to provide the energy to the gas that is required for the reformulation process to occur. For example, the gas energizing field that is created by a plasma torch will exhibit a three-dimensional space that will vary with torch power, working gas composition, torch position, torch orientation, etc.

As used herein, the term “sensing element” is used in the broadest sense to describe the aspect of any element related to the gas reformulation system that is configured to sense, detect, read, monitor, etc. one or more characteristics, parameters, and/or information of the system, inputs and/or outputs.

As used herein, the term “response element” is used to describe the aspect of any element related to the gas reformulation system that is capable of responding to a signal

### Gas Reformulation System

This invention comprises a system for the effective reformulation of gas derived from the gasification of carbonaceous feedstock. The initial gas to be input into this system will generally comprise a complex mixture of hydrocarbon molecules of varying length. The chemical composition and the contaminant quality of the gas will depend on the composition of the feedstock, the process used to generate the gas and the conditions in the gasification system. Some gasifiers are designed for a one step process, wherein various forms of heat are used to generate the gas in a single chamber. Other gasifiers generate the gas in a multi-step process, in either different regions of one chamber or different chambers or some combination thereof. Either system might include some pre-processing of the raw off-gas, generally due to the source of heat in the gasification chamber.

One primary objective of these design strategies is to optimize the effective exposure of the amounts of raw syngas and/or preformulated gas to the reactive species in the gas energizing zone. The greater the degree of effective exposure, the greater the efficiencies of energy transference, and hence, the greater the percent conversion of the preformulated gas into gas of a designed chemical composition in the most overall cost effective manner.

Examples of design strategies include the design of the entire system. For example, important design strategies comprise the flow pattern (turbulence) of the preformulated gas relative to the gas energizing field and particularly the amount of gas that passes through this field in a particular amount of time. One example of these strategies is the system design whereby the preformulated gas passes through plasma generating electric arc(s). Another example is the system design wherein a plasma torch is positioned in a manner that the plasma plume flows counter-current to and directly down into the preformulated gas. In another embodiment, the preformulated gas passes through sequential or parallel gas energizing fields.

The reformulating system of the invention is designed to optimize the amount of preformulated gas that is reformulated into a product gas. In one embodiment, the effectiveness of this process is expressed by the term, Gas Reformulation Ratio, which comprises the amount of reformulated product gas divided by the amount of preformulated or initial reactant gas×100=%. In one embodiment, the Gas Reformulation Ratio is 95% or greater. In one embodiment, the Gas Reformulation Ratio is 90% or greater. In one embodiment, the Gas Reformulation Ratio is 85% or greater. In one embodiment, the Gas Reformulation Ratio is 80% or greater. In one embodiment, the Gas Reformulation Ratio is 75% or greater. In one embodiment, the Gas Reformulation Ratio is 70% or greater. In one embodiment, the Gas Reformulation Ratio is 65% or greater. In one embodiment, the Gas Reformulation Ratio is 60% or greater. In one embodiment, this concept is expressed as a ratio of the value of the reformulated gas as compared to the initial gas. In one embodiment, the value is the energetic value in terms of electricity generation.

In order to effectively reformulate initial gas into gas of a designed composition, this invention comprises one or more “gas reformulating zones,” and one or more “gas stabilizing zones.” A gas stabilizing zone optionally comprises heat transfer means to capture heat from the gas as it cools. The system optionally comprises one or more “gas additive zones,” generally located upstream of a gas reformulating zone, with or without mixing. It also optionally comprises one or more “gas cleaning zones,” generally located downstream of a gas stabilizing zone.

For the purposes of clarity, these zones are described separately. It is understood, however, that these zones are generally contiguous and interrelated within the system, that the system is not limited to comprising discrete, physically separated zones, although this remains an alternative option. Depending upon the design of a particular embodiment, they will be more or less separated. In addition, for ease of reference only, the zones have been named according to the process step that takes place predominantly in that zone. One skilled in the art will appreciate, however, that due to the nature of the reformulation process other process steps may also take place to a lesser extent in that zone.

A system that effectively reformulates gas must be able to raise the energy of the initial gas molecules so that they begin to reformulate. In particular, reaction intermediates are initiated. The energetic processes of a reaction are represented by a curve such as shown below.

As one skilled in the art would appreciate, the arrow points to a representation of energy that is required to induce the gaseous molecules of an initial chemical composition to begin to reformulate into molecules of a designed chemical composition. The dotted line represents the energy required when a catalyst is used to lower the amount of energy required to bring about the reformulation of the molecules. One skilled in the art appreciates that, at a general level, sufficient energy will be required to be imparted to the initial gas molecules to drive them to break their bonds and reformulate into reformulated molecules and atoms. Under the appropriate conditions, if the reformulated molecules and/or atoms are allowed to mix thoroughly, the atoms will recombine according the relative concentrations of the species present. Moreover, if a significant amount of the preformulated gas passes through the energizing field, a significant amount of the gas will be reformulated.

To accomplish the objective of effectively reformulating gas, one skilled in the art can appreciate that the following four chemical processes occur throughout the reformulation of a gas: 1) initiation of the intermediates; 2) propagation of at least a portion of the intermediates; 3) termination of the intermediates; and 4) product gas stabilization.

A gas reformulation process can be envisioned to entail four general processes. In the first process, reactants such as initial gaseous molecules and energy sources (including but not limited to free electrons, and other energized or activated species such as ions and free radicals) are brought together through mixing and reach a state of species-to-species contact. As a result of such contact and a sufficient energy level of the mixture, the interaction of the reactants leads to the formation of chemical intermediates. While some of the intermediates may react together and terminate, at least a portion of the intermediates undergo another step, in which the intermediates react between themselves with or without the participation of the reactants to produce other intermediates, resulting in a chain of chemical reactions. In another process, the intermediates are terminated by chemical and/or physical means and yield specific products. In the fourth and final step, the products formed are stabilized when specific chemical and/or physical conditions are maintained.

The initiation of intermediates may therefore be considered as the dominant process that occurs early within the gas reformulating zone where an intermediate-inducing means (an energy source) is provided and brought into contact with a gas entering the gas reformulating zone. Mixing, energy transfer, and/or radiation which enables the transformation of the reactants into initial intermediates. The reactants can be said to be excited.

The intermediate propagation step may be considered to be another major process that occurs in the gas reformulating zone where the initial intermediates react between themselves to produce other intermediates. It is possible for these intermediates to form a chain of reactions with one group of intermediates being derived from the previous one.

In general, the intermediate termination processes can be considered to occur at the end of the gas reformulating zone and, in some embodiments, may even be considered to define the outer edges of the zone wherein the chemical and/or physical conditions are changed such that the chain reactions are consequently stopped from proceeding further. It would be understood, however, that termination processes may take place in other regions of the gas reformulating zone depending on the specifics of the process, the reactants/intermediates and the stability of the final product. At the end of the chain reactions reached either by controlled termination or by undisturbed progression, specific products are formed.

The gas stabilizing zone may be considered to be located where product stabilization is the dominant process and may be defined as a zone where specific conditions are maintained in order to stabilize the products formed at the termination of the recombining of the intermediates. These products are normally desired for specific applications. If different products are required, effort may be made to adjust the intermediate termination point since different points of the chain reaction course correspond to different intermediates which in term yield different products upon termination and stabilization.

There are many intermediate inducing means. These include thermal heating, plasma plume, hydrogen burners, electron beam, lasers, radiation, etc. In situations where the reactant molecules have sufficient energy to rearrange in the presence of a catalyst and are brought in contact with such a catalyst, the catalyst can be seen to play the role of an intermediate inducing means. The common feature of Energy Sources that provide intermediate inducing means is to cause chemical changes to reactants and proceed along a pathway to final products. The intermediates formed can therefore differ between different intermediate inducing means and have different levels of activation.

There are a number of ways of elevating the energy of the initial gas to a level such that the molecules will reformulate into the molecules of a designed chemical composition. Heat can be added to the initial gas. Activated species, such as the electrons and positive ions found in plasma or produced from a hydrogen source can be used to transfer the energy required to cause the molecules in the initial gas and process additives, “the preformulated gas,” to reformulate into reformulated molecules and atoms.

As noted above, there are various catalysts known to one skilled in the art that can be used to lower the amount of energy that must be required to cause the molecules to reformulate. Catalysts such as dolomite, olivine, zinc oxide and char are examples of some commonly used catalysts.

This invention provides a smart, integrated gas reformulating system for efficient, deliberately planned reformulation of an initial gas with associated characteristic characteristics (e.g. chemical composition) into an output gas with characteristic characteristics designed for a specific downstream purpose. Optimization includes the most overall cost effective manner of accomplishing the reformulation, including upfront costs such as electricity and downstream costs such as processing contaminated catalysts.

### The Gas Reformulating System Process:

(1) senses directly or indirectly the adequacy of the characteristic parameters of the initial gas including but not limited to the chemical composition, humidity, flow rates, etc. Optionally, the system may sense characteristic and/or parameters of upstream and/or downstream systems or input or outputs thereof;
(2) modifies various input parameters to the reformulation process (e.g. optionally increases or decreases appropriate amounts of process additives, modifies the amount of electricity, etc.) based on the sensed characteristic parameters of the initial gas and the desired parameters of the output gas;
(3) generates one or more gas reformulating zones comprising sufficient energetic species that can interact with the off-gas molecules (the initial gas or preformulated gas) to transfer energy to the gaseous molecules such that the majority of the gaseous molecules reformulate into reformulated molecules and atoms;
(4) in the reformulating zone, promotes efficient mixing of the initiated gaseous molecular constituents (the initiated intermediates) such that they recombine into a chemical composition determined by the relative concentrations of the species present in the reformulated gas;
(5) provides a stabilizing zone, whereby the newly formed molecules are de-energized, for example, cooled or removed from the influence of catalysts or gas energizing sources, and thus stabilized to maintain the desired characteristics; and
(6) provides a control system for overall control of the gas reformulation process.

The system and method of gas reformulation may be used to reformulate a substantial amount of off-gas such as produced from gasification of carbonaceous feedstock into a reformulated gas comprising optimal levels of molecules such as carbon monoxide and hydrogen and minimal levels of unwanted molecules.

In the ensuing description, the following parts of the Gas Reformulating System are considered in greater detail. The basic process will be taught beginning with a description of the “gas reformulating zones;” and “gas stabilizing zones.” The strategy and tactics for optimizing the extent and efficiencies of gas reformulation will be described with a discussion of gas manipulators including catalysts and other gas manipulators. Optional features for inclusion in the system include “gas additive zones,” and “gas cleaning zones.” Finally the description will discuss the design of gas reformulating chamber and a control system to manage all of the above processes.

### A Gas Reformulating Zone

The reformulating zone is the zone within the system wherein the preformulated molecules that are sufficiently energized to reformulate into molecular species of a designed chemical composition occurs. In general, this zone is designed such that it incorporates means for causing turbulence and mixing during the reformulating process.

### Gas Energizing Sources

Gas energizing sources provide the initial energy required to overcome the molecular bonding energies of the initial gas and the process additives within the gas reformulating system (the preformulated gas), thus serving to reformulate these molecules into reformulated molecules and eventually the molecules of designed chemical composition, such as CO and H₂. These energizing sources serve to provide energy for initiation of the reactive intermediates, and when required, to provide energy to support propagation of the intermediates.

Various elements are envisioned within this invention for the provision of the gas energizing zones. The energy levels required to meet the requirements of the Gas Reformulation Energy depend on a variety of factors including but not limited to the characteristics (e.g. composition) of the initial gas, the process additives, and the presence of catalysts. Means to increase the temperature, residence time and/or turbulence and mixing are also envisioned for inclusion in designing and creating this zone.

Energy required for gas energizing in order to induce intermediates to become reactive can be provided by various sources referred to as energizing sources, thermal heating, plasma, hydrogen burners, electron beams, lasers, radiation, etc. Their common feature is to cause chemical changes to reactants and proceed along a pathway to final products.

### Sources of Plasma

Plasma provides a source of energy mostly in the form of electrons and positively charged ions that can interact with the preformulated gas to supply Gas Reformulation Energy to the molecules.

In one embodiment of the invention, one or more plasma-based sources (e.g. plasma torches), operated in conjunction with or without other gas energizing sources, are used to raise the energy of the initial gas to a level sufficiently high for gas reformulation, and thus provide a gas energizing zone. The appropriate energy level depends on a variety of factors including but not limited to the characteristics of the initial gas and the process additives, and is readily determined by a worker skilled in the art.

Although heat contributes to the process, a significant portion of the majority of the energy is supplied by the reactive species in the plasma. In one embodiment of the invention, the temperature is between about 800° C. to about 1200° C. The amount of energy required of the source may be lowered by the use of catalysts.

The one or more plasma sources may be chosen from a variety of types including but not limited to non-transferred and transferred arc, alternating current (AC) and direct current (DC), plasma torches, high-frequency induction plasma devices and inductively coupled plasma torches (ICP). In all arc generating systems, the arc is initiated between a cathode and an anode. Selection of an appropriate plasma source is within the skills of a worker in the art.

The transferred arc and non-transferred arc (both AC and DC) torches can employ appropriately selected electrode materials. Materials suitable for electrodes that are known in the art include copper, tungsten alloys, hafnium etc. The electrode lifetime depends on various factors such as the arc-working areas on the electrodes, which in turn depends on the design of the plasma torch and the spatial arrangement of the electrodes. Small arc-working areas generally wear out the electrodes in a shorter time period, unless the electrodes are designed to be cooled by thermionic emission. The electrodes may be spatially adjustable to reduce any variations in the gaps there between, wherein the variations are caused as the electrodes wear down during their lifetimes.

A variety of gases can be used as a carrier gas for plasma torches including but not limited to air, argon, helium, neon, hydrogen, methane, ammonia, carbon monoxide, oxygen, nitrogen, carbon dioxide, C₂H₂ and C₃H₆. The carrier gas may be neutral, reductive or oxidative and is chosen based on the requirements of the gas reformulation process and the ionization potential of the gas. Selection of an appropriate carrier gas and understanding the means of introducing the carrier gas into the plasma torch can impact its efficiency is within the ordinary skills of a worker skilled in the art. In particular, that a poorly designed introduction of the carrier gas can result in a non-uniform plasma plume, with hot and cold zones.

In one embodiment, the gas reformulating system comprises one or more non-transferred, reverse polarity DC plasma torches. In one embodiment, the gas reformulating system comprises one or more water cooled, copper electrode, NTAT DC plasma torches. In one embodiment of the invention, the gas reformulating system comprises one or more AC plasma torches.

AC plasma torches may be either single-phase or multiple phase (e.g. 3-phase), with associated variations in arc stability. A 3-phase AC plasma torch may be powered directly from a conventional utility network or from a generator system. Higher phase AC systems (e.g. 6-phase) may also be used, as well as hybrid AC/DC torches or other hybrid devices using but not limited to hydrogen burners, lasers, electron beam guns, or other sources of ionized gases.

Multiple phase AC plasma torches generally have lower losses in the power supply. In addition, the rapid movement of the arc along the electrodes due to rail-gun effect can result in improved redistribution of the thermal load between the electrodes. This redistribution of the thermal load along with any cooling mechanisms for the electrodes, allows the use of materials for electrodes having a relatively low melting point but high thermal conductivity, such as copper alloys.

The plasma source may comprise a variety of commercially available plasma torches that provide suitably high flame temperatures for sustained periods at the point of application. In general, such plasma torches are available in sizes from about 100 kW to over 6 MW in output power. In one embodiment, the plasma torch is two 300 kW plasma torches each operating at the (partial) capacity required.

### Hydrogen Burners

In one embodiment of the invention, the gas energizing field is at least partially provided by a hydrogen burner wherein oxygen and hydrogen are reacted to form ultra-high temperature steam (>1200° C.). At these high temperatures, the steam may exist in an ionized form which enhances the gas reformulation process. Hydrogen burners may be operated in conjunction with or without other gas energizing sources such as plasma torches. Activated hydrogen species include the benefit of rapid dispersion of the reactive species and extensive steam cracking, both of which lead to a high conversion of the initial gas at a lower temperature than achieved with plasma.

In one embodiment of the invention, hydrogen burners provide a significant portion of the energizing energy, thereby acting as the primary energizing field element.

The hydrogen for the hydrogen burner may be obtained by electrolysis. The oxygen source may be pure oxygen or air. Other sources for hydrogen and oxygen may also be used as would be readily known to a worker skilled in the art. The design of the burner may utilize standard modeling tools e.g. tools based on computational fluid dynamics (CFD). The burner may also be adapted and sized to fit the requirements of the gas reformulating system taking into account various factors including but not limited to the quantity of gases for reformulation, chamber geometry etc.

In one embodiment of the invention, the hydrogen burner comprises a cylindrical nozzle body, with upper and lower covers coupled to its upper and lower ends respectively and defining a predetermined annular space S in the body. A gas supply pipe is connected to a sidewall of the body such that the pipe is inclined downwards therefrom. The upper cover may be integrated with the body into a single structure, and is provided with a heat transfer part having a thickness sufficient for easy dissipation of heat. A plurality of nozzle orifices, which discharge hydrogen to the atmosphere, is formed through the heat transfer part with an exposing depression formed on the upper surface thereof to communicate with each of the nozzle orifices. An airflow chamber is also defined in the body so that air passes through the chamber. A guide protrusion is formed on the inner surface of the space to guide the current of hydrogen gas to a desired direction in the space. Furthermore, the upper end of the annular space S, which communicates with the lower ends of the nozzle orifices, is configured as a dome shape, thus defining a vaulted guide to guide hydrogen gas to the orifices.

Hydrogen burners operate at a lower temperature and usually mix hydrogen with air. They may also use a oxygen-hydrogen mixture which runs at a significantly higher temperature. This higher temperature can give off more radicals and ions; it also will make the gas highly reactive with hydrocarbon vapor and methane.

In one embodiment of the invention, a hydrogen burner serves as a source of high temperature chemical radicals which can accelerate the reformulation of gaseous hydrocarbons into syngas. The hydrogen burner is operated with an oxidizing agent, with air and oxygen being two common choices. A worker skilled in the art will understand the relative proportion of hydrogen and the oxidizing agent required. In addition to generating high-temperature radicals, the hydrogen burner also generates a controllable amount of steam. Typically, hydrogen burners can be powered with efficiencies similar to a plasma torch.

### Electron Beam Guns

Electron beam guns produce electron beams with substantially precise kinetic energies either by emission mechanisms such as thermionic, photocathode and cold emission; by focusing pure electrostatic or with magnetic fields and by a number of electrodes.

Electron beam guns can be used to ionize particles by adding or removing electrons from the atom. A worker skilled in the art will readily know that such electron ionization processes have been used in mass spectrometry to ionize gaseous particles.

The designs of electron beam guns are readily known in the art. For example, a DC, electrostatic thermionic electron gun is formed of several parts including a hot cathode which is heated to create a stream of electrons via thermionic emission; electrodes which generate an electric field to focus the beam, such as a Wehnelt cylinder; and one or more anode electrodes which accelerate and further focus the electrons. For larger voltage differences between the cathode and anode, the electrons undergo higher acceleration. A repulsive ring placed between the anode and the cathode focuses the electrons onto a small spot on the anode. The small spot may be designed to be a hole, in which case the electron beam is collimated before reaching a second anode called a collector.

### Radiation

Ionizing radiation refers to highly-energetic particles or waves that can ionize an atom or molecule. The ionizing ability is a function of the energy of the individual packets (photons for electromagnetic radiation) of the radiation. Examples of ionizing radiation are energetic beta particles, neutrons, and alpha particles.

The ability of electromagnetic radiation to ionize an atom or molecules varies across the electromagnetic spectrum. X-rays and gamma rays will ionize almost any molecule or atom; far ultraviolet light will ionize many atoms and molecules; near ultraviolet and visible light will ionize very few molecules. Appropriate sources of ionizing radiation are known in the art.

### Recycled Energy

The external energy needed to sustain the gas reformulation process may also be reduced by harnessing any heat generated by the process. The amount of heat generated by the gas reformulation process depends on the characteristics of the initial gas and the reformulated gas. In one embodiment, the heat released during the reformulating of carbon or multi-carbon molecules to mainly CO and H₂ is maximized by optimizing the amount and type (e.g. air, O₂) of process additives injected into the gas reformulating system.

The sensible heat present in the gas leaving the reformulating zone may be captured using heat exchangers in the gas stabilization zone, and recycled to enhance the external efficiency of the reformulation process.

Other energizing sources based on thermal energy or lasers may also be used, as would be evident to a worker skilled in the art.

### Gas Manipulators

Gas manipulators represent embodiments of design strategies seeking to optimize the process of gas reformulation. Gas manipulators include They comprise designs of the chamber that optimize the flow pattern of the preformulated gas relative to the gas energizing field and particularly the amount of gas that passes through this field in a particular amount of time. Another example of a gas manipulator is the system design wherein the energy-providing source (such as a plasma torch) is oriented in a manner relative to the incoming reformulating gas that maximizes mixing between the incoming gas and the energetic species in the energy source. Another example is the location and positioning of process additive nozzles that are designed to increase turbulence and mixing. Another might comprise the arrangement of sequential gas reformulating zones versus parallel gas reformulating zones.

The Gas Manipulators comprise structural devices that have been designed and incorporated into the system to increase the efficiency of the gas reformulation process. Examples include, without limitation, structural devices such as baffles and deflectors that direct the preformulated gas more effectively towards and through the gas energizing field. Other examples include structural devices that increase the turbulence throughout the process that increase the mixing of the energizing sources and the reformulating gas.

The Gas manipulators also include aspects of the system that direct the physical orientation of the energizing source to change the dimensions of the energizing field, e.g., plasma plume directing devices, and/or changes to the energy supplied to a plasma-generating source, the flow rate of the working gas, etc. are non-limiting examples of aspects of the system of the invention that can be modified to effect changes in the dimensions of the preformulated gas energizing field.

Catalytic Gas manipulators increase the efficiencies of the energy transference and include catalysts. One example of a gas manipulator is the system design whereby the preformulated gas passes through plasma generating electric arc(s). Inclusion of the gas manipulators is intended to optimize the balance of the amount of energy expended in the process of providing energy to the preformulated gas with the output that is sufficient to enable the system to reformulate syngas into gas of a designed chemical composition.

There are different categories of gas manipulators.

One category of Gas Manipulators, is referred to as Source Energy Exposure Manipulators. The principal design strategy of this aspect of the invention is to optimize the exposure of the amount of preformulated gas necessary to support the reformulation reactions to the initial source of energy.

Another category of Gas Manipulators is referred to as Mixing Manipulators. The principal design strategy of this aspect of the invention is to optimize the mixing of the reactive species to enhance the energy transference throughout the reformulation process.

Another category of Gas Manipulators is referred to as Catalytic Manipulators. The principal design strategy of this aspect of the invention is to optimize the catalytic activities within the system to enhance the overall effectiveness of the reformulation process.

Overall effectiveness refers to the thoroughness of the reformulation process (as expressed by the Gas Reformulation Ratio) in addition to the overall costs of achieving reformulation. For example, the overall effectiveness takes into account the cost of using a catalyst that might become “poisoned” during the process and the cost of replacing it. It will also take into account the cost of the energy sources.

The Gas Reformulating System of this invention is designed to enhance the efficiency of the reformulation process. The various means of accomplishing this are referred to as “gas manipulators” and they enhance the efficiency, effectiveness and thoroughness of the reformulation process. The reformulation process occurs as the preformulated gas is passing through the chamber of the system, so residence time is a critical aspect determining the efficiency of the process and the thoroughness of the transformation. Factors that accelerate the rate and extent of energy transference throughout the preformulated gaseous molecules and the mixing of the reformulated species, optimize the thoroughness of the transformation prior to the gas exiting the system.

The proximity of the gaseous molecules to the source of energy-providing activated species, such as those provided within the plasma, and/or heat, is dependent upon the amount of time the gaseous molecules are exposed to the source. Means provided within the system that enhance the process of energy transfer throughout the preformulated gas molecules which thereby begin to reformulate, maximizes the number of molecules that will be reformulated. In addition, means that increase the amount of mixing of the activated species/reactive intermediates such that they reform into new chemical species, the composition of which is largely dependent upon the relative concentration of the species present in the reformulated gas, also maximize the amounts of designed molecules that will be generated.

Gas manipulators are designed, positioned and operated to enhance the efficiency of the reformulation process. In some embodiments, the gas manipulators are designed to increase the high turbulence within the system. Increasing turbulence influences the gas by provide thorough mixing of the gas molecules to be energized and those that are in the process of reformulating into new molecules, the chemical composition of which will be determined largely by the relative concentrations of the individual chemical species in a gas reformulating zone.

Gas manipulators can be designed to alter the flow dynamics within the gas reformulating system by targeted redirection of the at least one of the gas energizing zone, the initial gases, process additives and constituents thereof, resulting in changes in their relative spatial distribution and dynamic evolution thereof. The gas manipulators may also be designed to ensure that a high turbulence environment is created in targeted locations to aid the energizing and reformulation processes.

By improving the exposure of the gas energizing field (e.g. plasma plumes) with the initial gas and the process additives, improved reaction processes for the energizing and reformulation is achieved at the lowest possible temperature.

A worker skilled in the art will readily understand that the gas manipulators have to be designed and positioned based on the location of the gas energizing sources and inlets for process additives and on the overall design of the chamber.

### Exposure Manipulators

In some embodiments, the gas manipulators are designed and configured to substantially enhance the exposure of the preformulated gas to the reforming zone. As mentioned earlier, these gas manipulators may be separate structural devices attached to the gas reformulating chamber(s) or be integral to the gas reformulating chambers.

### Chamber Designs for Exposure Manipulation

In one embodiment, the gas manipulators comprise of chamber designs that optimize the flow pattern of the preformulated gas relative to the gas energizing field and particularly the amount of gas that passes through this field in a particular amount of time. This can be achieved by appropriate design of the internal walls of the chamber resulting in differences in the gas reformulation channel, i.e., the gas flow path within a chamber. The gas reformulation channel can be a variety of types including but not limited to the following: straight, curved, diverger-converger and the labyrinth.

Various embodiments of gas reformulation channels are shown in FIGS. 25 to 28. A worker skilled in the art will readily understand that several design variations are possible for each of the embodiments of FIGS. 25 to 28, based on the design of additional features of the chambers, such as for example, the ports for air injection. Design considerations for gas reformulation channels include but are not limited to the exposure to the energy source, cross section area, temperature profile, velocity profile, gas residence time, mixing, and pressure drop.

Referring to FIG. 15A and in accordance with one embodiment of the invention, the chamber is straight and comprises a narrow throat wherein the plasma torch is located. The gases passing through the narrow throat is forced to mix with the reactive ionized plasma carrier gas (the gas energizing zone), thus promoting reformulation. The throat is about the size of the visual portion of the plasma plume, associated with temperatures above 2000° C. The carrier gas exists in an ionized phase at such temperatures and is therefore much more active. Design criteria such as the size of the channel (e.g. its cross section area), velocity and temperature profiles etc., are determined by the chemical processes required for enhanced gas reformulation. Any particulate matter present in the reformulated gas may entrain and accumulate in the secondary portion of the chamber due to the higher velocities at the throat.

The chambers may additionally be designed for ease of separation of the particulate matter. Referring to FIG. 15B and in accordance with one embodiment of the invention, the secondary portion of the chamber is located downwards so that the particulate matter may separate at the bottom and be carried away. Alternatively, the secondary portion of the chamber may be designed to have a tangential introduction of the gas from the primary portion of the chamber so that the resulting swirl flow may promote separation of the particulate matter from the gas stream.

The merits of the designs of FIGS. 15A and 15B may also be achieved with a simplified mechanical design by appropriate placement of a structural device internally. Referring to FIG. 15C and in accordance with one embodiment of the invention, the shape of chamber is unchanged throughout its length and the channel is located substantially in the middle of the chamber to force the off-gas through. As the chamber diameter is fixed, the installation of refractory, and the fabrication and installation of the chamber is simplified. The internal structural device may be well insulated and cooled for optimal performance using methods known in the art such as extra cooling piping, fan and controls.

The plasma plume generated by a single plasma torch is of a certain finite length at several milliseconds time period, after which the ionized gas returns to a non-plasma gas state as its temperature drops below about 2000° C. A worker skilled in the art will understand that the time after which the ionized gas returns to a non-plasma gas state depends on various parameters of the plasma torches including but not limited to the enthalpy of the torch, the gas flows, the temperature of the surrounding gas and the amperage. In gas reformulation chambers with curved type channels, two or more plasma torches may be appropriately located to provide a continuous stream of reactive ionized gas for interaction with the incoming off-gas, resulting in enhanced efficacy of the tar cracking processes.

A variety of designs are possible for curved channels not limited to the embodiments of FIGS. 16A to 16C. In accordance with one embodiment of the invention, the secondary portion of the chamber allows for tangential introduction of gas from the primary portion of the chamber so that the resulting swirl flow promotes the separation of the particulate matter from the gas stream. A worker skilled in the art will readily understand that a multitude of curved channel designs are possible, for example, based on differences in the angles of the curves.

Referring to FIG. 17 and in accordance with one embodiment of the invention, the channel is a divergent-convergent type where the shape of the channel allows for variations in the local conditions such as velocities, pressure, etc. if necessary.

Referring to FIG. 18 and in accordance with one embodiment of the invention, the channel is a labyrinth type. A worker skilled in the art will readily understand that this channel design can accommodate for longer residence time, if necessary.

In one embodiment of the invention, the chamber is a straight, substantially horizontal cylindrical structure operatively linked to a source of gas (e.g. gasifier) through a vertically oriented connector. The walls of the chamber and/or connector may be designed to act as a gas manipulator i.e., to precisely redirect the preformulated gas stream and enhance its interaction with the gas energizing field and optionally the process additives.

In one embodiment of the invention, the chamber is constricted at appropriate locations to enhance the interaction of the preformulated gases with the gas energizing field (e.g. plasma plumes) and/or the process additives. Referring to FIG. 20A and in accordance with one embodiment of the invention, the constriction 3999 within the chamber 3202 is placed slightly above the two plasma torches 3208. Referring to FIG. 20B and in accordance with one embodiment of the invention, the constriction 3999 is more gradual and is positioned such that the plasma torches 3208 fall within the constricted area of the chamber 3202. A worker skilled in the art will readily understand the impact of the different positions of the constriction vis-à-vis the plasma torches.

In one embodiment of the invention, an injector plasma torch with its own injector stream as carrier gas is used to generate an ionized field in a chamber comprising electrodes driven by multiple-phase AC currents, and filled with the preformulated gas to be reformulated. As the preformulated gas passes directly through the chamber, the energizing and reformulation processes are enhanced. Various embodiments of the gas manipulators as described below may still be utilized to ensure that the plumes of the injector plasma torch are directed precisely into the gaps of the primary electrodes.

Referring to FIGS. 21A and 21B, various embodiments of the gas reformulating system may be conceived based on the configurations of the energizing sources, additive streams and gas inputs and gas outputs.

The gas reformulation system may also be designed for separation of the gas stream into smaller streams which undergo parallel reformulation. Referring to FIGS. 24A and 24B, each of the smaller gas streams pass through dedicated reformulation zone created by independent energizing sources. FIG. 24B shows the use of transferred arc torches. FIG. 24C shows that the dedicated reformulation zone for each separate gas stream may be created by multiple gas energizing sources. FIG. 24D shows the embodiment of FIGS. 24A and 24B where mixing elements are introduced in the path of each of the smaller gas streams.

FIGS. 25A-C show three gas reformulating systems where the gas energizing sources are positioned at angles in the reformulation chamber. The sources may either direct its energizing field towards the flow of gas or against it; or combination thereof.

The chamber may further include one or more ports for secondary torch heat sources to assist in the pre-heating or torch heating of the chamber.

### Preformulated Gas Directing Devices

Gas manipulators may enhance the exposure of the preformulated gas with the gas energization field by manipulating directly or indirectly, using active or passive means or both, the spatial distribution of the preformulated gas within the chamber(s) and its dynamic evolution thereof. Such gas manipulators may be separate structural devices. Examples include, without limitation, structural devices such as baffles and deflectors that direct the preformulated gas more effectively towards and through the gas energizing field. Other examples include the design of the chamber to create certain desired fluid dynamic flow paths.

In one embodiment of the invention, gas manipulators are also located at or near the initial gas inlet to ensure that the initial gas is of more uniform composition and/or temperature, and properly mixed with the process additives.

Referring to FIGS. 26A-C, and in accordance with one embodiment of the invention, the gas manipulators comprises of flow restrictors 3999 that alter the flow of the gases entering the chamber 3202. A worker skilled in the art will readily understand that the differences to the gas flow patterns is dependant on various factors including but not limited to the size and shape of the flow restrictors 3999 and their position.

The flow restrictors may be attached to the chamber using various fastening means. In one embodiment of the invention, the flow restrictor is suspended from the top (downstream end) of the chamber. In one embodiment of the invention, the flow restrictor is attached using brackets to the walls of the chamber.

Referring to FIGS. 27A and 27B, and in accordance with one embodiment of the invention, the flow restrictors 3999 extend for substantially the whole length of the chamber 3202 resulting in the formation of an annular space where gas reformulation occurs. As shown in FIG. 42, the flow restrictor 3999 may be rotated using a motor 7001, an example of the use of active means for the direct manipulation of the off-gas streams. The rotation of the flow restrictors may be dynamically controlled, optionally in conjunction with a control system that is designed to regulate and optimize the overall gas reformulation process.

FIGS. 28A and 28B show the three dimensional views of a chamber comprising flow restrictors and directly coupled to a laterally oriented gasifier. The flow restrictors have to be designed to withstand the high temperatures typically present in a chamber.

FIGS. 29A-G show different flow restrictors, in accordance with various embodiments of the invention. In these figures, the plasma torches are shown to be at the same elevation. Alternately, the flow restrictors may be placed above or below the plasma torches. Additive ports are also shown below the torches for the injection of process additives, such as air and steam.

In one embodiment of the invention, as shown in FIG. 29A, the flow restrictor has two helical flights that are designed to induce more cyclical flow mixing of the incoming off-gas and the plasma plume. FIG. 29B shows a flow restrictor with two helical flights but with a different shape, in accordance with one embodiment of the invention. In one embodiment of the invention, as shown in FIG. 29D, one helical flight of the flow restrictor is larger than the other and further induces the cyclical flow and mixing of the off-gas with the plasma plumes. In one embodiment of the invention, as shown in FIG. 29G, the flight spiral only covers half of the restrictor before starting as two new flights.

In one embodiment of the invention, as shown in FIGS. 29C-F, the flow restrictor is attached to a cooling pipe where a cooling medium (e.g. air, water, thermal oil) controls the temperature of the flow restrictor. In one embodiment of the invention as shown in FIG. 29E, additives (e.g. air, steam etc) flow from the top of the support rod to the bottom of the flow restrictor before it enters the off-gas stream. This design allows for cooling of the flow restrictor while pre-heating of the additives prior to their injection.

Referring to FIG. 30A and in accordance with one embodiment of the invention, the chamber comprises gas manipulators in the form of one or more rotational shafts attached to a motor, each shaft comprising one or more disks, which may be carefully weighted for stable rotation. For embodiments with multiple disks on a shaft, the disks may be arranged in an off-set pattern. A worker skilled in the art will readily understand that the disks may incorporate cooling. Flow restrictors such as described above may be attached to the end of the rotational shaft.

FIG. 30B show different types of disks that may be attached to the rotational shaft. Referring to FIG. 30BA, the disk has a section that allows gas to flow from one side of the disk to the other. Referring to FIG. 30BB, the disk has a spiral section that is designed to pull the gases up and into the middle of the chamber. Alternately, the spiral section may be designed to push gases up and out to the edge of the chamber. Referring to FIGS. 30BC and 30BD, the rotational disk is a spoke with multiple blades. A worker skilled in the art will readily understand that the orientation and weight distribution of the blades should be balanced for stable rotation.

FIGS. 31A-C show different embodiments of the rotational shaft such as shown in FIG. 42, wherein the top disk is allowed to rotate on ball bearings and is held in place by supports. Optionally, cooling fluids or additives can be piped through the center of the shaft. In one embodiment of the invention as shown in FIG. 31A, there is a motor on top of one or more supports with a drive shaft attached to a wheel (sprocket) that turns. The mechanical energy turns the disk, and thus the shaft protruding into the chamber.

Referring to FIG. 31B, electromagnets are used either between the supports or as part of the supports to cause rotation. Referring to FIG. 31C and in accordance with one embodiment of the invention, electromagnets are used to stabilize the shaft in the chamber. The electromagnets can be used either as a primary or a secondary means for creating a rotational moment in the shaft and the disks. In one embodiment of the invention, the disk rotates independently of the shaft; for example, the shaft may be stationary or rotating at another speed or even another direction. In one embodiment of the invention, the disk has permanent magnetics and cooling is done on the disk plane as it would be mostly hollow with a thermal fluid cooled ball bearing connection to the shaft.

### Energizing Source Directing Devices

Energizing Source Directing Devices are gas manipulators that direct the physical orientation of the energizing source to change the dimensions of the gas energizing field, e.g., plasma plume directing devices, and/or changes to the energy supplied to a plasma-generating source, the flow rate of the working gas, etc. are non-limiting examples of aspects of the system of the invention that can be modified to effect changes in the dimensions of the gas energizing field.

Gas manipulators may also enhance the exposure of the preformulated gas with the gas energization field by manipulating directly or indirectly, using active or passive means or both, the spatial distribution of the gas energizing field (e.g. plasma plumes) within the chamber(s) and its dynamic evolution thereof. In one embodiment of the invention, this may be achieved by positioning and orientation of the energizing source (e.g. plasma torch).

In one embodiment of the invention, as shown in FIG. 33A, the gas manipulator is a deflector 3998 that redirects the plasma plumes 3997 from a plasma torch 3208. The proper redirection of the plasma plumes is dependent on various design factors of the deflector 3998 including but not limited to its distance from the plasma torch 3208, its angle of orientation vis-à-vis the direction of the plasma plume, its size in comparison to the width of the plasma plume, and its material of construction. Heat resistant materials ensure that the deflector can tolerate the high temperatures present proximal to the plasma torch 3208. A worker skilled in the art will readily know the different materials that can be used to withstand the high plasma temperatures.

Referring to FIG. 33B and in accordance with one embodiment of the invention, the gas manipulator is a Coanda-effect based deflector 3996 used to manipulate the plasma plume 3997.

Referring to FIGS. 34A and 34B, and in accordance with one embodiment of the invention, one or more fluidic jets 3208 (e.g. air nozzles) are used to redirect the plasma plumes 3997 generated by the plasma torch(es) 3208. The fluidic jets are an example of active means used for the direct manipulation of the plasma plumes. In one embodiment of the invention, the fluidic jets are dynamically controlled, optionally in conjunction with a control system that is designed to regulate and optimize the overall gas reformulation process.

FIGS. 35A-D show other embodiments of deflectors that can be used for redirecting the plasma plumes within the chamber. In one embodiment of the invention, as shown in FIGS. 35A-B, the deflector is attached to the plasma torch casing. By adjusting the shape of the deflector, the spread of the plasma plume dispersion may be controlled. For example, the deflector of FIG. 35B gives wider plume dispersion than the deflector of FIG. 35A.

FIGS. 35C-D show embodiments of the invention, where the deflector is not attached to the plasma torch casing. In one embodiment of the invention, as shown in FIG. 35D, the deflector is attached to the rotating shaft. A worker skilled in the art will understand that the finish (e.g. smooth, rough, or angled) of the deflector surface will affect the plume dispersion.

FIGS. 36A-D show different embodiments of the invention where the rotating shaft object has an uneven surface. The number of edges, torches and torch angles can be used to optimize the plasma plume and/or to evenly spread the plasma plume, thus maximizing the plume's contact with the off-gases. In one embodiment of the invention, the plasma torches point directly to the center of the chamber.

In one embodiment of the invention as shown in FIG. 36A, the plasma torches are angled so that at least part of the plasma plumes hits the center object. Alternatively, the plasma plumes may be directed away from the central object. In one embodiment of the invention as shown in FIG. 36B, the shaft object is rotated in the opposite angle of the torch resulting in forcing the plasma plumes toward the outside of the chamber.

In one embodiment of the invention as shown in FIGS. 36C-D, the plasma plume is bounced off deflectors towards the central shaft. The deflectors may be mounted on the plasma torch casing, as shown in FIG. 36C, or on the walls of the chamber, as shown in FIG. 36D. The shafts in FIGS. 36C-D may be rotated in either direction.

Optionally, ports for mounting plasma torches may be fitted with a sliding mounting mechanism to facilitate the insertion and removal of the plasma torch(es) from the chamber and may include an automatic gate valve for sealing the port following retraction of the plasma torch(es). In one embodiment of the invention, the ports for the tangentially mounted plasma torches are located above the air inlets to provide maximum exposure to plasma torch heat. Such mounting mechanisms may be modified to allow for adjustability of the position of the gas energizing sources.

Referring to FIG. 38A and in accordance with one embodiment of the invention, a plasma torch 3208 is positioned such that the gases injected into the chamber 3202 flows counter-current to the plasma plumes generated thereby. A worker skilled in the art should readily understand the variations in the spatial distribution of the plasma plumes when the orientations and positions of the plasma torches are varied.

In one embodiment of the invention, the gas energizing sources (e.g. plasma torches) are placed so that the resulting zone (e.g. plasma plumes) is directed perpendicular to the direction of the flow of the initial gases. In one embodiment of the invention, the chamber is substantially cylindrical and the plasma plumes are directed radially, perpendicular to the substantially axial flow of the initial gas stream. Alternately, the initial gas stream may be directed radially while the plasma plumes are directed axially along the substantially cylindrical gas refinement chamber. In one embodiment of the invention, the chamber is substantially cylindrical and the plasma plumes are directed tangentially, perpendicular to the substantially axial flow of the initial gas stream.

FIG. 39 shows cross-sectional views of cylindrical gas reformulating chambers with various arrangements of the gas energizing sources resulting in associated changes in the shapes and dimensions of the resulting gas energizing fields. In one embodiment of the invention, the gas energizing sources used may be either AC or DC plasma torches. FIG. 39A shows two gas energizing sources directed tangentially into the chamber. Referring to FIG. 39B, the chamber comprises three electrodes with an arc passing between them. The gas passes through this arc and plasma is formed and the gas is reformulated. FIG. 39C shows a similar embodiment as FIG. 39B, except that there is a central grounded electrode where the arc from the electrodes on the wall arc to. A worker skilled in the art will understand that the ground electrode is electrically shielded except at contact point. FIG. 39D shows one exemplary embodiment where the chamber comprises a plurality of gas energizing sources (either point directly in the middle as shown, or in a swirl pattern) sufficient to ensure that substantially all of the gas passing through the chamber is energized. FIGS. 39E and 39F are similar to the embodiments of FIGS. 39B and 39C respectively, but with six torches (3 or 6 phase). Higher number of torches can also be similarly considered for the embodiments of FIGS. 39B, 39C, 39E and 39F.

FIG. 40 shows two exemplary embodiments of the invention, wherein the initial gas and/or preformulated gas stream is introduced into a reformulation chamber directly in through the gas energizing field created by a gas energizing source.

Gas manipulators at least partially manipulate the spatial distributions of the preformulated gas and the gas energizing field relative to one another, and their dynamic evolutions.

### Mixing Manipulators

In some embodiments, the gas manipulators are designed and configured to substantially enhance the mixing of the reformulating gas and the energetic species in the gas energizing field. Additionally, the gas manipulators may also enhance the turbulence throughout the process resulting in improved mixing.

In one embodiment of the invention, the location and positioning of process additive nozzles that are designed to increase turbulence and mixing.

In one embodiment, the gas manipulators are one or more baffles located in the chamber to induce turbulence and thus mixing of the reformulating gas. Different baffle arrangements are known in the art and include but are not limited to cross bar baffles, bridge wall baffles, choke ring baffle arrangements and the like. Baffles may also be located at or near the initial gas inlet to ensure that the initial gas is of more uniform composition and/or temperature, and properly mixed with the process additives.

Referring to FIGS. 43A-B, turbulence may be created either prior to or after the gas energizing sources. FIG. 43C shows three exemplary embodiments of means for creating turbulence: (i) passive grid; (ii) an active grid utilizing a rotating shaft; and (iii) a shear generator. FIGS. 45 and 46 show additional exemplary embodiments of means for generating turbulence.

In one embodiment, the gas manipulators comprise the design of the positioning of the energizing sources, which can contribute to the mixing of the reformulating gas and the energetic species in the gas energizing field. The energizing sources may thus be positioned to optimize the gas reformulation process; the positioning depends on various factors including but not limited to the design of the gas reformulating chambers (chamber). In one embodiment of the invention, two plasma torches are positioned tangentially to create the same swirl directions as air and/or oxygen inputs do. In one embodiment of the invention, two plasma torches are positioned at diametric locations along the circumference of the chamber.

The arrangement of the process additive (the chemical composition contribution of which is discussed below) inputs is based on a variety of factors including but not limited to the design of the chamber, the desired flow, jet velocity, penetration and mixing. Various arrangements of the process additive ports and ports for the gas energizing sources are contemplated by the invention.

For example, the oxygen inputs or ports, steam inputs or ports and ports for the gas energizing sources may be arranged in layers around the circumference of the chamber, allowing for the tangential and layered injection of gas energizing zones, oxygen and steam. In one embodiment, there is provided nine oxygen source(s) ports arranged in three layers around the circumference of the chamber. In one embodiment there is provided two steam input ports arranged in two layers around the circumference of the chamber and diametrically positioned. In embodiments where the air and/or oxygen input ports are arranged in layers, they may be arranged to maximize the mixing effects.

In one embodiment of the invention, the air and/or oxygen input ports are positioned tangentially, thus allowing the lower level input ports to premix the gas, torch heat it up, and start a swirl motion in the gas. The upper level air input ports can accelerate the swirl motion thereby allowing a re-circulating vortex pattern to be developed and persisted.

Referring to FIG. 44 and in accordance with one embodiment of the invention, the gas to be reformulated enters tangentially into the reformulation chamber resulting in formation of swirls. The embodiment also shows an exemplary gas manipulator shaped and positioned to enhance the exposure of the gas stream with the gas energizing source.

In one embodiment, the lowest level of air input ports is composed of four jets which will premix the gases generated from a lower gasifier and torch heat it up. The other upper two levels of air nozzles provide main momentum and oxygen to mix gases and torch heat up to the temperature required. The arrangements of steam inputs or ports is flexible in number, levels, orientations and angle as long as they are located in a position to provide optimized capabilities to temperature control.

The oxygen and/or steam input ports may also be positioned such that they inject oxygen and steam into the chamber at an angle to the interior wall of the chamber which promotes turbulence or a swirling of the gases. The angle is chosen to achieve enough jet penetration based on chamber diameter and designed air input port flow and velocity. The angle may vary between about 50° and 70°.

The air input ports maybe arranged so that they are in the same plane, or arranged in sequential planes. In one embodiment the air input ports are arranged in lower and upper levels. In one embodiment, there are four air input ports at the lower level and another six air input ports at upper level in which three input ports are slightly higher than the other three to create cross-jet mixing effects.

Optionally, air can be blown into the chamber angularly so that the air creates a rotation or cyclonic movement of the gases passing through the chamber. The gas energizing sources (e.g. plasma torches) may be angled to provide further rotation of the stream.

In one embodiment of the invention, the air and/or oxygen and/or steam inputs comprise high temperature resistance atomizing nozzles or jets. Appropriate air nozzles are known in the art and can include commercially available types such as the type A nozzles and type B nozzles illustrated in FIGS. 47-48. The nozzles may be of a single type or different types. The type of nozzles may be chosen based on functional requirements, for example a type A nozzle is for changing the direction of air flows for creating the desired swirls and a type B nozzle is for creating high velocity of air flow to achieve certain penetrations, and maximum mixing.

The nozzles can be designed to direct the air at a desired angle. In one embodiment, the air jets are positioned tangentially. In one embodiment, angular blowing is achieved by having a deflector at the tip of the input nozzle, thus allowing the inlet pipes and flanges to be square with the chamber.

In one embodiment of the invention, one or more air jets (e.g. air swirl jets) are positioned at or near the initial gas inlet to inject a small amount of air into the initial gas and create a swirling motion in the initial gas stream by taking advantage of the injected air's velocity. The number of air swirl jets can be designed to provide substantially maximum swirl based on the designed air flow and exit velocity, so that the jet can penetrate to the center of the chamber.

### Catalytic Manipulators

Catalytic manipulators include catalysts and increase the efficiencies of the energy transference. A catalyst increases the rate of a chemical reaction, by lessening the time needed to reach equilibrium. A catalyst works by providing an alternate and easier pathway from reactants to products by a variety of mechanisms, but in each case by lowering the activation energy of the reaction. Homogeneous catalysts are present in the same phase as the reactants and function by combining with the reacting molecules or ions to form unstable intermediates. These intermediates combine with other reactants to give the desired product and to regenerate the catalyst. Heterogeneous catalysts are present in a phase different from that of the reactants and products. They are usually solids in the presence of gaseous or liquid reactants. Reactions occur at the surface of heterogeneous catalysts. For this reason catalysts are usually finely divided solids or have particle shapes that provide a high surface-to-volume ratio. The cracking of petroleum and the reforming of hydrocarbons are common industry applications of the use of heterogeneous catalysts. One difficulty in the use of heterogeneous catalysts is that most of them are readily “poisoned” wherein impurities in the reactants coat the catalyst with un-reactive material or modify its surface, so that the catalytic activity is lost. Frequently, but not always, the poisoned catalyst can be purified and used again.

The use of appropriate catalysts in the gas reformulating system may reduce the energy levels required for the gas reformulation process, by providing alternate reaction pathways. The precise pathway offered by a catalyst will depend on the catalyst used. The feasibility of the use of catalysts in gas reformulation systems, in general, depends on their lifetimes. Lifetimes of catalysts may be shortened by ‘poisoning’, i.e., the degradation in their catalytic capabilities due to impurities in the gas.

The gas reformulating system may be designed to allow for easy replacement of the catalysts. In one embodiment of the invention, catalysts are incorporated into the gas reformulating chambers in the form of a bed mounted on a sliding mechanism. The sliding mechanism allows for easy removal and replacement of the catalyst bed. The bed may be inserted at various locations in the gas reformulating system.

In one embodiment of the invention, off-gas from a gasification chamber which is at a high temperature contacts a catalyst which effectively lowers the energy threshold required for gas reformulation, such that the off-gas stream undergoes reformulation prior to exposure to a gas energizing field. In one embodiment of the invention, therefore, the gas reformulation system comprises a catalyst at a location upstream of the gas energizing source(s). In one embodiment, as disclosed in FIG. 57 catalytic beds are inserted before and/or after the gas energizing sources (e.g. plasma torches).

The catalytic capability will also depend on the temperature of operation. The appropriate operating temperature ranges for various catalysts are known in the art. The gas reformulating system may incorporate adequate cooling mechanisms to ensure that the catalysts are maintained within their optimal operating temperature ranges. Additives such as steam, water, air, oxygen or recirculated reformulated gas may be added to help increase or decrease the temperature near the catalyst beds. A worker skilled in the art will understand that the specific additive chosen to control the temperature will depend on the position of the catalyst bed and the gas temperatures thereat.

The irregularity of the catalyst surface and good contact between the large organic molecules and the surface will increase the opportunity for reformulation into smaller molecules, such as H₂ and CO.

Catalysts that may be used include but are not limited to olivine, calcined olivine, dolomite, nickel oxide, zinc oxide and char. The presence of oxides of iron and magnesium in olivine gives it the ability to reformulate longer hydrocarbon molecules. A worker skilled in the art will understand to choose catalysts that do not degrade quickly in the gas environment of the system.

Both nonmetallic and metallic catalysts may be used for enhancing the reformulation process. Dolomites in calcined form are the most widely used nonmetallic catalysts for reformulation of gases from biomass gasification processes. They are relatively inexpensive and are considered disposable. Catalytic efficiency is high when dolomites are operated with steam. Also, the optimal temperature range is between about 800° C. and about 900° C. The catalytic activity and the physical properties of dolomite degrade at higher temperatures.

Dolomite is a calcium magnesium ore with the general chemical formula CaMg(CO₃)₂ that contains ˜20% MgO, ˜30% CaO, and ˜45% CO₂ on a weight basis, with other minor mineral impurities. Calcination of dolomite involves decomposition of the carbonate mineral, eliminating CO₂ to form MgO—CaO. Complete dolomite calcination occurs at fairly high temperatures and is usually performed at 800° C.-900° C. The calcination temperature of dolomite, therefore, restricts the effective use of this catalyst to these relatively high temperatures.

Olivine, another naturally occurring mineral has also demonstrated catalytic activity similar to that of calcined dolomite. Olivine is typically more robust than calcined dolomite.

Other catalytic materials that may be used include but are not limited to carbonate rocks, dolomitic limestone and silicon carbide (SiC).

Char can act as a catalyst at lower temperatures. In one embodiment of the invention, the gas reformulation system is operatively linked to a gasifier, and at least part of the char created within the gasifier is moved to the gas reformulating system for use as a catalyst. For embodiments utilizing char as catalyst, the catalyst bed is typically placed before the energizing zone such as provided by plasma torches.

FIG. 49 shows a fixed bed of char used as a catalyst in the reformulation chamber. The char used for catalysis may be obtained from a gasifier as shown in FIG. 50. This may be particularly applicable when the gas reformulating chamber is operatively linked to a gasifier and used to reformulate the gases generated therefrom. The char may be moved to a residue conditioning chamber or a carbon converter once it loses its catalytic properties.

FIG. 51 shows one exemplary configuration of a gasifier operatively linked to a plasma torch-based gas reformulating chamber wherein the char created in the gasifier aids in catalytic cracking of the off-gases created by gasification. The catalytic cracking achieved in the latter stage of the gasifier is followed by further gas reformulation due to the exposure of the gas with the gas energizing field created by the plasma torch. Various types of gasifiers as would be readily known to worker skilled in the art such as fluidized bed gasifiers and entrained flow gasifiers may also be utilized.

In one embodiment of the invention, the initial gas is heated to a temperature of 900-950° C. and passed over a nickel-based catalyst whereby tar components and light hydrocarbons including methane are converted into CO and H₂. Nickel-based catalysts may be particularly useful when the initial gas contains minimal amounts of sulphur species (such as hydrogen sulphide), such as for example, gas produced by gasification of biomass. Life-times of nickel-based catalysts may be enhanced by the use of promoters such as rare metals.

In one embodiment of the invention as shown in FIG. 52, a catalytic bed is installed right after the gasifier and transforms the majority of the volatiles. The inlet temperature of the catalytic bed may be raised from 600 to 950° C. by combusting a small fraction of the volatiles. The outlet temperature of the catalytic bed is expected to drop to 850° C. and the outlet gas is fed into the gas energizing field for further reformulation. The gas energizing zone may be operated at 1000° C. for this purpose and the resulting syngas is sent to the recuperator to start the subsequent gas cleanup process.

In one embodiment of the invention as shown in FIG. 53, the volatiles from the gasifier passes through the gas energizing zone wherein the temperature is between about 900° C. and about 1000° C. The catalytic bed is used for further reformulation. The temperature of the syngas is expected to drop to 850° C. at the exit of the catalytic bed. It is then sent to a heat exchanger or recuperator which forms a part of the gas stabilization zone.

In one embodiment of the invention as shown in FIG. 54, heat recovery is achieved before the catalytic bed. The majority of the volatiles from the gasifier are reformulated in the gas energizing zone at temperatures of about 1000° C. The hot output gas passes through a heat exchanger (or a recuperator) to preheat process air whereupon its temperature drops to around 700° C. The cooled syngas is then heated to 900° C. by combusting a small fraction of it and fed into the catalytic bed. The resulting syngas at 850° C. is sent optionally for further gas cleanup.

For embodiments where the catalytic bed is placed prior to energizing field, the gas temperature is typically appropriate for high catalytic activity. However for embodiments where the catalytic bed is after the energizing field, such as produced by plasma torches, the gas temperature might be too high for most typical catalysts such as olivine, dolomite, and many others. The gas temperatures may be reduced to appropriate levels (to avoid the degradation of the catalyst beds) by the circulation of cooling fluids, as shown in FIG. 55. Appropriate cooling fluids may include but are not limited to recirculated reformulated gas (as shown in the embodiment of FIG. 56), water and steam.

For embodiments where the catalyst bed is after the recuperator (heat exchanger) the recirculated stream of reformulated gas may be inserted either prior or after the recuperator.

In one embodiment of the invention, the reforming zone comprises a catalyst bed and the catalytic manipulators are also designed to enhance the exposure of the preformulated and/or reformulating gas to the catalyst bed.

### A Gas Stabilizing Zone

This system provides one or more stabilizing zones whereby the newly formed molecules are de-energized (e.g. cooled or removed from the influence of catalysts or energizing sources) to ensure they maintain the desired characteristics e.g. the designed chemical composition.

The temperature of the gas entering the stabilizing zone will range from about 400° C. to over 1000° C. The temperature may optionally be reduced by a heat exchange system in the stabilizing zone of the gas reformulating system, which recovers heat from, and thus cools, the reformulated gas. Such a reduction in the gas temperature may be necessitated by downstream applications and components.

Referring to FIG. 22B, the gas reformulating chamber 3002 the stabilization zone may be specifically shaped to facilitate the de-energization and stabilization of the newly formed molecules. The gas reformulating chamber 3002 is a generally cylindrically shaped chamber having a bulbous expansion downstream of the plasma or optionally proximal to the one or more reformulated gas outlets 3006. The bulbous expansion by allowing for the de-energization of the gas and thereby stabilized the newly formed molecules.

### Optional Heat Recycling Means

Heat may be recovered in the stabilization zone or downstream from the stabilizing zone. The recovered heat may be used for various purposes, including but not limited to the following: heating the process additives (e.g. air, steam) for the gas reformulation process; generating electricity in combined cycle systems. The recovered electricity can be used to drive the gas reformulation process, thereby alleviating the expense of local electricity consumption. The amount of heat captured depends on a variety of factors including but not limited to the characteristics (e.g. chemical composition, flow rates) of the initial gas and reformulated gas.

In one embodiment of the invention, the heat recovered from the stabilizing zone of the gas reformulating system is supplied to a gasification system operated in conjunction with the gas reformulating system. The heat exchanger may be operated in conjunction with a control system optionally configured to minimize energy consumption and maximize energy production/recovery, for enhanced efficiency.

In one embodiment of the invention, a gas-to-fluid heat exchanger is used in the stabilizing zone to transfer the heat from the reformulated gas to a fluid resulting in a heated fluid and a cooled gas. The heat exchanger comprises means (e.g. conduit systems) for transfer of the reformulated gas and fluid to and from the heat exchanger. Suitable fluids include but are not limited to air, water, oil, or another gas such as nitrogen or carbon dioxide.

The conduit systems may optionally employ one or more regulators (e.g. blowers) appropriately located to manage the flow rates of the reformulated gas and the fluid. These conduit systems may be designed to minimize heat losses to enhance the amount of sensible heat that is recoverable from the reformulated gas. Heat loss may be minimized, for example, through the use of insulating barriers around the conduits, comprising insulating materials as are known in the art and/or by reducing the surface area of the conduits.

In one embodiment of the invention, the gas-to-fluid heat exchanger is a gas-to-air heat exchanger, wherein the heat is transferred from the reformulated gas to air to produce a heated exchange air. In one embodiment of the invention, the gas-to-fluid heat exchanger is a heat recovery steam generator, wherein the heat is transferred to water to produce heated water or steam.

Different classes of heat exchangers may be used including shell and tube heat exchangers, both of straight, single-pass design and of U-tube, multiple pass design, as well as plate-type heat exchangers. The selection of appropriate heat exchangers is within the knowledge of a worker of ordinary skill in the art.

As particulate matter may be present in the gas, the gas-to-air heat exchanger is typically designed for a high level of particulate loading. The particle size may vary typically from about 0.5 to about 100 microns. In one embodiment depicted in FIG. 58, the heat exchanger is a single pass vertical flow heat exchanger 5104B, wherein the reformulated gas 5020 flows in the tube side and the air 5010 flows on the shell side. The reformulated gas 5020 flows vertically in a “once through” design, which minimizes areas where build up or erosion from particulate matter could occur. The reformulated gas velocities should be maintained to be high enough for self-cleaning, while still minimizing erosion, and may vary from about 3000 to about 5000 mm/sec.

Due to the significant difference in the air input temperature and hot product gas, each tube in the gas-to-air heat exchanger preferably has individual expansion bellows to avoid tube rupture. Tube rupture may occur where a single tube becomes plugged and therefore no longer expands/contracts with the rest of the tube bundle. In those embodiments where the air pressure is greater than the reformulated gas pressure, tube rupture presents a high hazard due to problems resulting from air entering gas mixture.

After heat is recovered in the gas-to-fluid heat exchanger, the cooled reformulated gas may still contain too much heat for the systems further downstream. Selection of an appropriate system for further cooling of the product gas prior to conditioning is within the knowledge of a worker skilled in the art.

In one embodiment, as depicted in FIG. 59, the hot reformulated gas 5020 passes through the gas-to-air heat exchanger 5103 to produce a partially cooled reformulated gas 5023 and heated exchange-air 5015. The air input to the heat exchanger may be supplied by a process air blower. The partially cooled reformulated gas 5023 undergoes a dry quench step 6103, where the addition of a controlled amount of atomized water 6030 results in further cooled product gas 5025.

The cooling of the reformulated gas may also be achieved using a wet, dry or hybrid cooling system. The wet and dry cooling systems may be direct or indirect. Appropriate cooling systems are known in the art and as such a worker skilled in the art in view of the requirements of the system would be able to select an appropriate system.

In one embodiment, the cooling system is a wet cooling system. The wet cooling system can be direct or indirect. In cooling systems that utilize indirect wet cooling, a circulating cooling water system is provided which absorbs the heat from the reformulated gas. The heat is expelled to the atmosphere by evaporation through one or more cooling towers. Alternatively, to facilitate water conservation, the water vapor is condensed and returned to the system in closed loop.

In one embodiment, the cooling system is a dry cooling system. The dry cooling system can be direct or indirect. In one embodiment, the dry cooling system is a draft dry cooling system. Although, dry cooling will add modestly to the cost of the facility, it may be preferred in areas with a limited water supply.

In one embodiment, the syngas cooler is a radiant gas cooler. Various radiant gas coolers are known in the art and include those disclosed in U.S. Patent Application No. 20070119577, and U.S. Pat. No. 5,233,943.

The reformulated gas may also be cooled down by direct water evaporation in an evaporated such as quencher.

The exit temperature of the reformulated gas may also be reduced by re-circulating, through appropriately located inlets, cooled reformulated gas to the stabilizing zone of the gas reformulating system for mixing with newly produced reformulated gas.

### Optional Gas Additive Zones

The chamber may optionally comprise one or more process additive ports for injection of process additives, such as oxygen sources, carbon dioxide, other hydrocarbons or additional gases, into the chamber. Oxygen sources known in the art include but are not limited to oxygen, oxygen-enriched air, air, oxidizing medium, steam and other oxygen sources as would be readily understood by a worker skilled in the art. In one embodiment, the chamber comprises one or more port(s) for air and/or oxygen inputs and optionally one or more ports for steam inputs.

The optional addition of process additives such as air, steam and other gases, may also be achieved without inlets dedicated to their injection. In one embodiment of the invention, the process additives may be added into the source of gas or conduits wherefrom the Gas Reformulating System obtains its initial gas stream. Process additives may also be added to the chamber through the gas energizing sources, such as plasma torches.

Optionally, ports or inlets may be provided so that reformulated gas not meeting quality standards may be re-circulated into the chamber for further processing. Such ports or inlets may be located at various angles and/or locations to promote turbulent mixing of the materials within the chamber.

One or more ports can be included to allow measurements of process temperatures, pressures, gas composition and other conditions of interest.

Optionally, plugs, covers, valves and/or gates are provided to seal one or more of the ports or inlets in the chamber 3002. Appropriate plugs, covers, valves and/or gates are known in the art and can include those that are manually operated or automatic. The ports may further include appropriate seals such as sealing glands.

### Optional Gas Cleaning Zones

The system optionally comprises one or more gas cleaning zones, located downstream of the gas stabilizing zone. Embodiments of the invention comprising one or more gas cleaning zones incorporate means of injecting substances into the chamber that clean the gas, prior to its exit from the system. For example, oxygen and/or steam can be atomized by high temperature resistance atomizing nozzles and injected into the chamber to clean the stabilized, reformulated gas.

### Optional Further Processing

The stabilized reformulated gas stream may undergo further processing before being utilized in a downstream application, stored or flared off. For example, the reformulated gas may be passed through a gas conditioning system where particulate matter, acid gases (HCl, H₂S) and/or heavy metals may be removed, and the temperature and/or humidity of the gas may be adjusted. For example, dust particles, if present, may be removed from the gas using a venture scrubber, including an electro-filter or fabric baghouse filter.

The reformulated gas may also be passed through a homogenization chamber, the residence time and shape of which is designed to encourage mixing of the reformulated gas to attenuate fluctuations in the characteristics thereof.

### Gas Reformulating Chambers

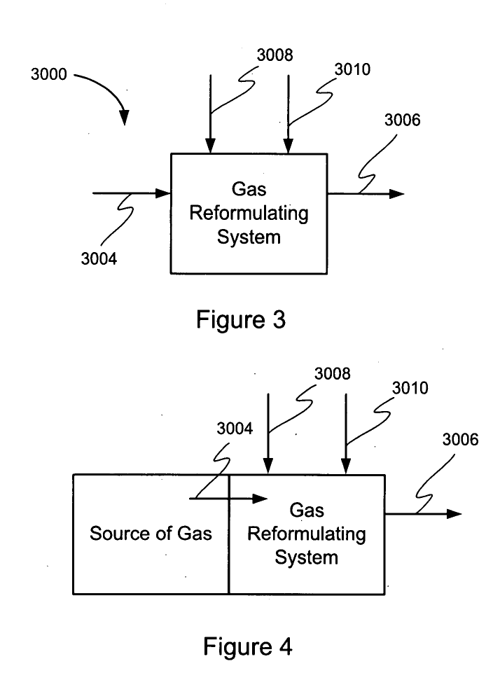

Referring to FIG. 3 and in accordance with one embodiment of the invention, the chamber 3002 of the Gas Reformulating System 3000 comprises one or more initial gas inlets 3004, one or more reformulated gas outlet(s) 3006, one or more gas energizing sources (e.g. plasma sources) 3008, and optionally one or more process additive (e.g. oxygen) inputs 3010, gas manipulators (not shown in the figure), and a control system.

In one embodiment as shown in FIG. 4, the Gas Reformulating System 3000 is designed so that the chamber 3002 is coupled directly to a source of gas (e.g. gasifier, gas storage tank) and in gaseous communication therewith. To facilitate maintenance or repair, the Gas Reformulating System 3000 may optionally be reversibly coupled to the gasifier such that the Gas Reformulating System 3000, if necessary, may be removed.

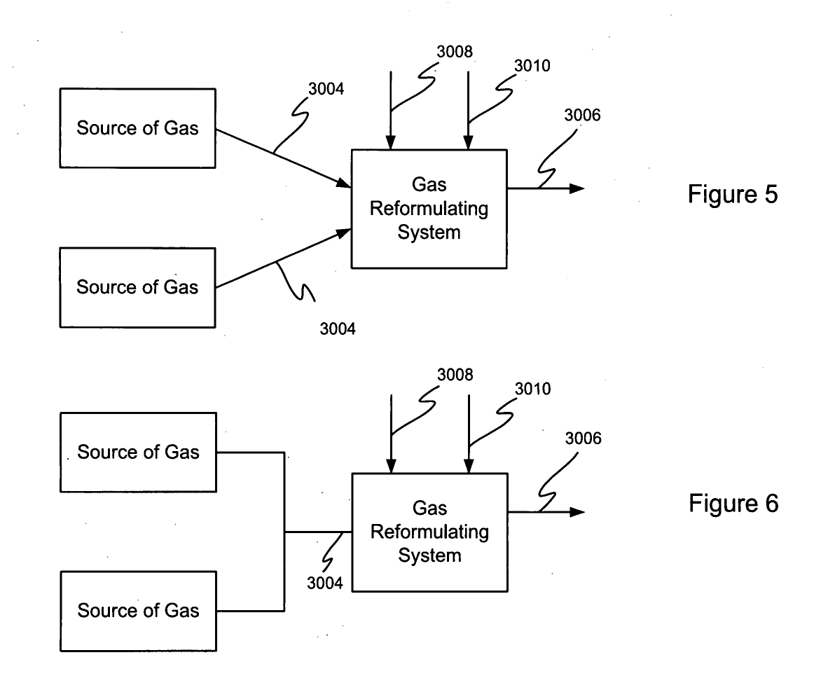

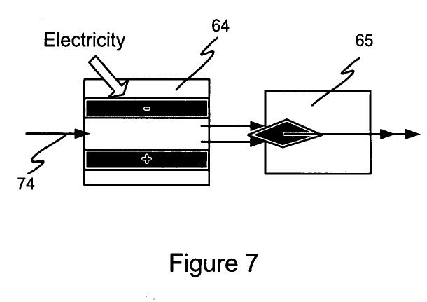

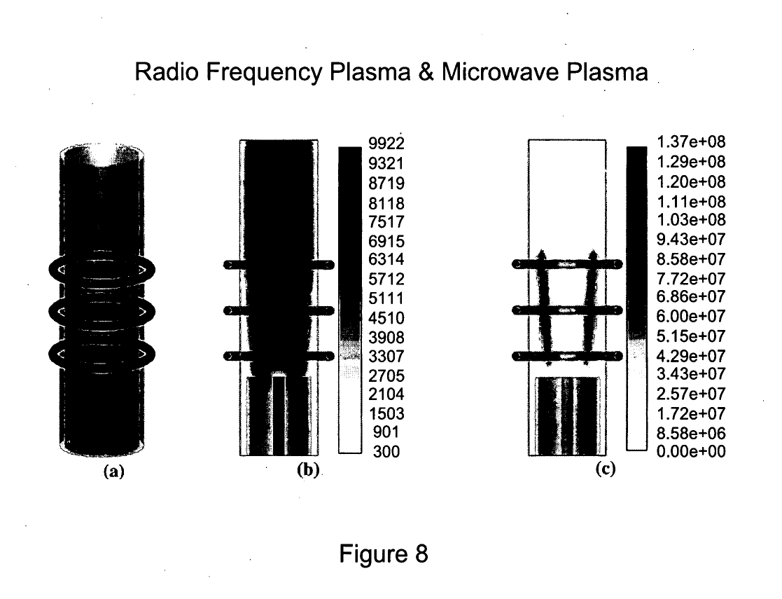

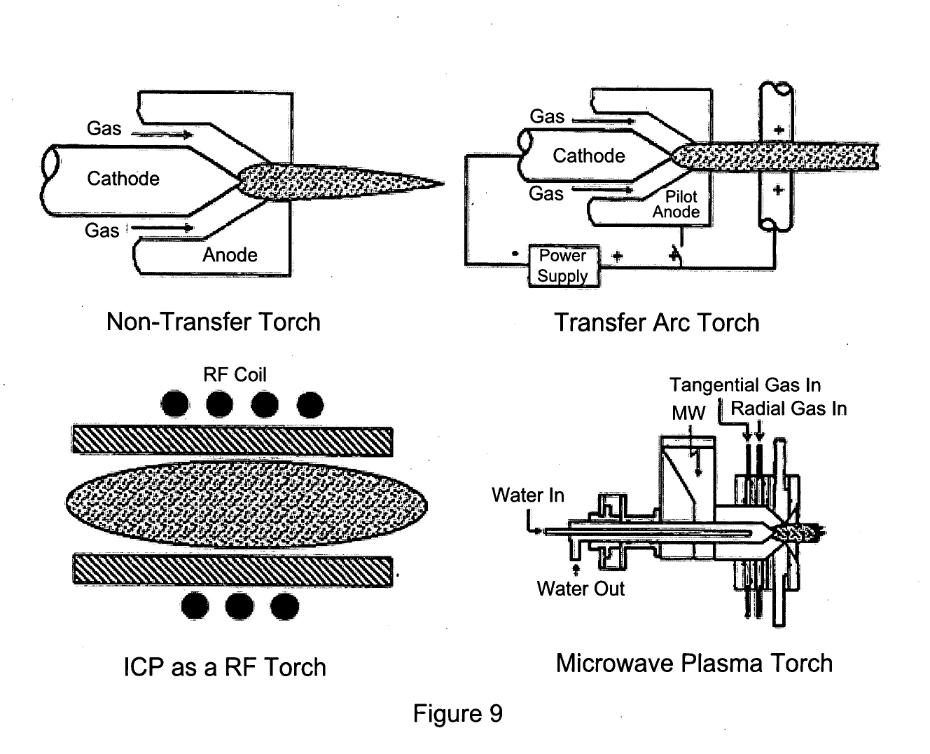

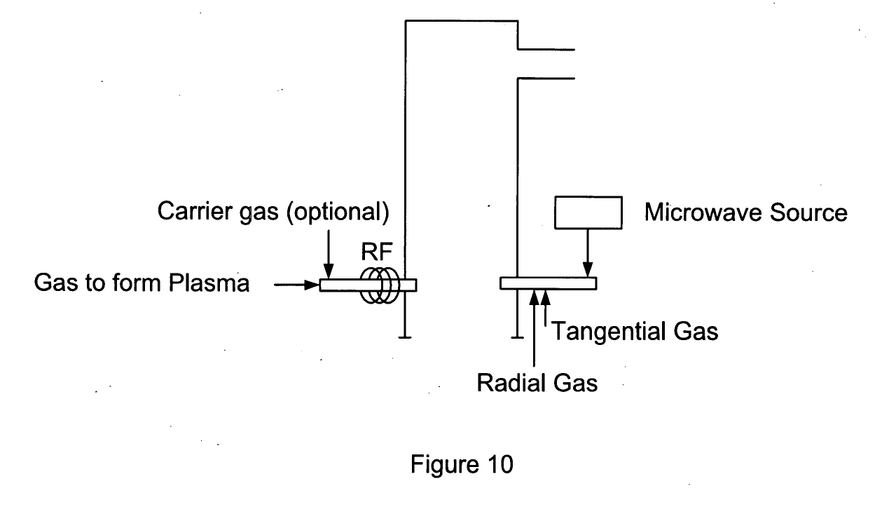

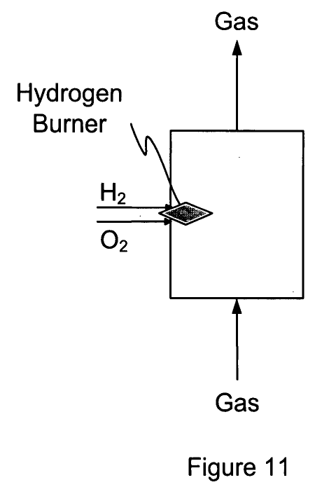

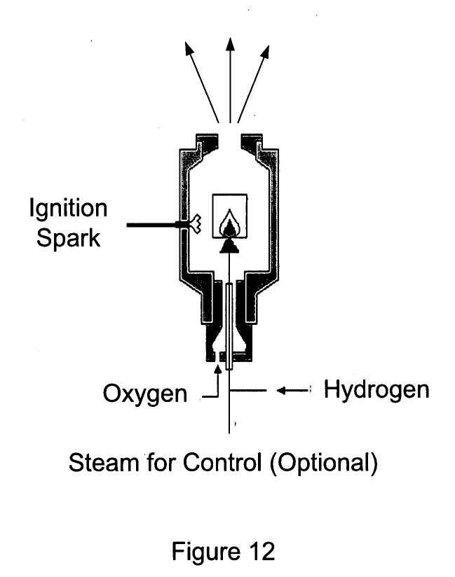

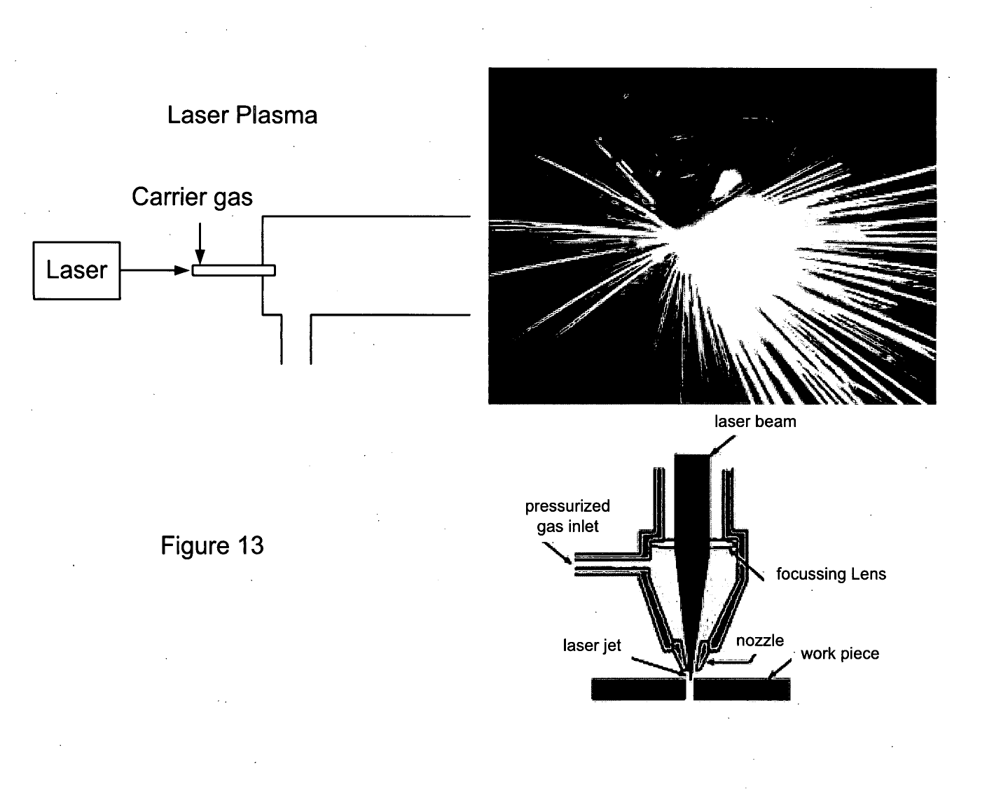

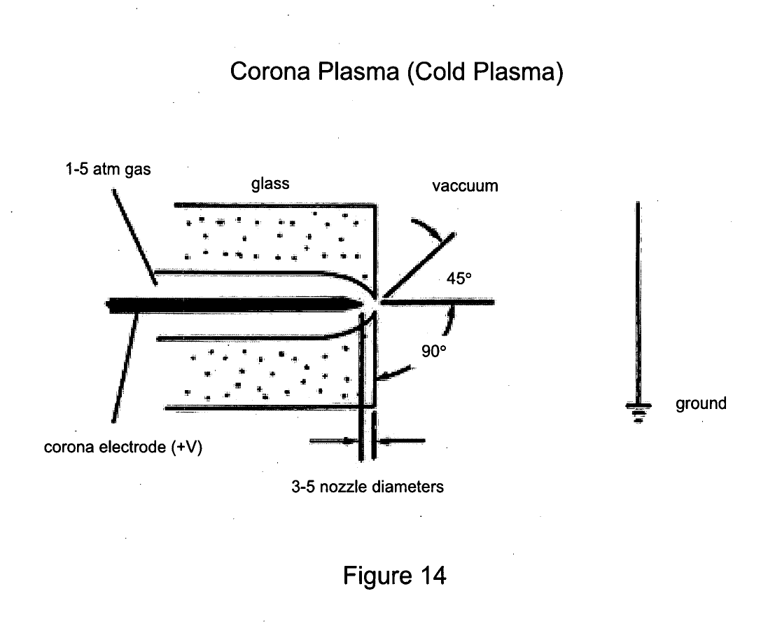

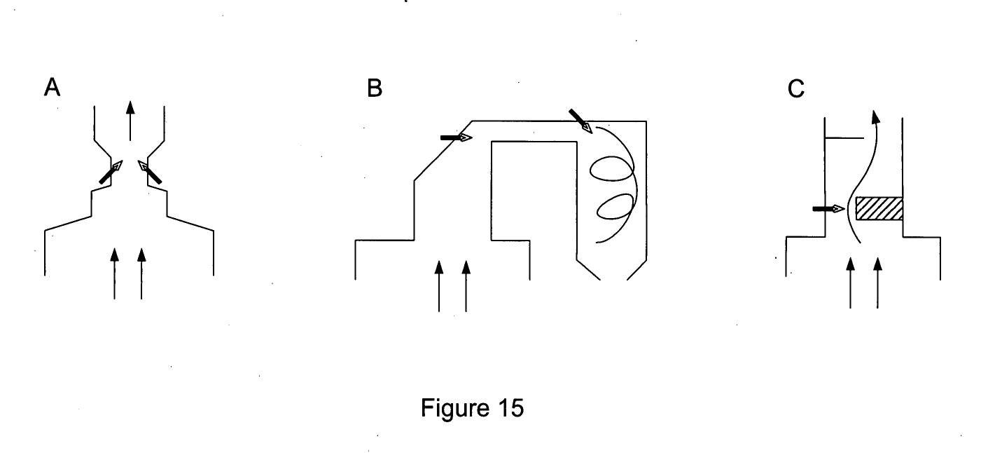

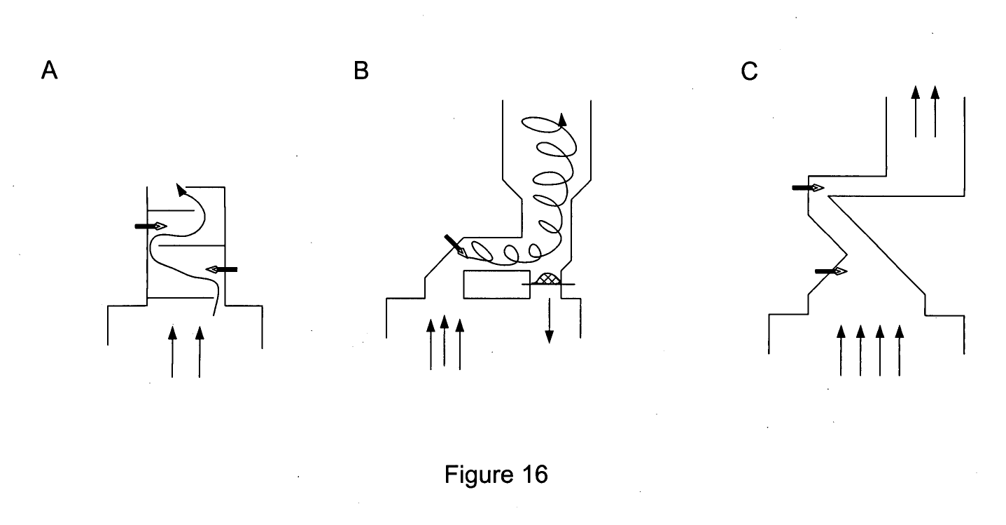

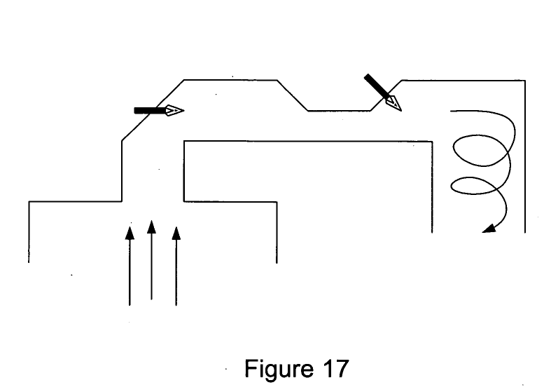

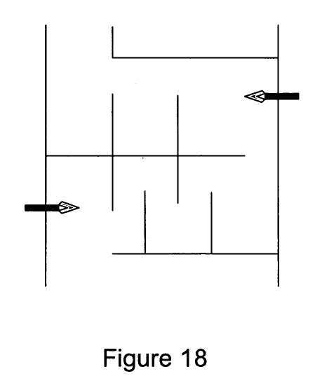

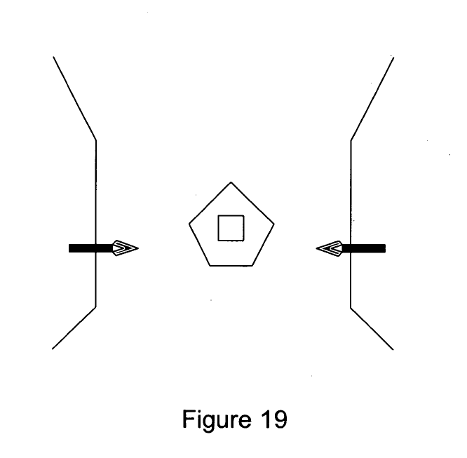

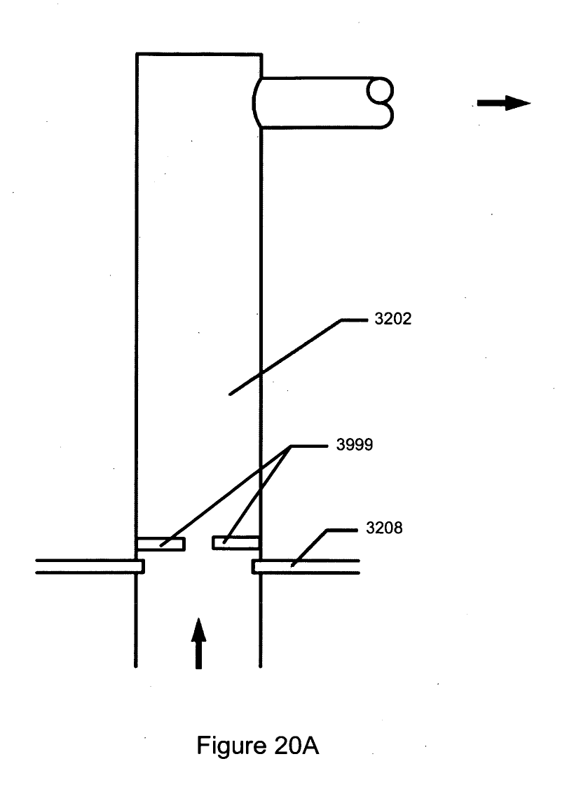

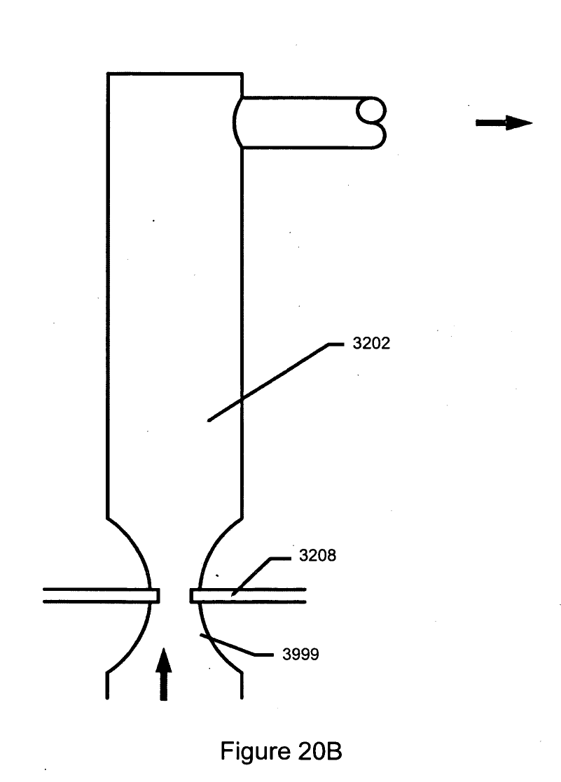

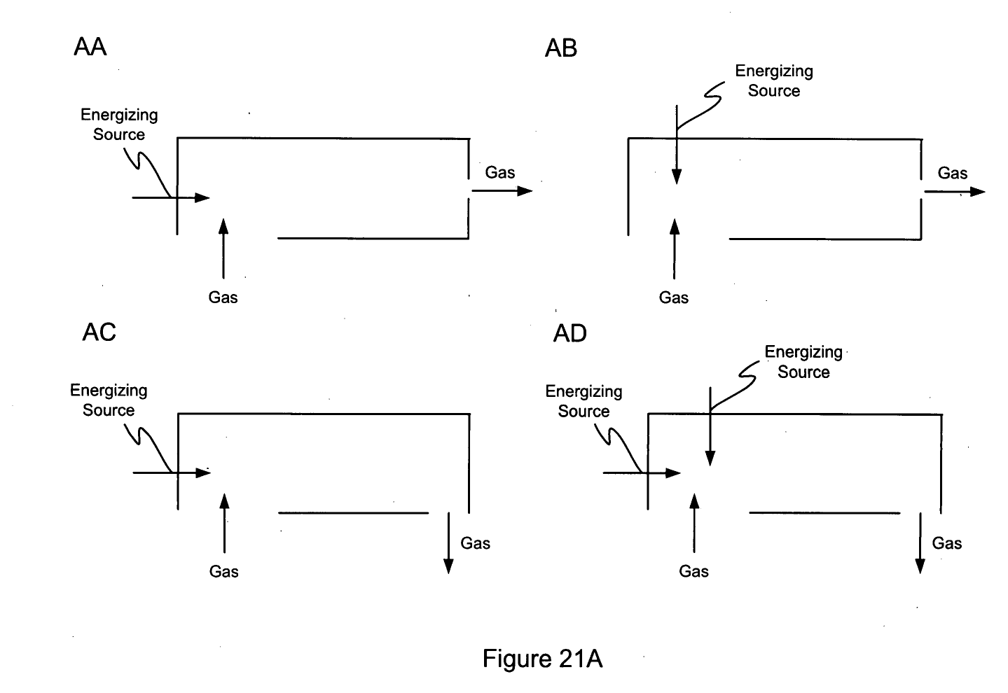

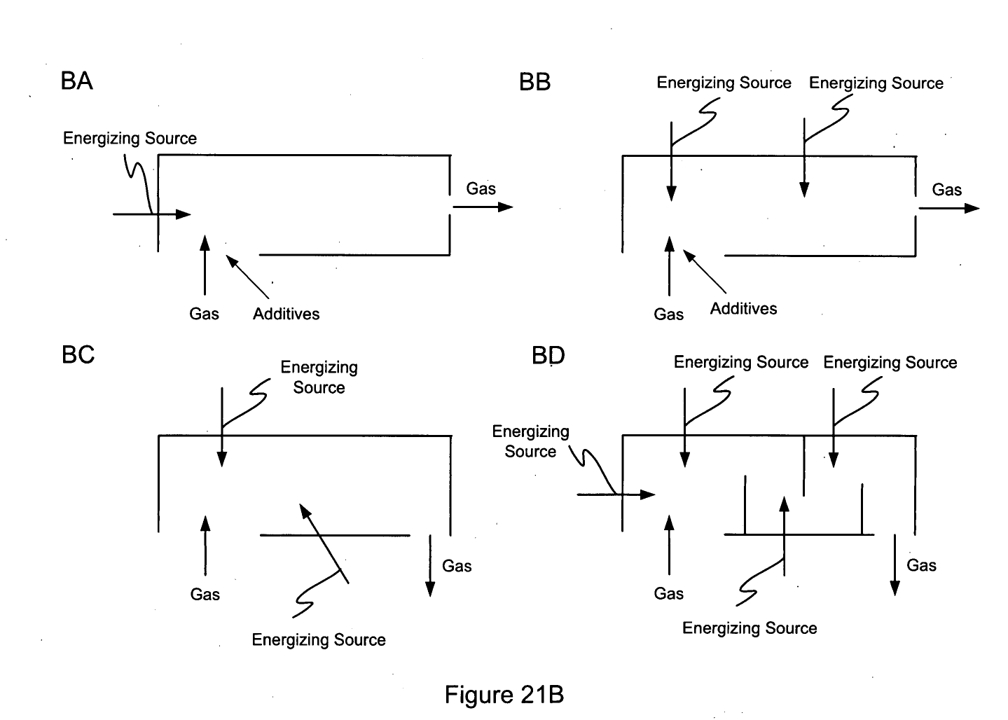

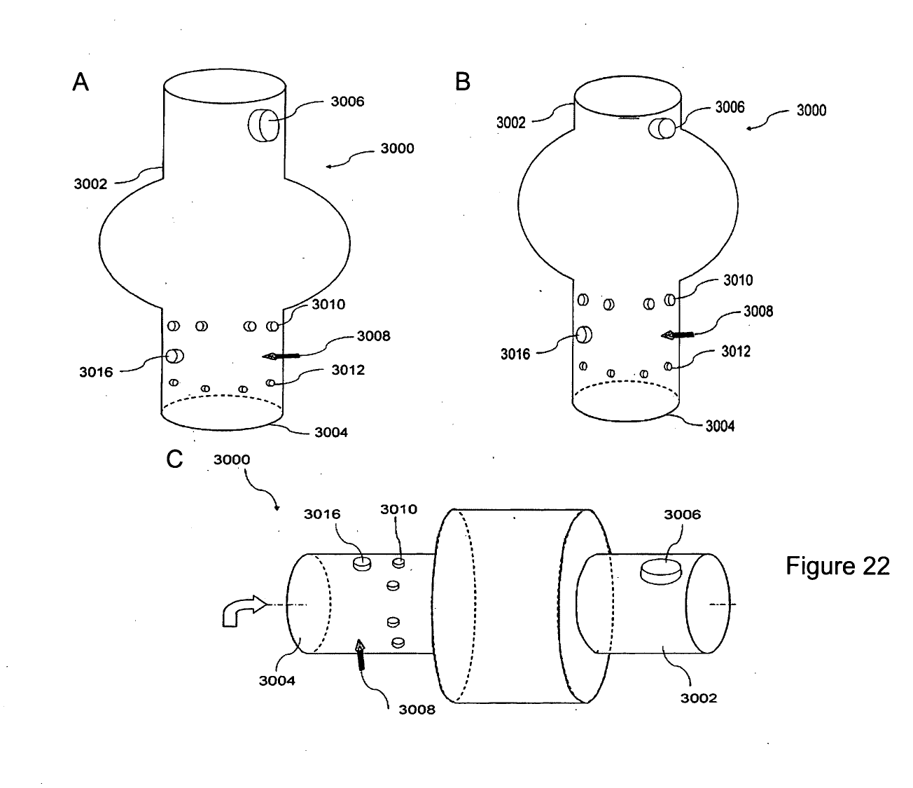

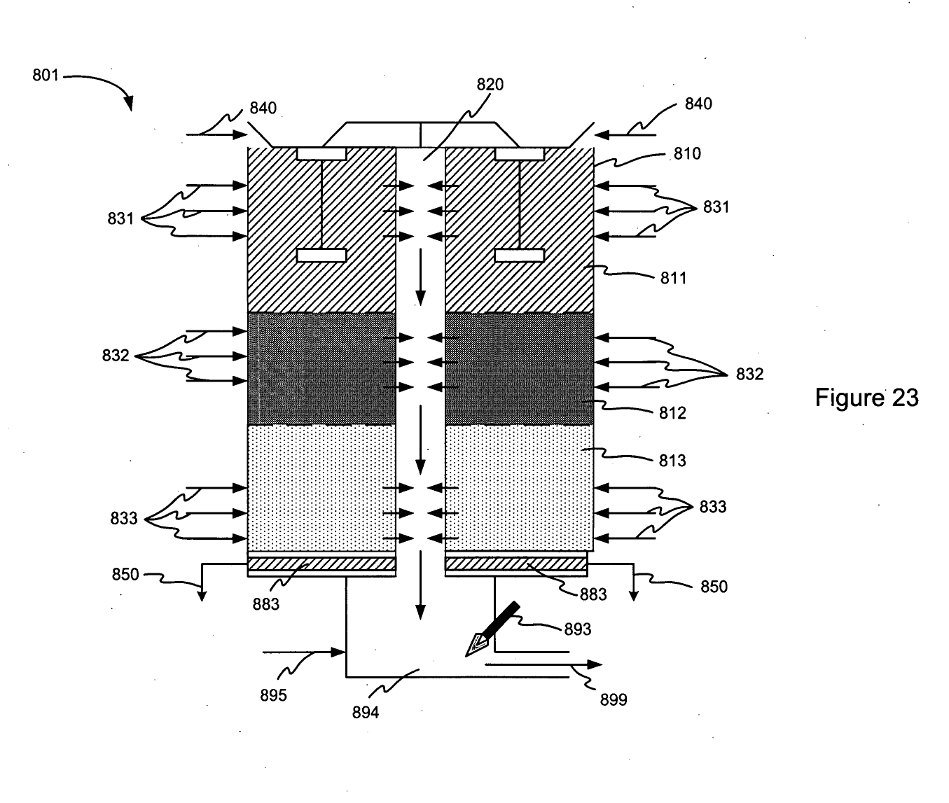

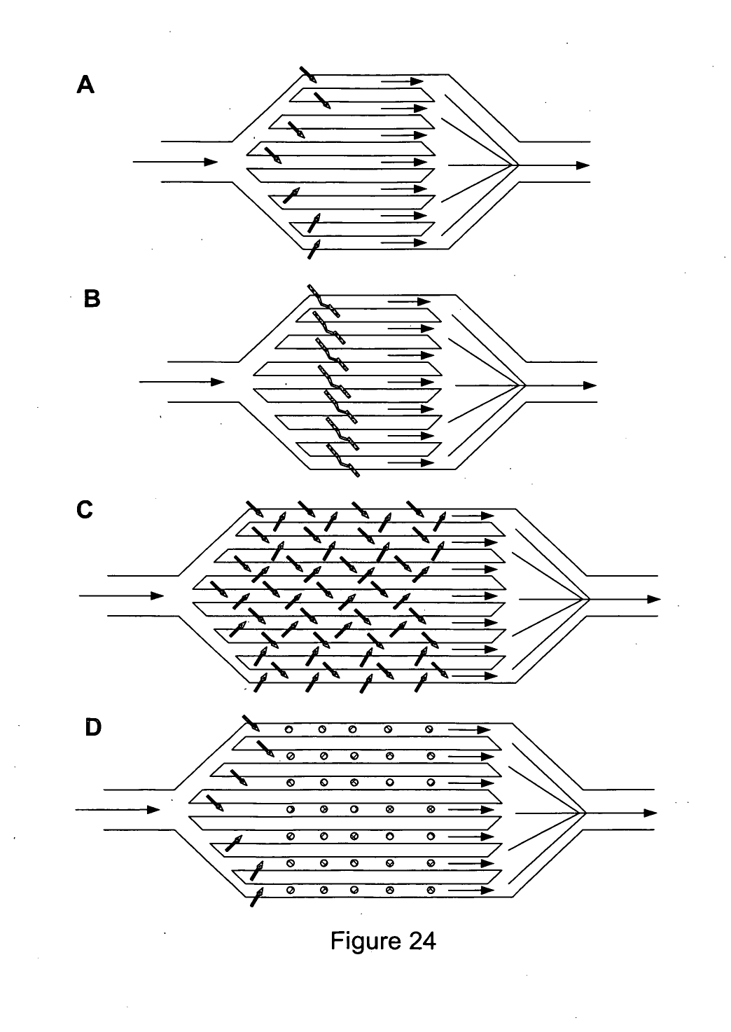

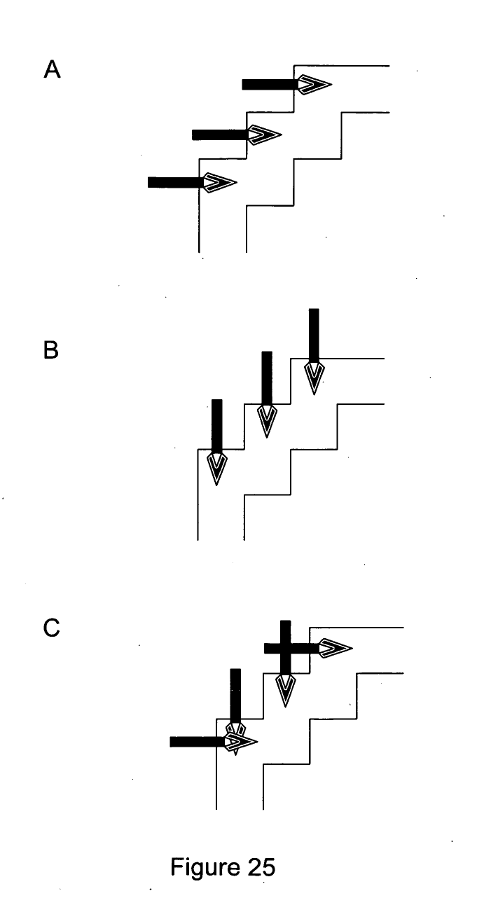

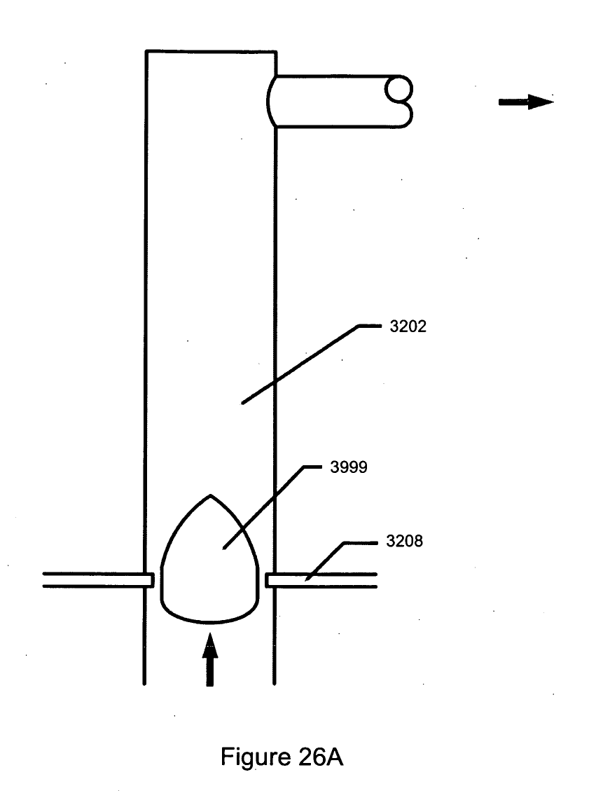

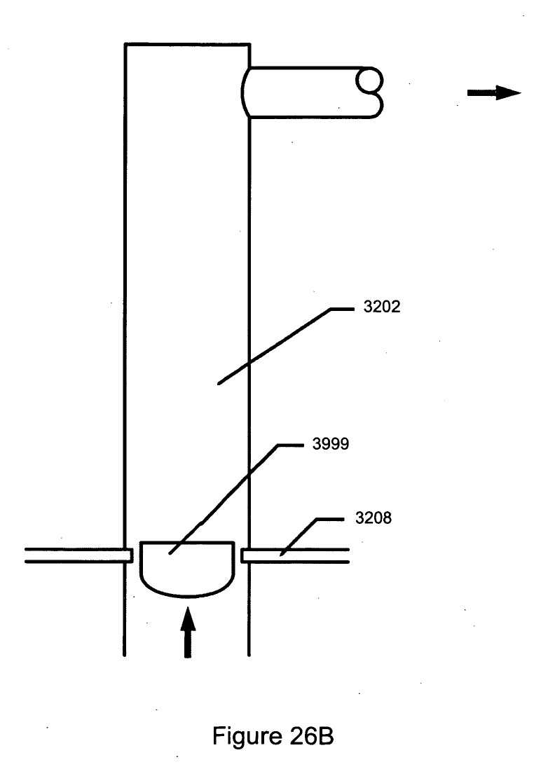

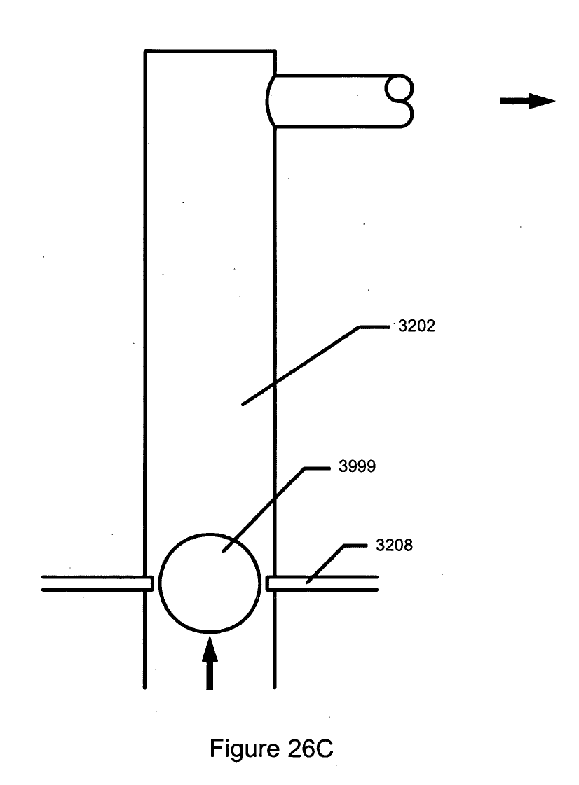

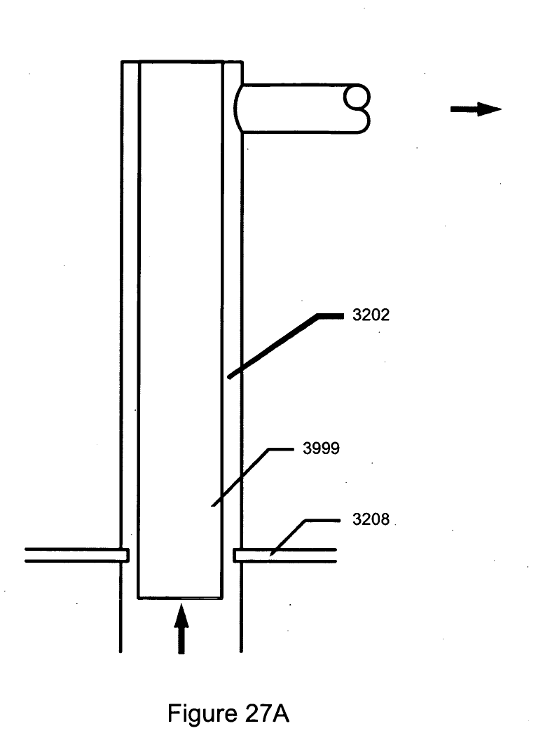

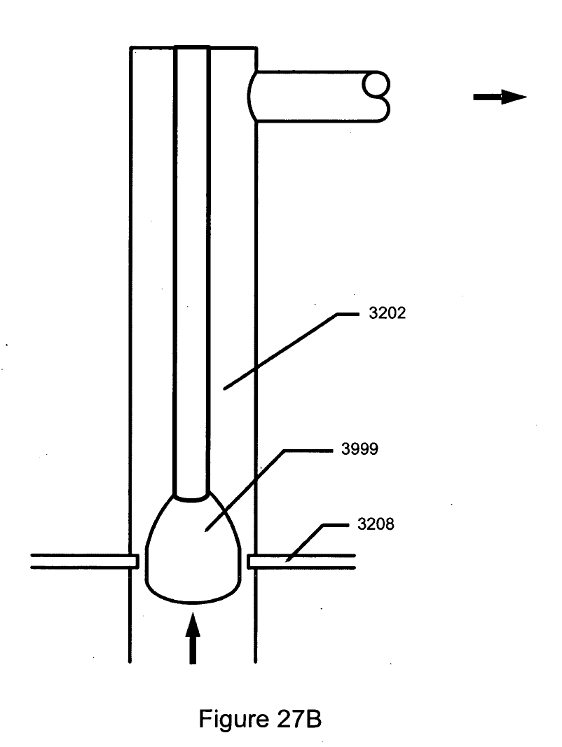

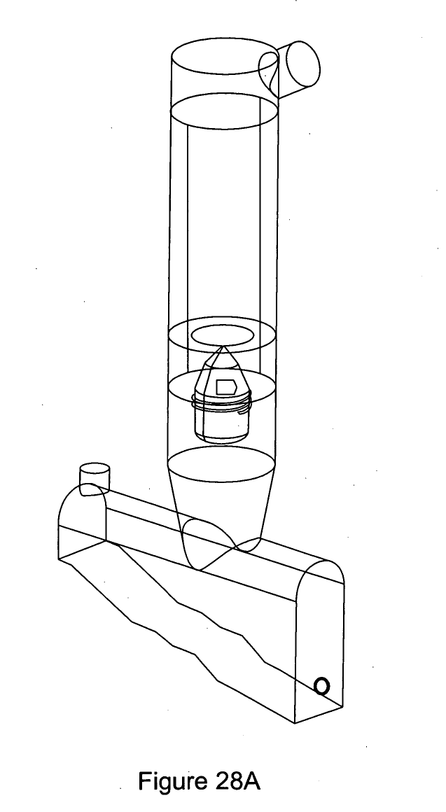

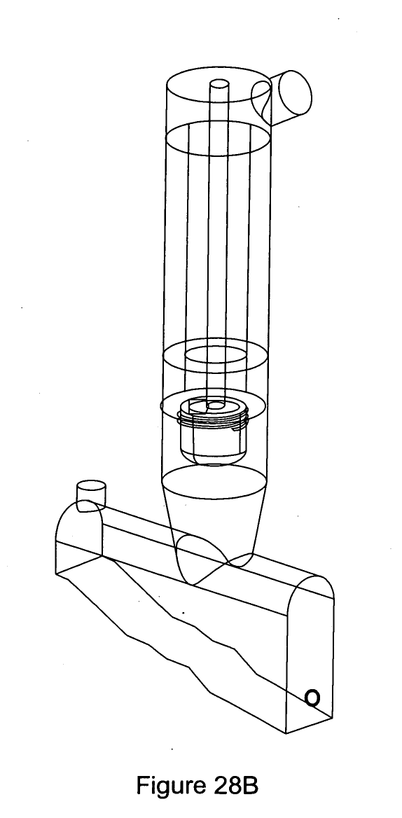

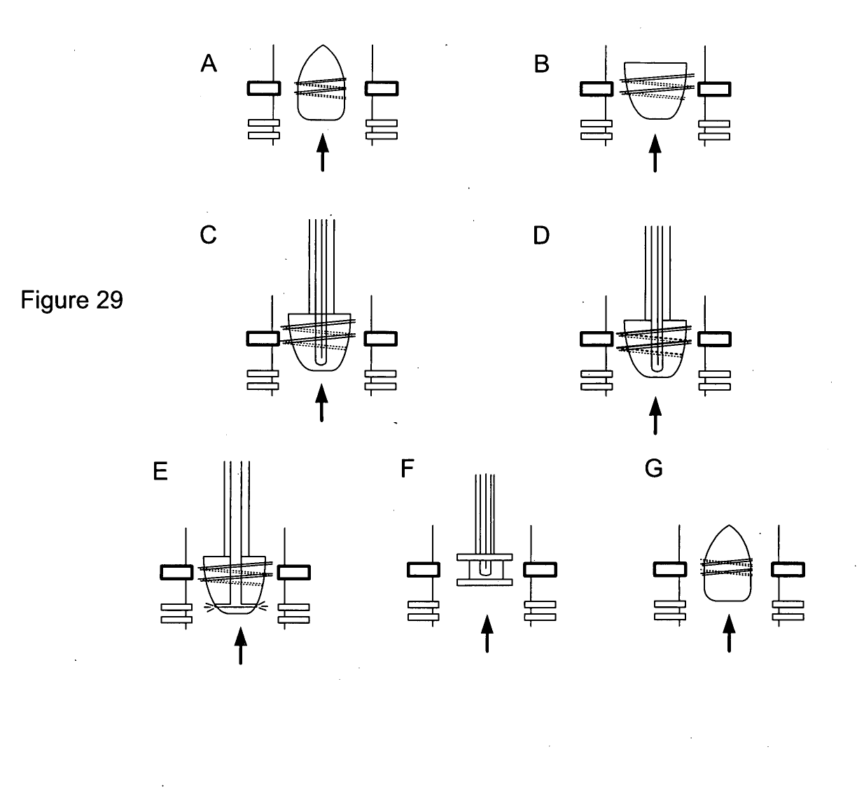

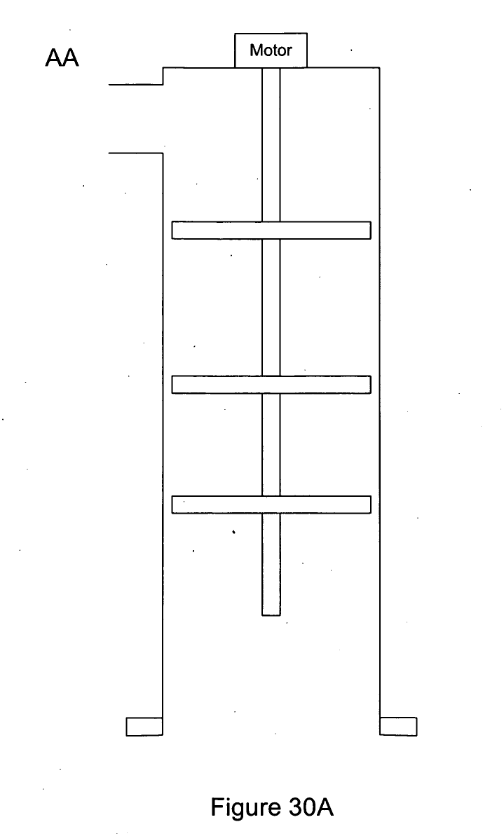

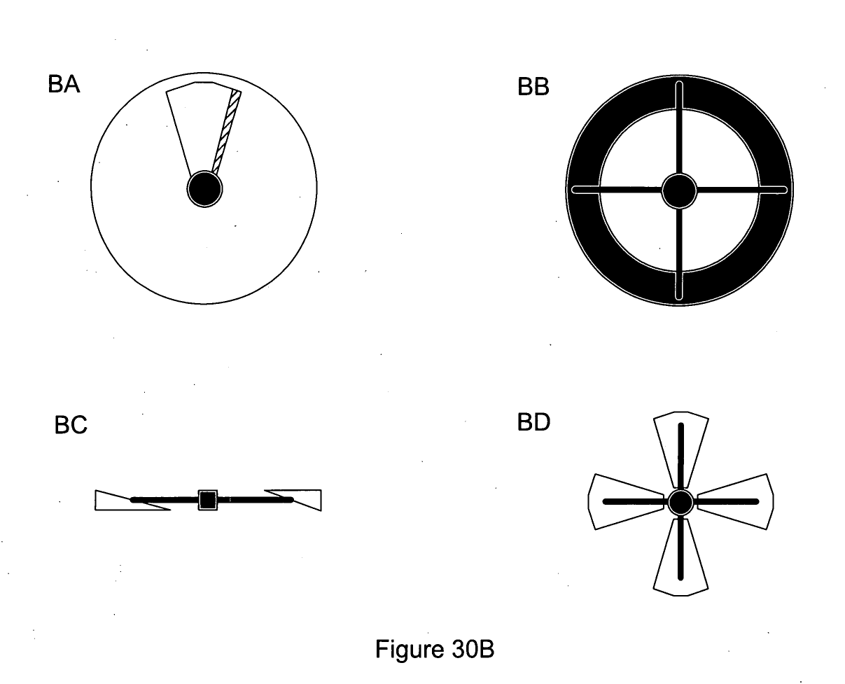

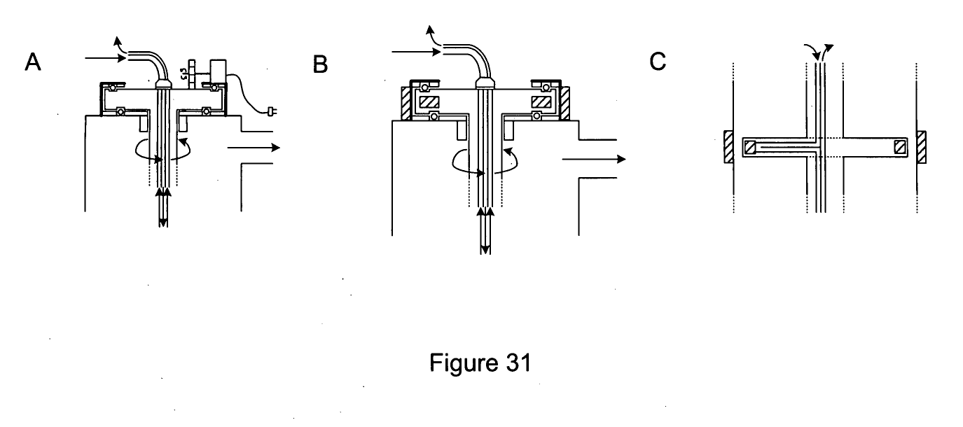

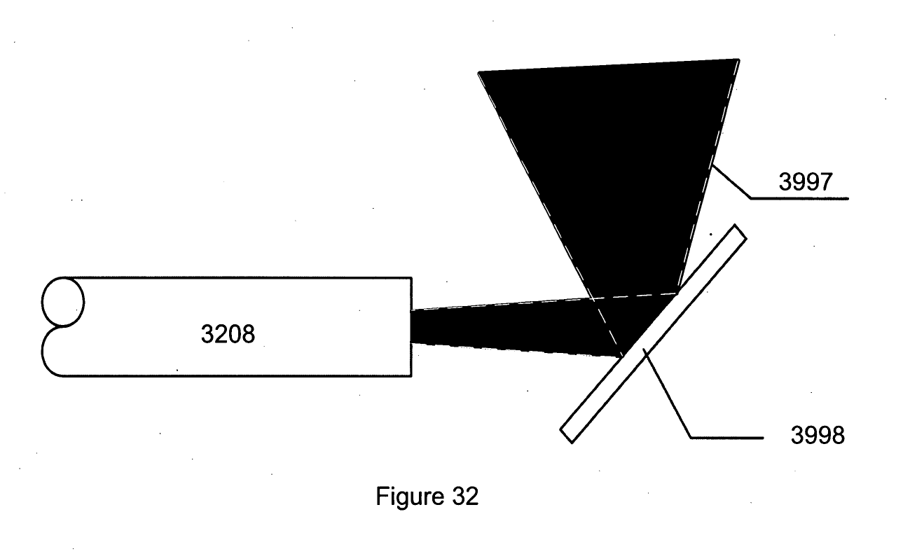

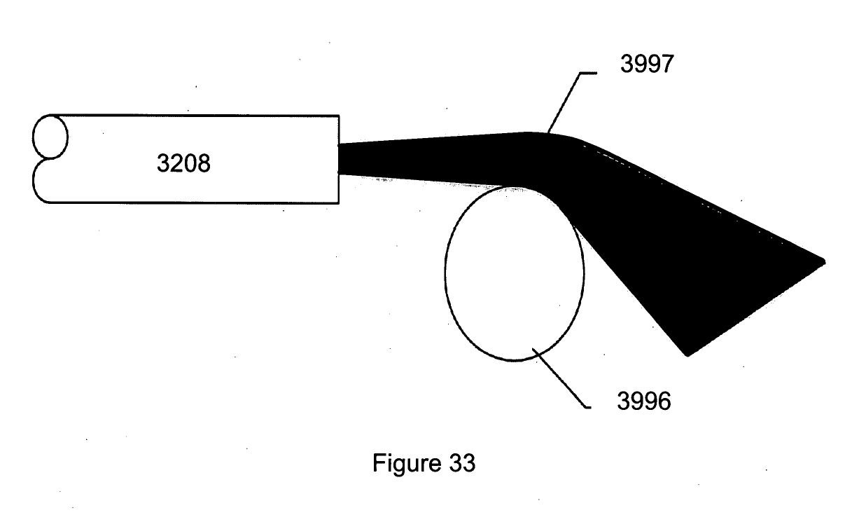

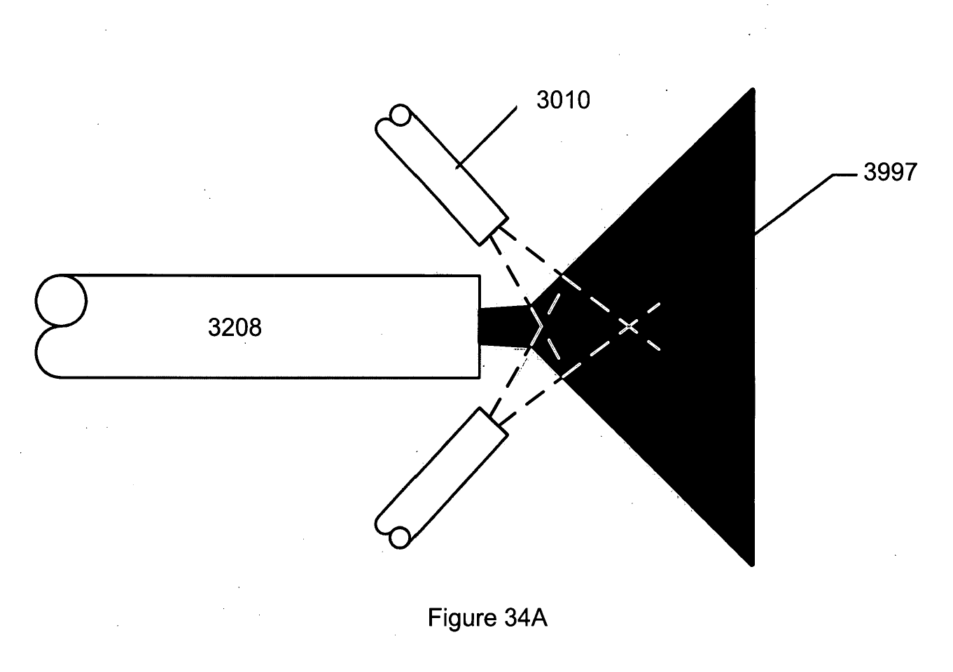

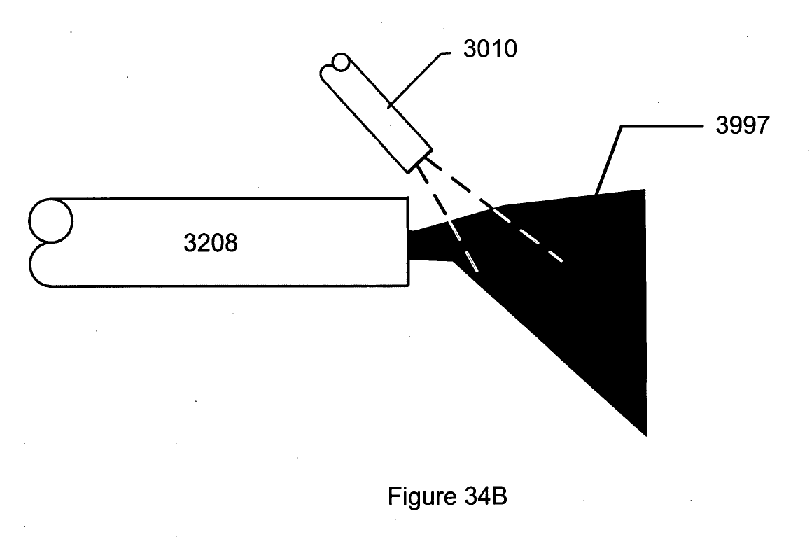

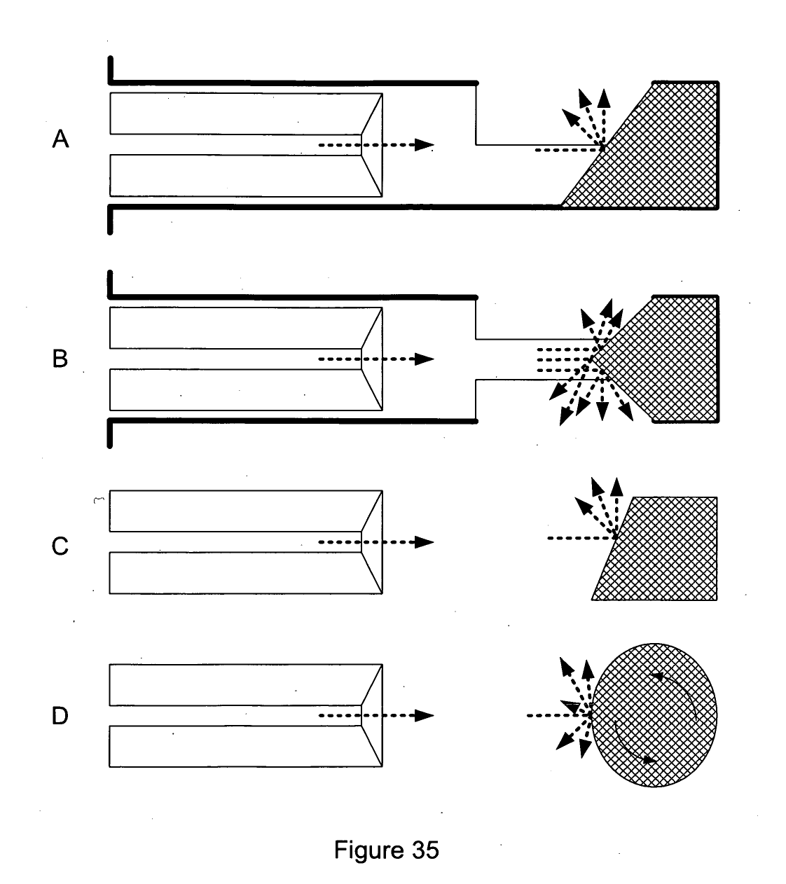

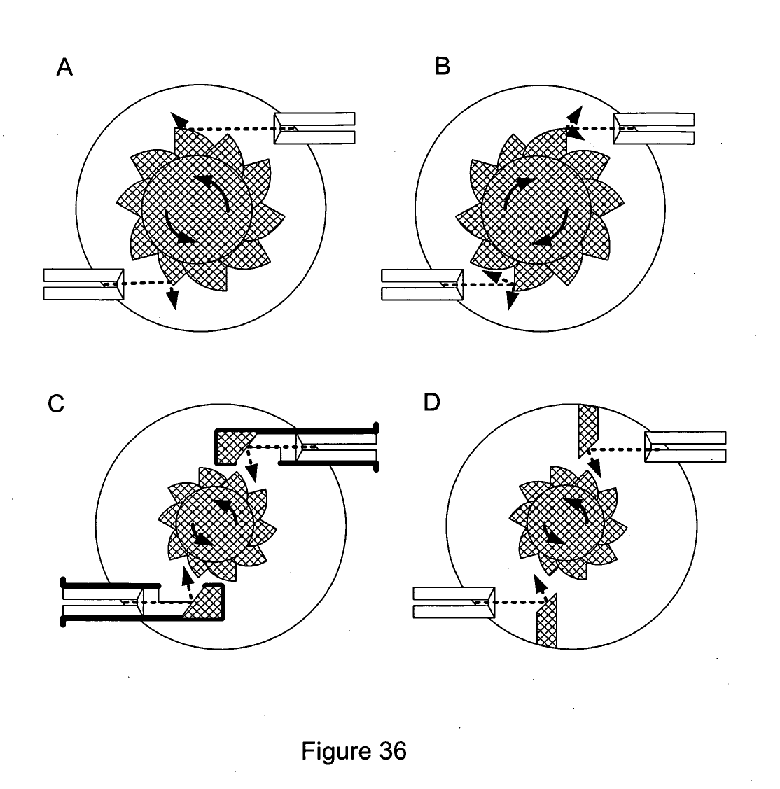

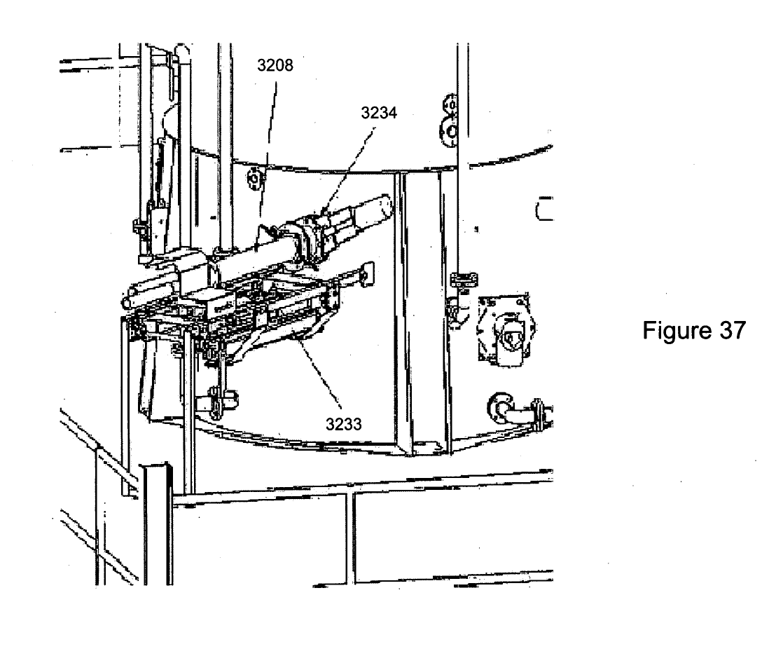

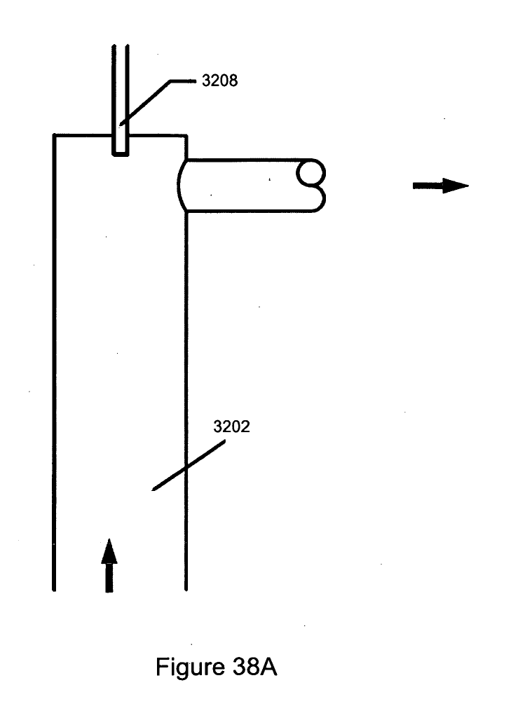

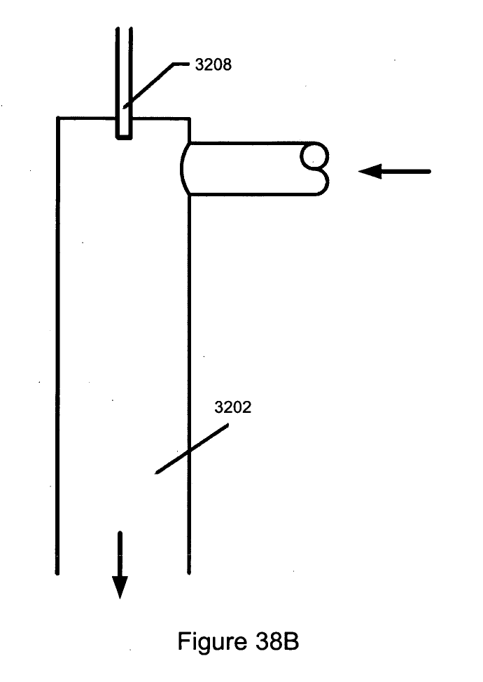

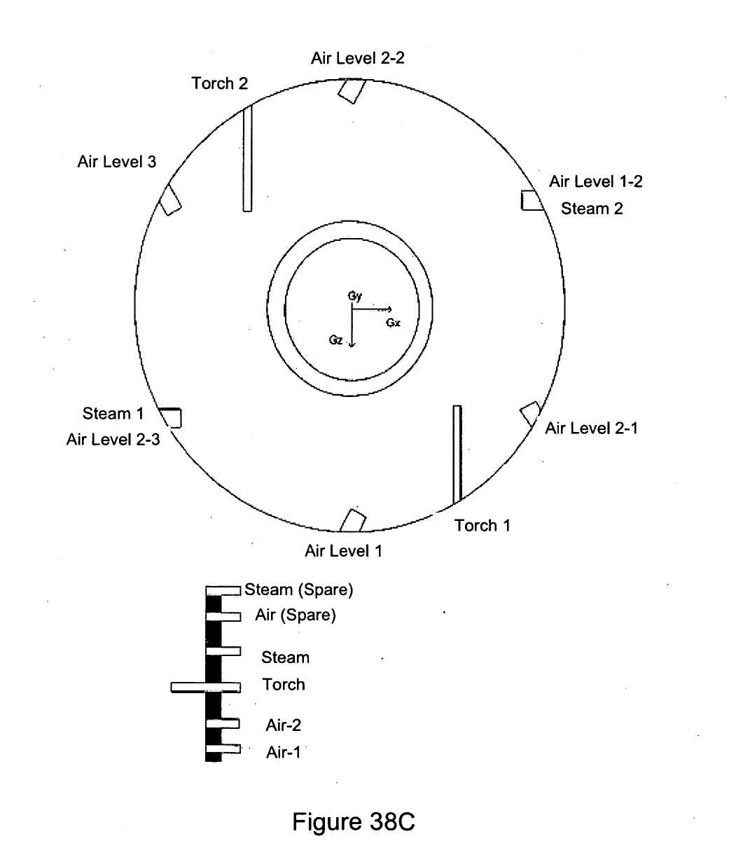

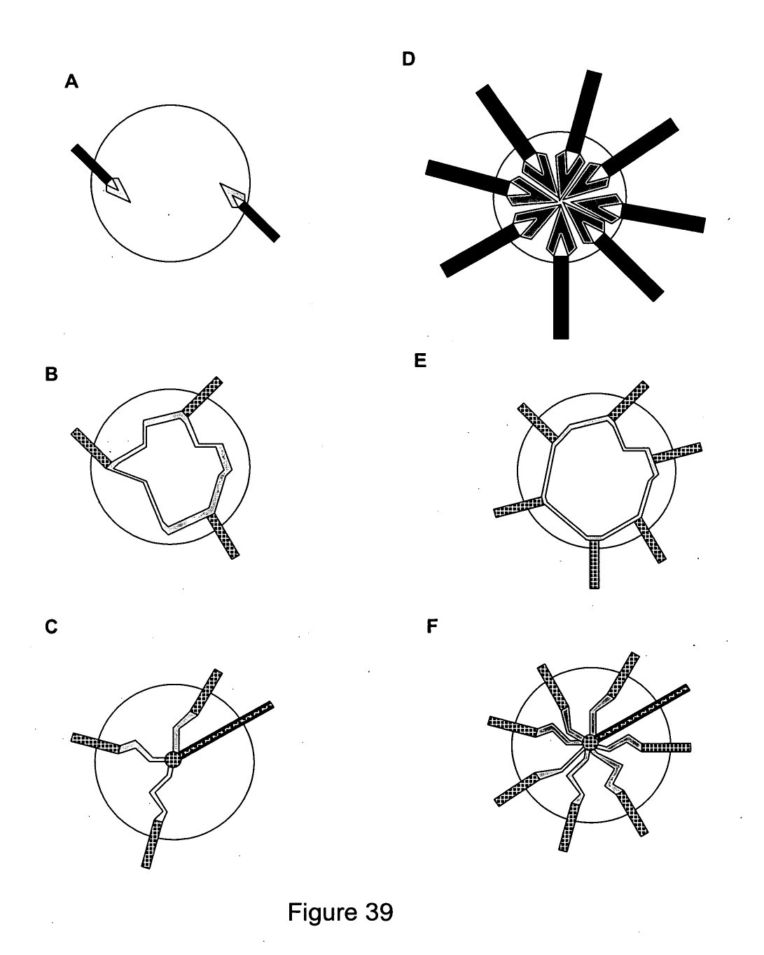

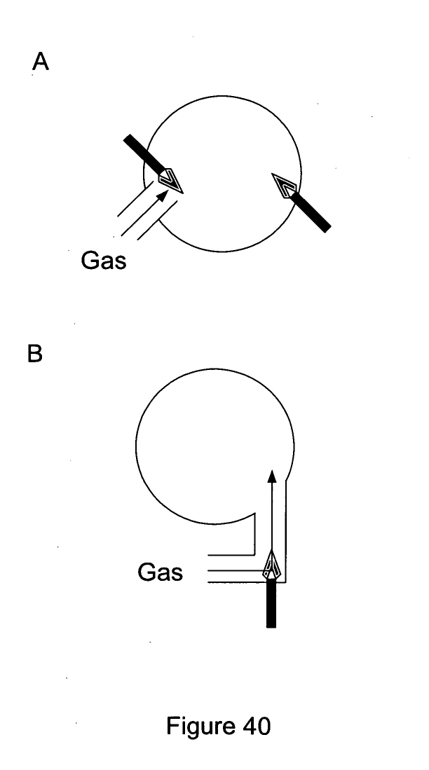

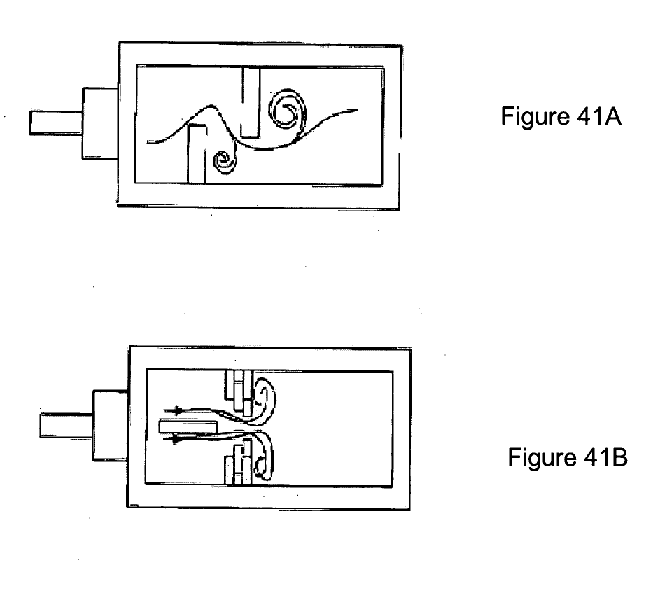

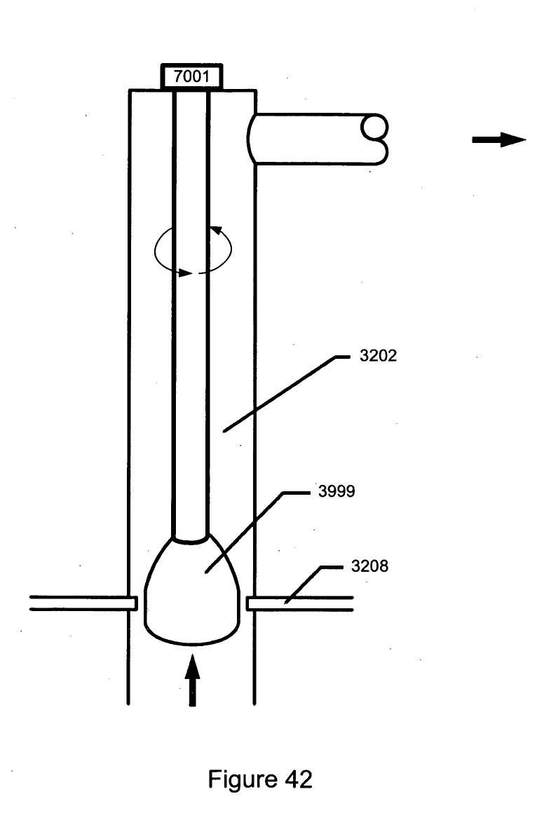

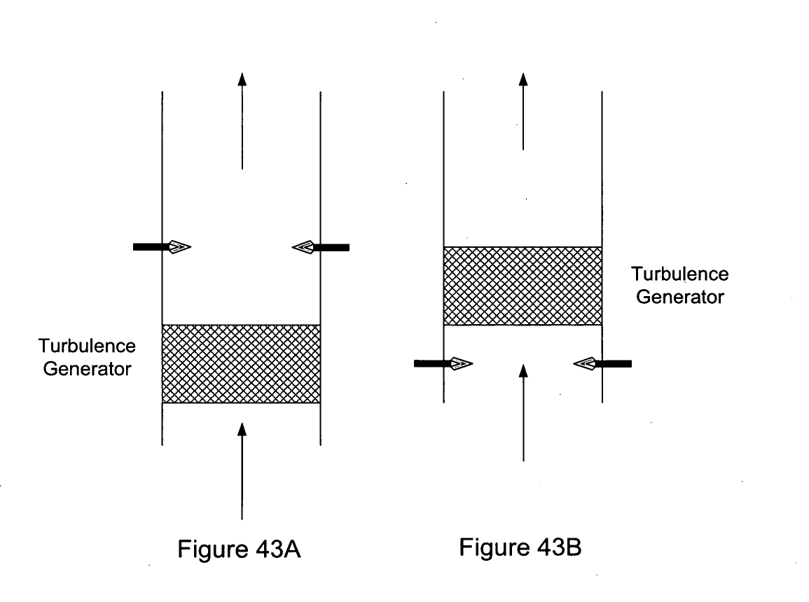

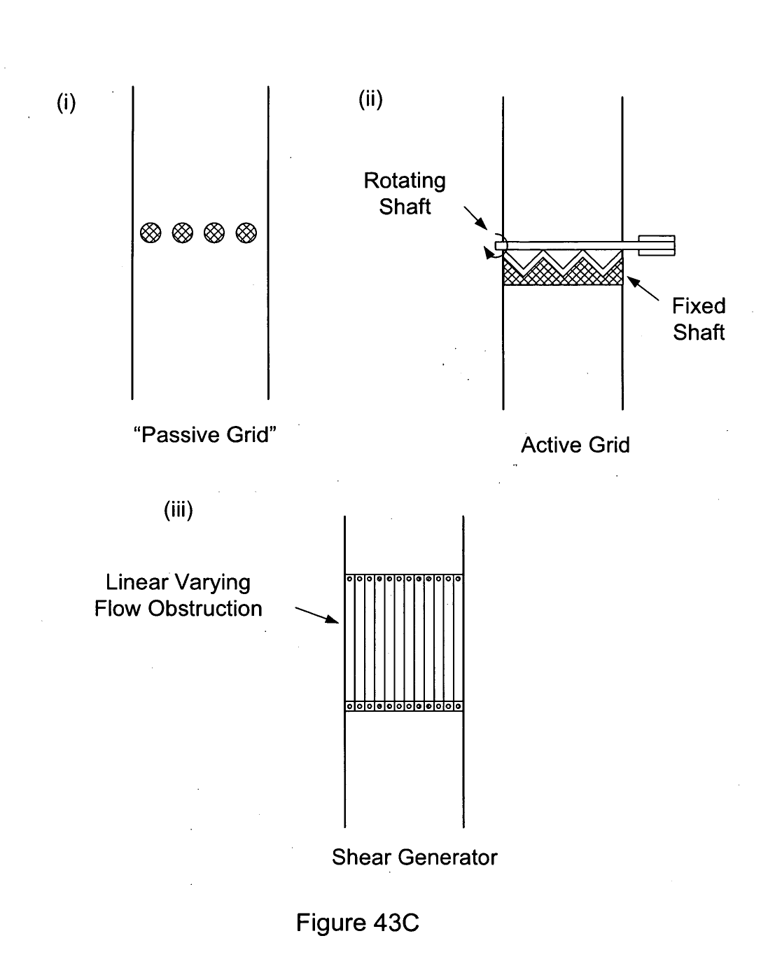

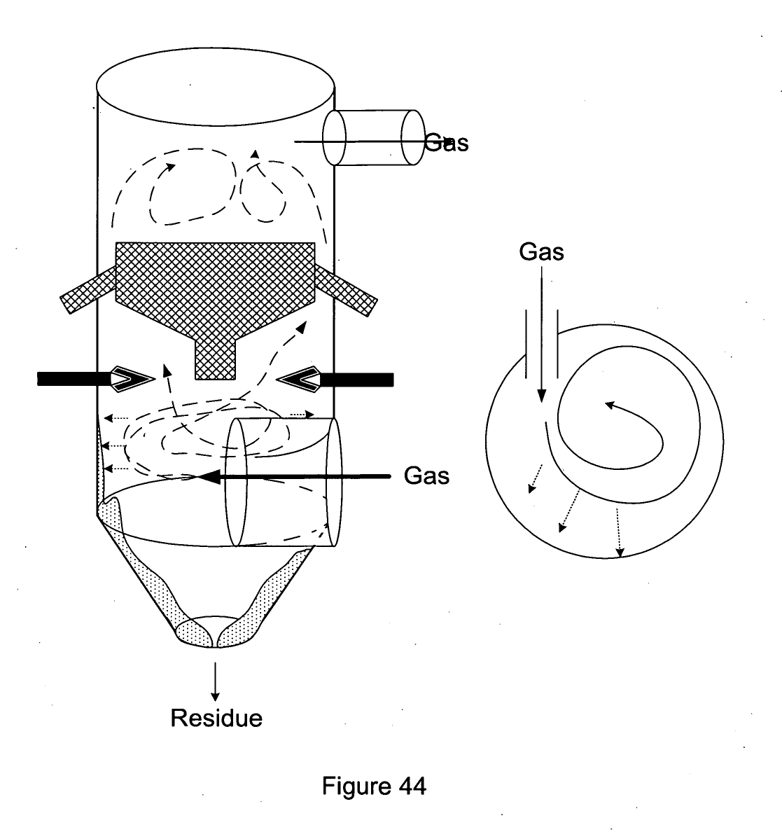

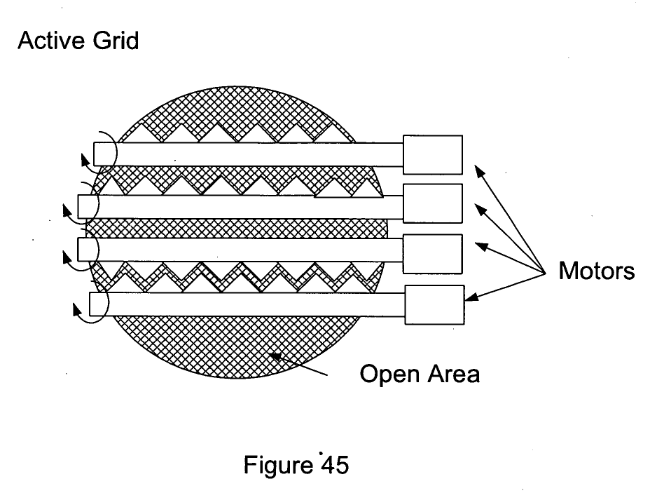

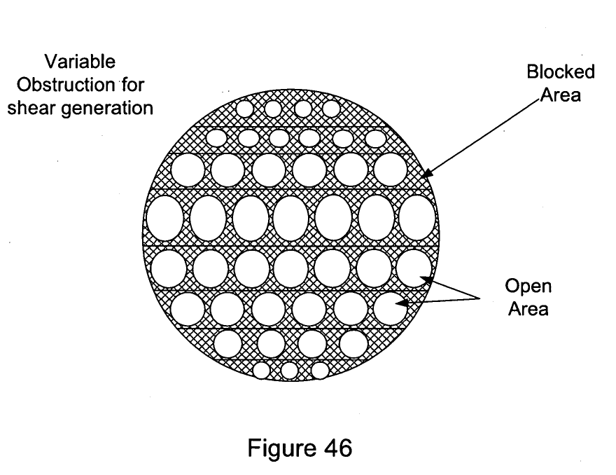

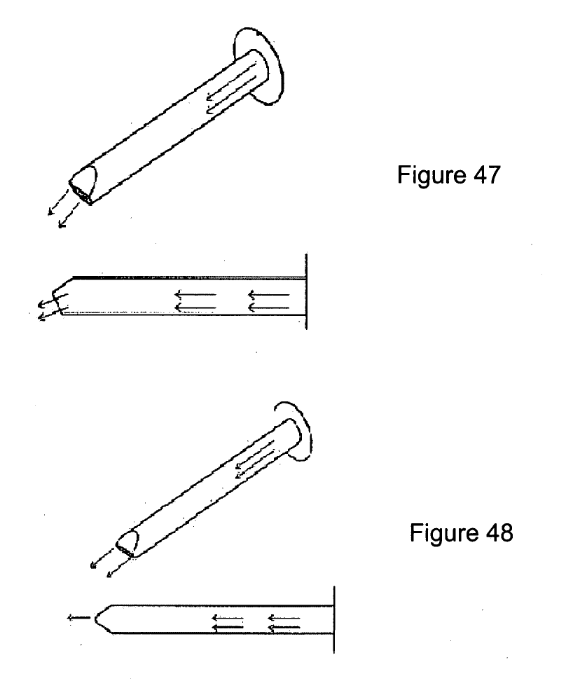

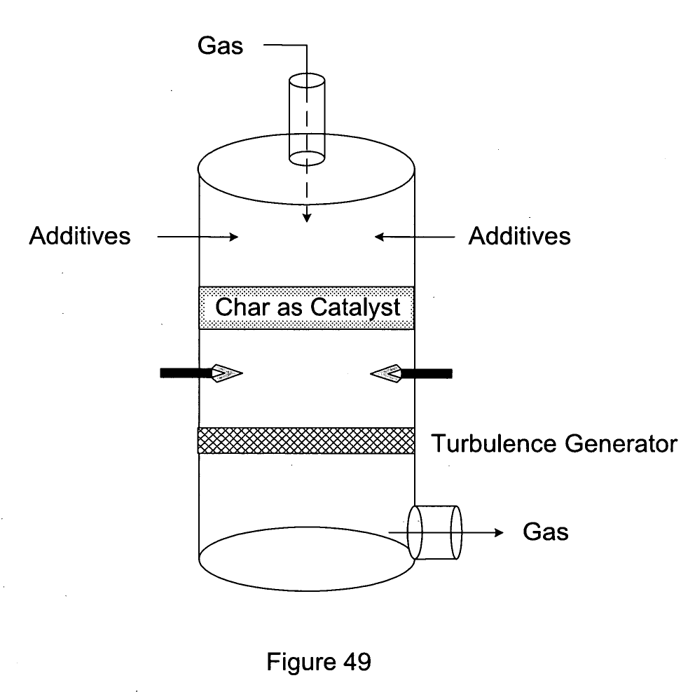

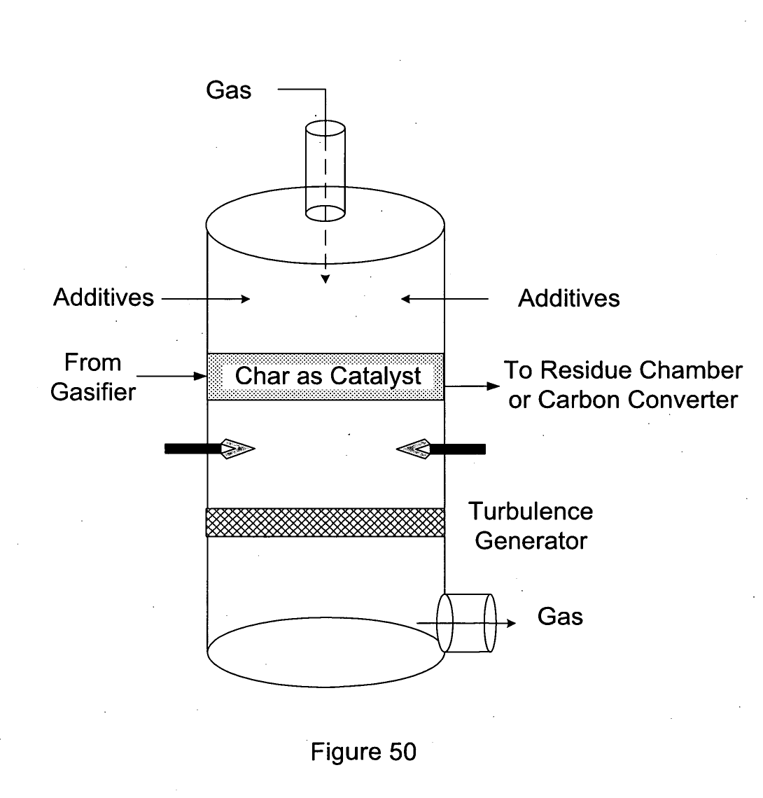

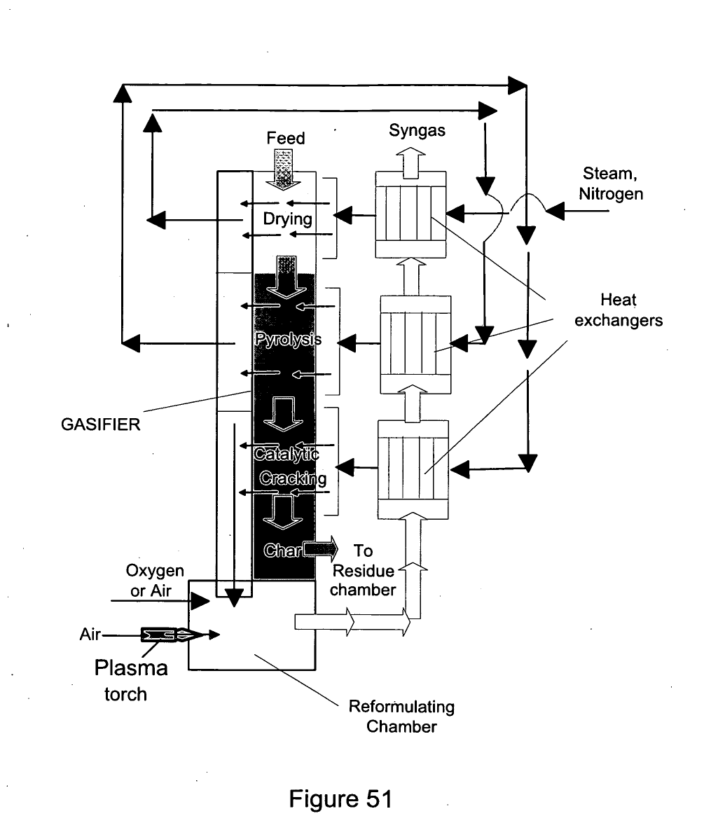

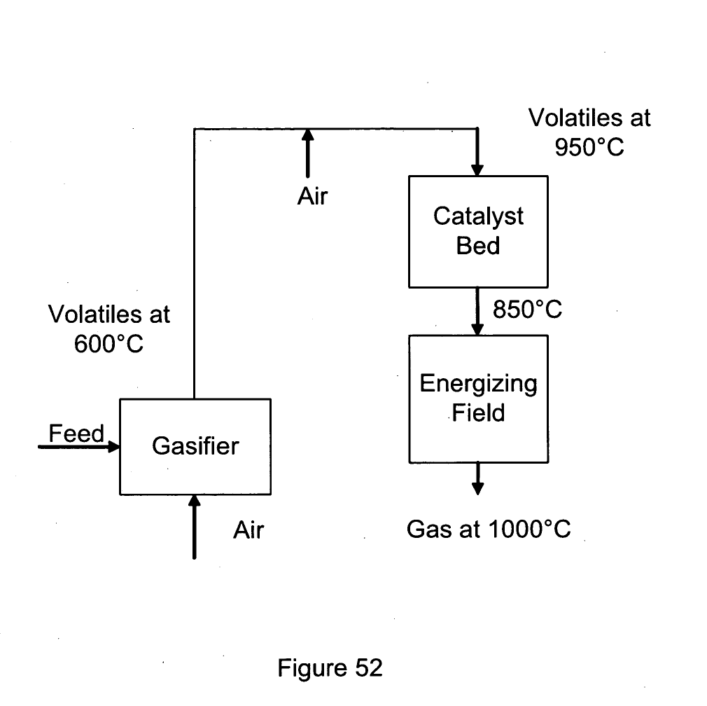

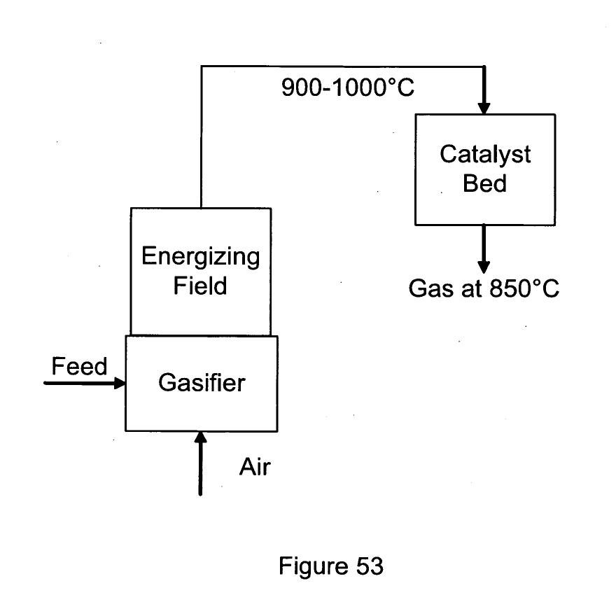

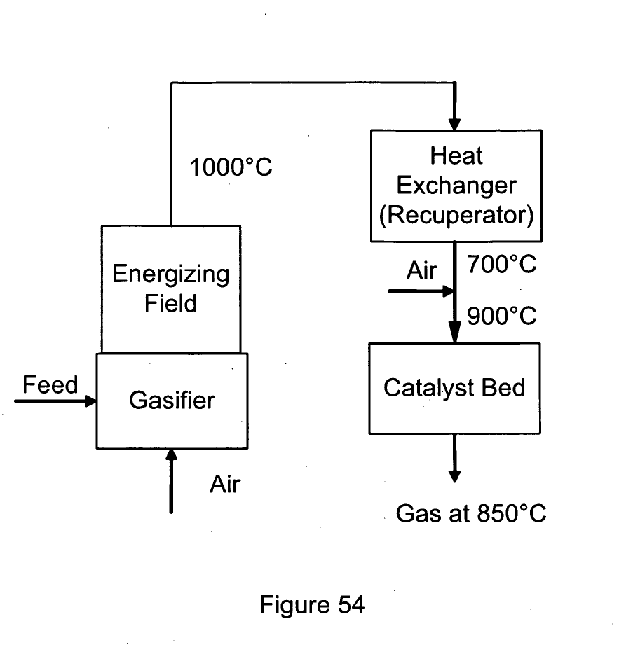

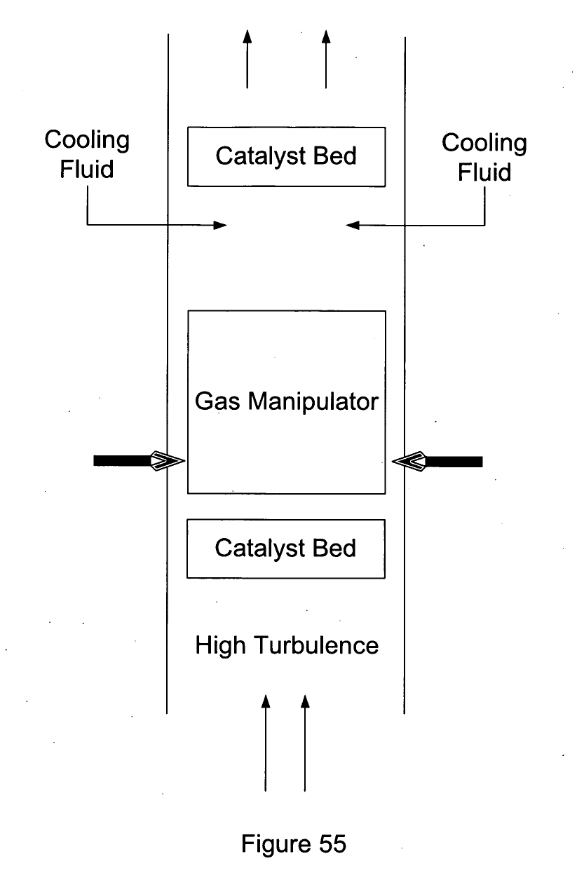

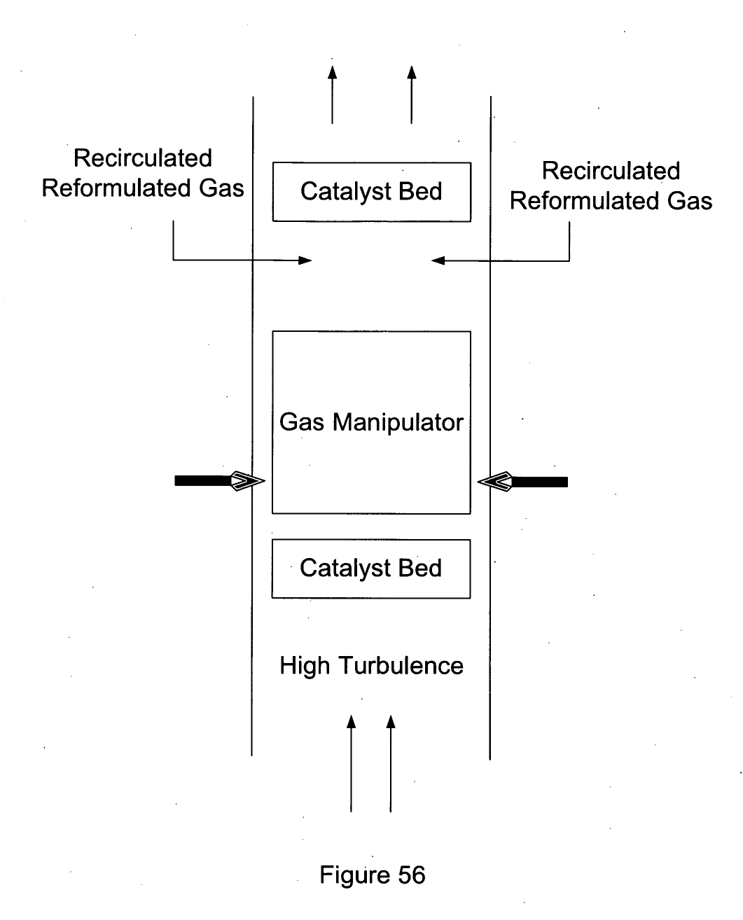

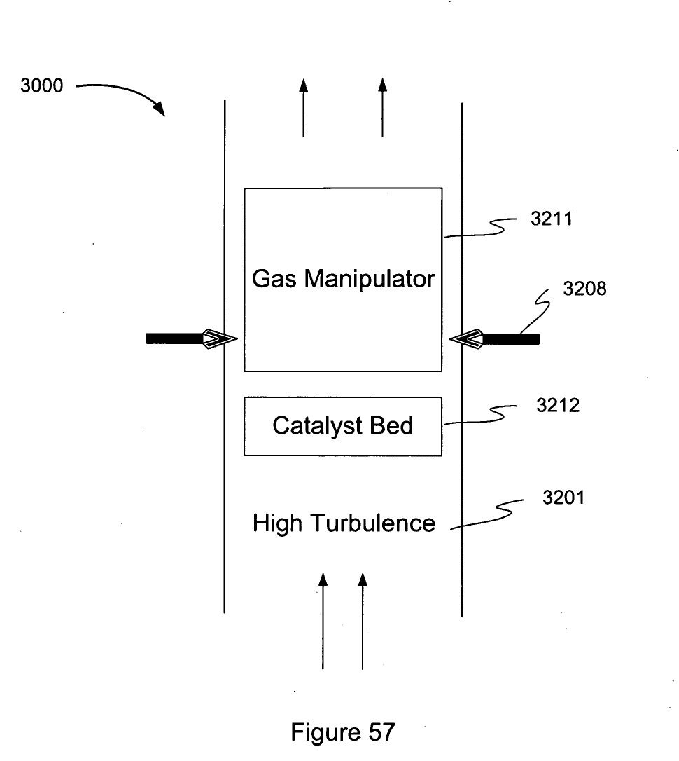

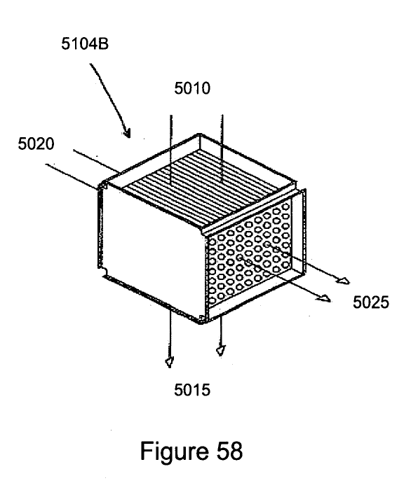

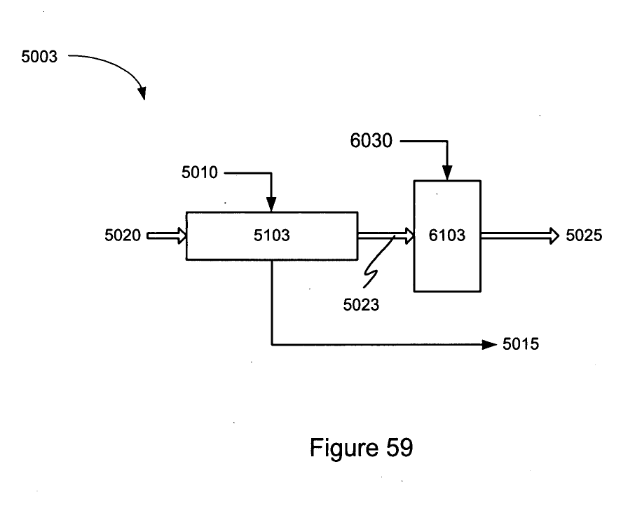

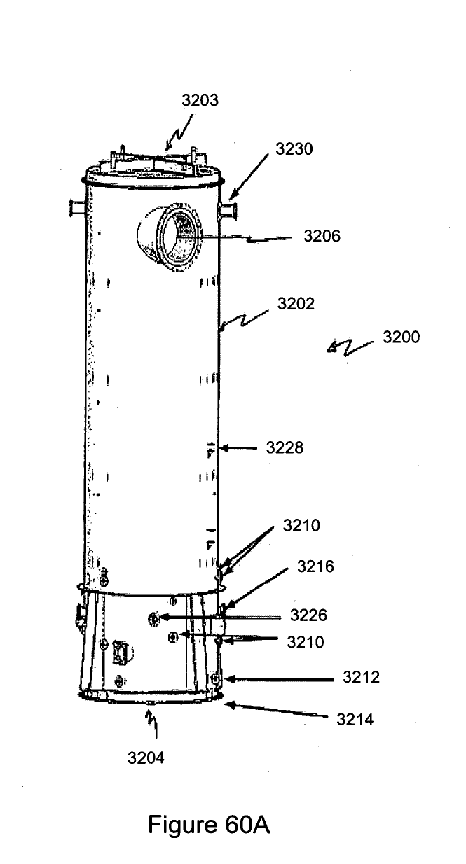

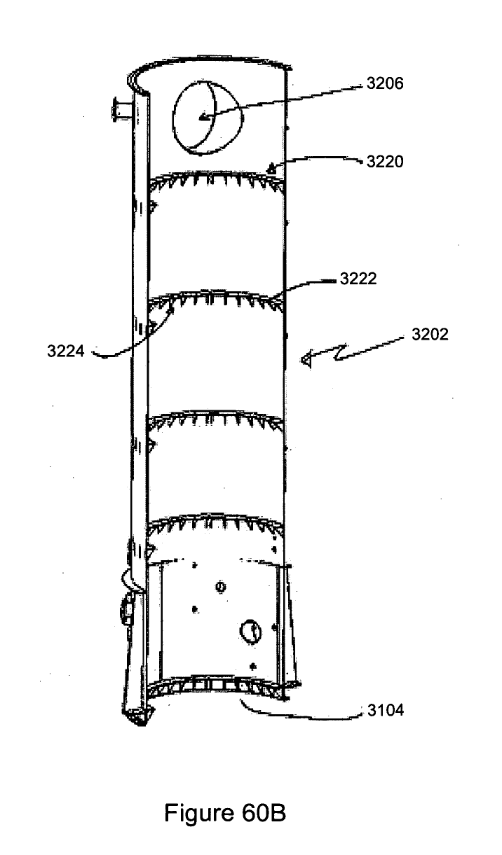

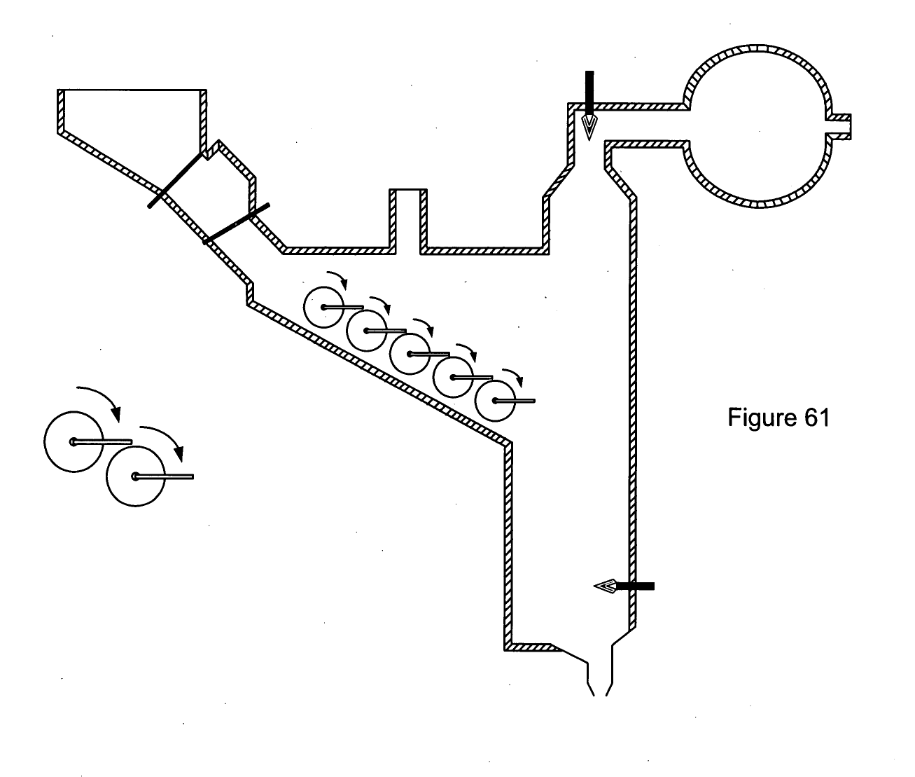

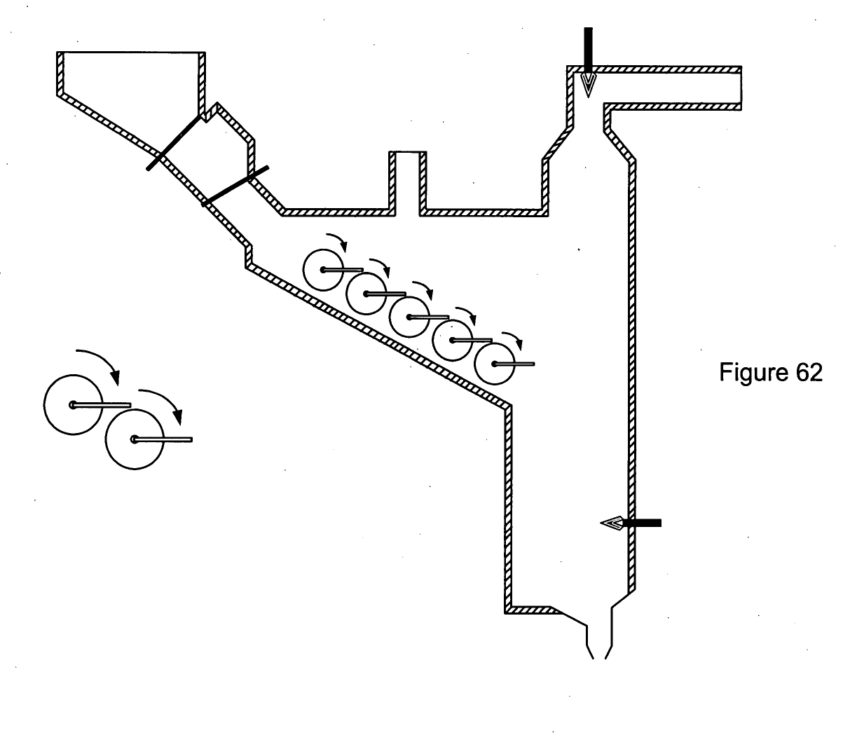

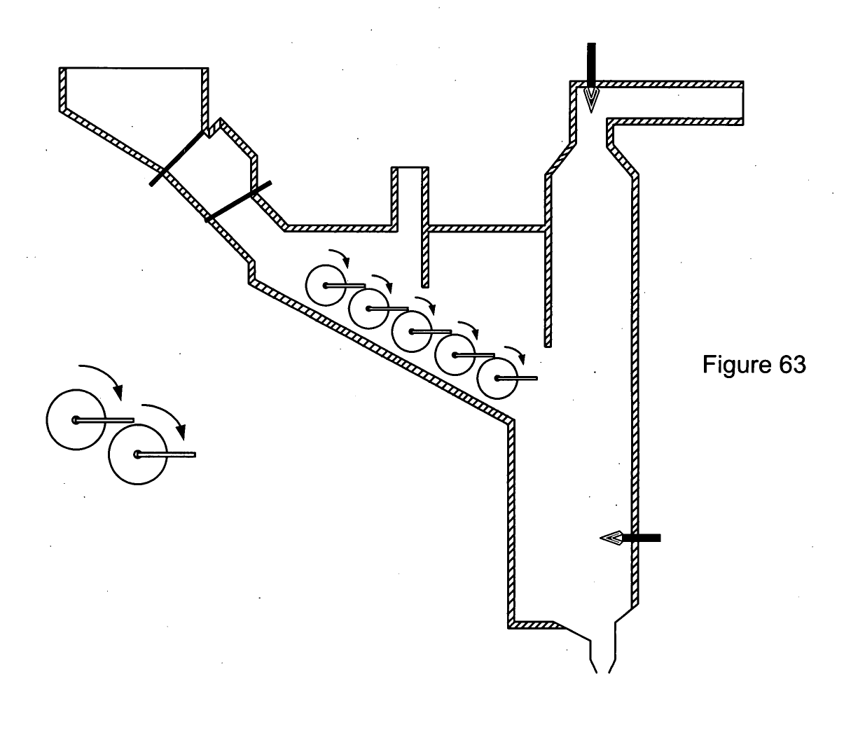

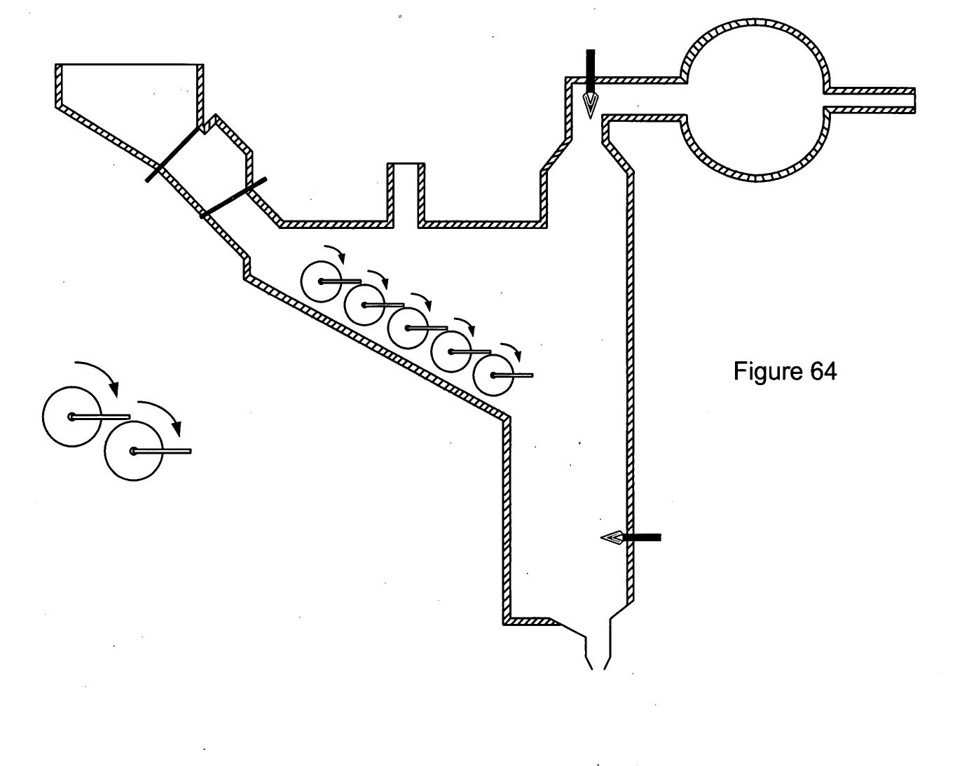

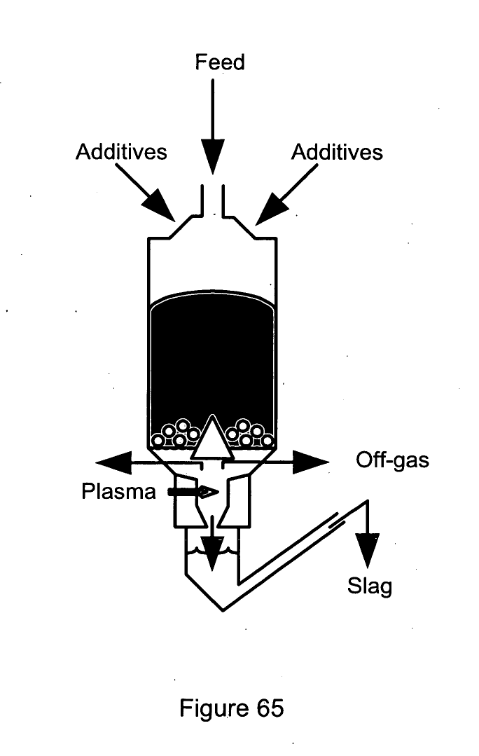

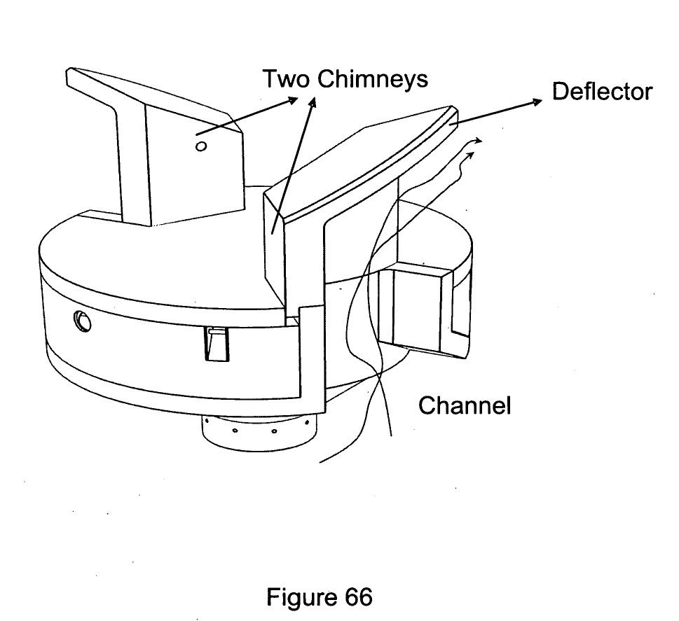

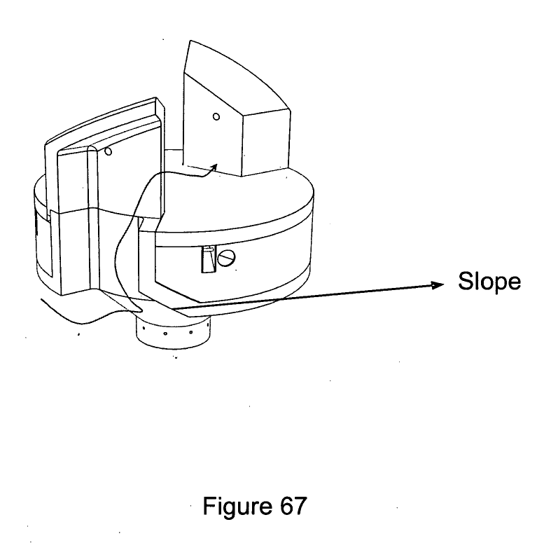

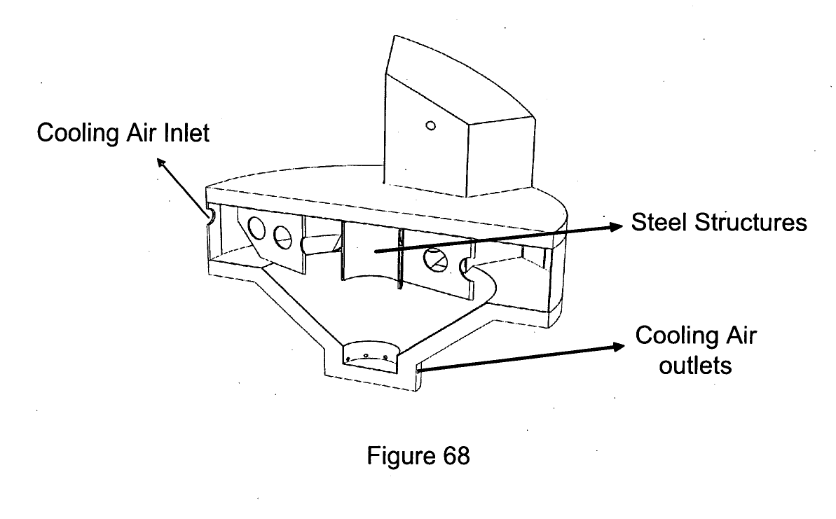

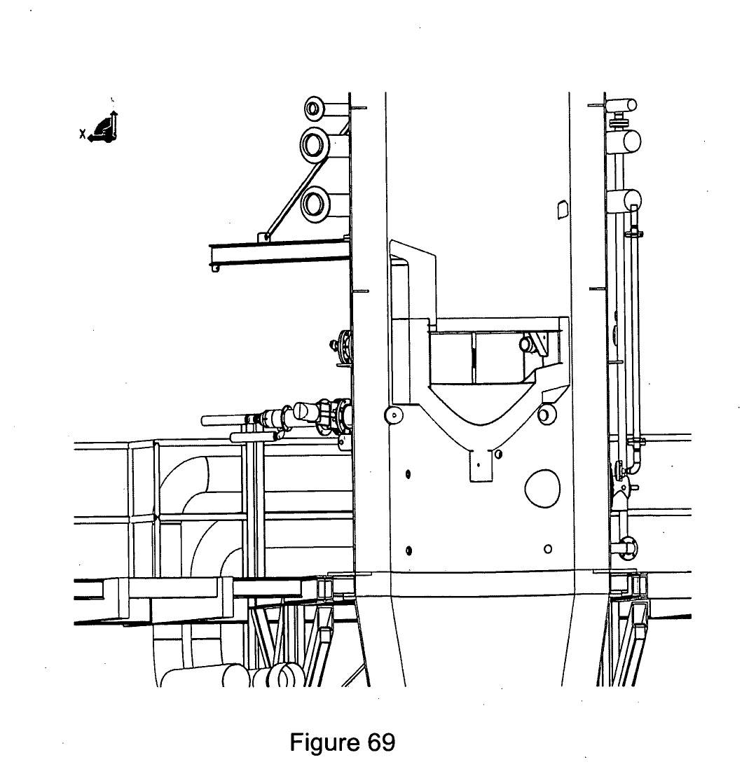

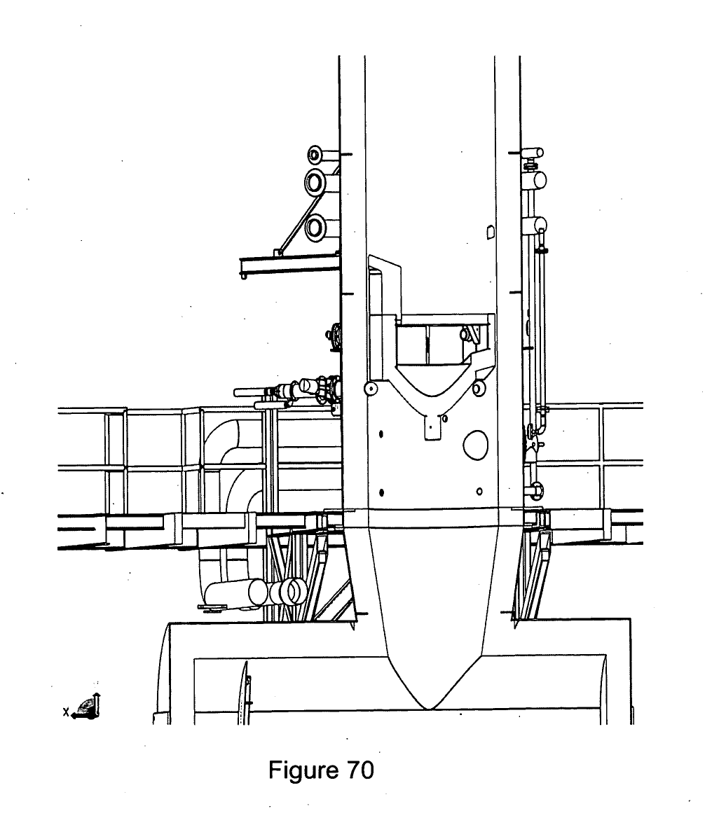

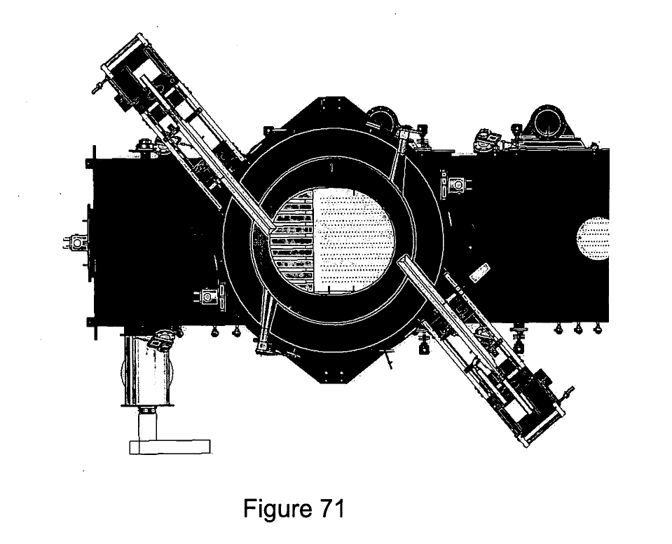

In one embodiment as demonstrated by FIG. 5, the Gas Reformulating System 3000 is a stand-alone unit which receives initial gas from two sources of gas via separate piping or conduits. In one embodiment as shown in FIG. 6, the individual gas streams are combined before they are injected into the Gas Reformulating System 3000. In stand-alone units, the Gas Reformulating System may further comprise appropriate support structures.

An induction blower may be provided downstream of the chamber and in gaseous communication therewith to maintain the pressure of the chamber at a desired pressure, for example a pressure of about 0 to −5 mbar.

The efficacy of the gas reformulation processes occurring within a chamber depends on various factors including but not limited to the chamber internal volume and geometry, gas flow rate, the distance the gas travels and/or the path of the gas through the chamber (i.e., a straight linear passage or a swirling, cyclonic, helical or other non-linear path). The chamber must therefore be shaped and sized to obtain the desired flow dynamics of the gas therein. For example, air jets can be used to promote a swirling flow of the gas through the chamber, such that the passage of the gas is non-linear. Flow modeling of the overall Gas Reformulating System can be used to ensure that a particular chamber design promotes the conditions (e.g. proper interaction of the process inputs) required for the desired gas reformulation.

The one or more chambers of the Gas Reformulating System may be designed in a variety of shapes and be disposed in a variety of positions, as would be readily known to a worker skilled in the art. The chamber can be oriented substantially vertically, substantially horizontally or angularly.

In one embodiment of the invention, the chamber is a straight tubular or venturi shaped structure comprising a first (upstream) end and a second (downstream) end and is oriented in a substantially vertical position or a substantially horizontal position. In one embodiment of the invention, the chamber is a straight cylinder with a length-to-diameter ratio ranging between about 2 to about 6, with associated effects on achievable gas velocities. In one embodiment, the length-to-diameter ratio of the chamber is 3:1.

In one embodiment as depicted in FIG. 60A, the chamber 3202 is configured for direct coupling to a gasifier, and is a straight, substantially vertical, refractory-lined, capped, cylindrical structure with an open bottom (upstream) end 3204 and one reformulated gas outlet 3206 proximal to or at the top (downstream) end of the chamber. The top (downstream) end of the chamber may be capped with a refractory-lined lid 3203, which may be removably sealed to the chamber in order to facilitate maintenance or repair.

The wall of the chamber may be lined with refractory material or otherwise fabricated to withstand high temperatures. The chamber may be encapsulated with a water jacket for cooling and/or generation of steam or recovery of usable torch heat. The chamber may have multiple walls, along with a cooling mechanism for heat recovery, and the gas reformulating system may also include heat exchangers for high pressure/high temperature steam production, or other heat recovery capability.

Conventional refractory materials that are suitable for use in a high temperature, unpressurized chamber are well-known to those skilled in the art and include, but are not limited to, high temperature fired ceramics, i.e., aluminum oxide, aluminum nitride, aluminum silicate boron nitride, zirconium phosphate, glass ceramics and high alumina brick containing principally, silica, alumina, chromia and titania, ceramic blanket and insulating firebrick. Materials such as Didier Didoflo 89CR and Radex Compacflo V253 may be used where a more robust refractory material is required.

In one embodiment, the refractory design has multiple layers with a high density layer on the inside to resist the high temperature, erosion and corrosion present within the chamber and to provide a heat sink to reduce fluctuations in the gas properties. Outside the high density material is a lower density material with lower erosion resistance properties but higher insulation factor. Optionally, outside this layer is a very low density foam board material with very high insulation factor that can be used because it will not be exposed to a corrosive environment which can exist within the chamber. The multilayer design can further optionally comprise an outside layer, between the foam board and the vessel shell that is a ceramic blanket material to provide a compliant layer to allow for differential expansion between the solid refractory and the vessel shell. Appropriate materials for use in a multilayer refractory are well known in the art.

In one embodiment, the multilayer refractory can further comprise segments of compressible refractory separating sections of a non-compressible refractory to allow for expansion of the refractory. The compressible layer can optionally be protected from erosion by overlapping extendible high density refractory. In one embodiment, the multilayer refractory can comprise an internally oriented chromia layer; a middle alumina layer and an outer insulboard layer.

In some embodiments of the invention, the chamber includes a layer of up to about seventeen inches, or more, of specially selected refractory lining throughout the entire chamber to ensure maximum retention of processing heat while being impervious to chemical reaction from the reactive intermediates formed during processing.

The refractory lining in the bottom section of the chamber can be more prone to wear and deterioration since it must withstand higher temperatures from the operating sources of plasma torch heat. In one embodiment, therefore, the refractory in the lower section is designed to comprise a more durable “hot face” refractory than the refractory on the chamber walls and top. For example, the refractory on the walls and top can be made of DIDIER RK30 brick, and the different “hot face” refractory for the lower section can be made with RADEX COMPAC-FLO V253.

In embodiments in which the chamber is refractory-lined, the wall of the chamber can optionally incorporate supports for the refractory lining or refractory anchors.

The chamber may have a collector for solid particulate matter. For embodiments where the chamber is operated in conjunction with a gasifier, any matter that is collected may be fed into a gasifier for further processing or into a solid residue conditioning chamber, for further processing. Collectors for solid particulate matter known in the art include but are not limited to centrifugal separators, inertial impingement baffles and filters. For embodiments where the Gas Reformulating System is directly coupled to the gasifier, additional solid particulate collectors may not be necessary as particulates formed may, in part, fall directly back into the gasifier.

### Ports, Inlets and Outlets for the Chamber

The chamber comprises one or more initial gas inlets that feed the initial gas into the chamber for reformulation, and one or more reformulated gas outlets to pass the reformulated gas further downstream. The inlet may comprise an opening or, alternatively, may comprise a device to control the flow of initial gas into the chamber and/or a device to inject the initial gas into the chamber. The device may include gas manipulators for appropriate injection of the initial gas for enhanced reformulation, and/or include sensing elements for measuring the various characteristics of the initial gas.

The initial gas inlets may be incorporated to promote concurrent, countercurrent, radial, tangential, or other feed flow directions. In one embodiment, the single initial gas inlet has an increasingly conical shape.

The initial gas inlets may be located at or near the first or upstream end of the chamber. In one embodiment, the inlet comprises the open first end of the chamber, whereby it is in direct gaseous communication with the gas source e.g. gasifier. In one embodiment, the inlet comprises an opening located in the closed first (upstream) end of the chamber. In one embodiment, the inlet comprises one or more openings in the wall of the chamber proximal to the first (upstream) end.

In embodiments in which the gasifier and Gas Reformulating System are directly coupled, the attachment site on the gasifier for coupling to the Gas Reformulating System may be strategically located to optimize gas flow and/or maximize mixing of the initial gas prior to entering the chamber. In one embodiment, the chamber is located at the center of the gasifier.

In embodiments in which the chamber is connected to one or more gasifiers, one or more initial gas inlets of the chamber may be in direct communication with the one or more gasifiers through a common opening or as shown in FIG. 5, may be connected to the gasifier via piping 3009 or via appropriate conduits.

The reformulated gas produced in the reformulating reaction exits the chamber through one or more reformulated gas outlets located at or near the second or downstream end. The outlet may comprise an opening or, alternatively, may comprise a device to control the flow of the reformulated gas out of the chamber. The device may include sensing elements for measuring the various characteristics of the reformulated gas.

In one embodiment, the outlet comprises the open second (downstream) end of the chamber. In one embodiment, the outlet comprises one or more openings located in the closed second (downstream) end of the chamber. In one embodiment, the outlet comprises one or more openings in the wall of the chamber near the second (downstream) end.

The chamber optionally comprises various ports including one or more process additive ports, one or more ports for gas energizing sources, optionally one or more access ports, view ports and/or instrumentation ports. Gas energizing sources include but are not limited to plasma-based sources (e.g. plasma torches), hydrogen burners and optional secondary sources. Ports, inlets and outlets may be incorporated at various angles and/or locations to enhance interaction of the reactant flows within the chamber.

### Control System

A control system may be provided to control one or more processes implemented in, and/or by, the various systems and/or subsystems disclosed herein, and/or provide control of one or more process devices contemplated herein for affecting such processes. In general, the control system may operatively control various local and/or regional processes related to a given system, subsystem or component thereof, and/or related to one or more global processes implemented within a larger system, such as a gasification system, within or in cooperation with which the various embodiments of the invention may be operated, and thereby adjusts various control parameters thereof adapted to affect these processes for a defined result. Various sensing elements and response elements may therefore be distributed throughout the controlled system(s), or in relation to one or more components thereof, and used to acquire various process, reactant and/or product characteristics, compare these characteristics to suitable ranges of such characteristics conducive to achieving the desired result, and respond by implementing changes in one or more of the ongoing processes via one or more controllable process devices.

The control system generally comprises, for example, one or more sensing elements for sensing one or more characteristics related to the system(s), process(es) implemented therein, input(s) provided therefor, and/or output(s) generated thereby. One or more computing platforms are communicatively linked to these sensing elements for accessing a characteristic value representative of the sensed characteristic(s), and configured to compare the characteristic value(s) with a predetermined range of such values defined to characterise these characteristics as suitable for selected operational and/or downstream results, and compute one or more process control parameters conducive to maintaining the characteristic value with this predetermined range. A plurality of response elements may thus be operatively linked to one or more process devices operable to affect the system, process, input and/or output and thereby adjust the sensed characteristic, and communicatively linked to the computing platform(s) for accessing the computed process control parameter(s) and operating the process device(s) in accordance therewith.

In one embodiment, the control system provides a feedback, feedforward and/or predictive control of various systems, processes, inputs and/or outputs related to the conversion of carbonaceous feedstock into a gas, so to promote an efficiency of one or more processes implemented in relation thereto. For instance, various process characteristics may be evaluated and controllably adjusted to influence these processes, which may include, but are not limited to, the heating value and/or composition of the feedstock, the characteristics of the product gas (e.g. heating value, temperature, pressure, flow, composition, carbon content, etc.), the degree of variation allowed for such characteristics, and the cost of the inputs versus the value of the outputs. Continuous and/or real-time adjustments to various control parameters, which may include, but are not limited to, heat source power, additive feed rate(s) (e.g. oxygen, oxidants, steam, etc.), feedstock feed rate(s) (e.g. one or more distinct and/or mixed feeds), gas and/or system pressure/flow regulators (e.g. blowers, relief and/or control valves, flares, etc.), and the like, can be executed in a manner whereby one or more process-related characteristics are assessed and optimized according to design and/or downstream specifications.

In systems utilizing pure feed-forward control, changes in the system's environment in the form of a measured disturbance results in a response that is pre-defined, in contrast to a system utilizing feedback control, to maintain a desired state of the system. Therefore, feed-forward control may not have the stability problems of feedback control.

Feed-forward control can be extremely effective when the following prerequisites are met: the disturbance must be measurable, the effect of the disturbance to the output of the system must be known and the time it takes for the disturbance to affect the output must be longer than the time it takes the feed-forward controller to affect the output.

Feed-forward control can respond more quickly to known and measurable kinds of disturbances, but cannot do much with novel disturbances. In contrast, feed-back control deals with any deviation from desired system behavior, but requires the system's measured variable (output) to react to the disturbance in order to notice the deviation.

Feedforward and feedback control are not mutually exclusive; they can be combined so that a quick response can be provided due to the feedforward control, while the feedback system cleans up for any error in the predetermined adjustment made by the feed-forward system.

In one embodiment of the invention, model predictive control techniques may be used.

In corrective, or feedback, control the value of a control parameter or control variable, monitored via an appropriate sensing element, is compared to a specified value or range. A control signal is determined based on the deviation between the two values and provided to a control element in order to reduce the deviation. It will be appreciated that a conventional feedback or responsive control system may further be adapted to comprise an adaptive and/or predictive component, wherein response to a given condition may be tailored in accordance with modeled and/or previously monitored reactions to provide a reactive response to a sensed characteristic while limiting potential overshoots in compensatory action. For instance, acquired and/or historical data provided for a given system configuration may be used cooperatively to adjust a response to a system and/or process characteristic being sensed to be within a given range from an optimal value for which previous responses have been monitored and adjusted to provide a desired result. Such adaptive and/or predictive control schemes are well known in the art, and as such, are not considered to depart from the general scope and nature of the present disclosure.

Alternatively, or in addition thereto, the control system may be configured to monitor operation of the various components of a given system for assuring proper operation, and optionally, for ensuring that the process(es) implemented thereby are within regulatory standards, when such standards apply.

In accordance with one embodiment, the control system may further be used in monitoring and controlling the total energetic impact of a given system. For instance, a given system may be operated such that an energetic impact thereof is reduced, or again minimized, for example, by optimising one or more of the processes implemented thereby, or again by increasing the recuperation of energy (e.g. waste heat) generated by these processes. Alternatively, or in addition thereto, the control system may be configured to adjust a composition and/or other characteristics (e.g. temperature, pressure, flow, etc.) of a product gas generated via the controlled process(es) such that such characteristics are not only suitable for downstream use, but also substantially optimized for efficient and/or optimal use. For example, in an embodiment where the product gas is used for driving a gas engine of a given type for the production of electricity, the characteristics of the product gas may be adjusted such that these characteristics are best matched to optimal input characteristics for such engines.

In one embodiment, the control system may be configured to adjust a given process such that limitations or performance guidelines with regards to reactant and/or product residence times in various components, or with respect to various processes of the overall process are met and/or optimized for. For example, an upstream process rate may be controlled so to substantially match one or more subsequent downstream processes.

In addition, the control system may, in various embodiments, be adapted for the sequential and/or simultaneous control of various aspects of a given process in a continuous and/or real time manner.

In general, the control system may comprise any type of control system architecture suitable for the application at hand. For example, the control system may comprise a substantially centralized control system, a distributed control system, or a combination thereof. A centralized control system will generally comprise a central controller configured to communicate with various local and/or remote sensing devices and response elements configured to respectively sense various characteristics relevant to the controlled process, and respond thereto via one or more controllable process devices adapted to directly or indirectly affect the controlled process. Using a centralized architecture, most computations are implemented centrally via a centralized processor or processors, such that most of the necessary hardware and/or software for implementing control of the process is located in a same location.

A distributed control system will generally comprise two or more distributed controllers which may each communicate with respective sensing and response elements for monitoring local and/or regional characteristics, and respond thereto via local and/or regional process devices configured to affect a local process or sub-process. Communication may also take place between distributed controllers via various network configurations, wherein a characteristics sensed via a first controller may be communicated to a second controller for response thereat, wherein such distal response may have an impact on the characteristic sensed at the first location. For example, a characteristic of a downstream product gas may be sensed by a downstream monitoring device, and adjusted by adjusting a control parameter associated with the converter that is controlled by an upstream controller. In a distributed architecture, control hardware and/or software is also distributed between controllers, wherein a same but modularly configured control scheme may be implemented on each controller, or various cooperative modular control schemes may be implemented on respective controllers.

Alternatively, the control system may be subdivided into separate yet communicatively linked local, regional and/or global control subsystems. Such an architecture could allow a given process, or series of interrelated processes to take place and be controlled locally with minimal interaction with other local control subsystems. A global master control system could then communicate with each respective local control subsystems to direct necessary adjustments to local processes for a global result.

The control system of the present invention may use any of the above architectures, or any other architecture commonly known in the art, which are considered to be within the general scope and nature of the present disclosure. For instance, processes controlled and implemented within the context of the present invention may be controlled in a dedicated local environment, with optional external communication to any central and/or remote control system used for related upstream or downstream processes, when applicable. Alternatively, the control system may comprise a sub-component of a regional an/or global control system designed to cooperatively control a regional and/or global process. For instance, a modular control system may be designed such that control modules interactively control various sub-components of a system, while providing for inter-modular communications as needed for regional and/or global control.

The control system generally comprises one or more central, networked and/or distributed processors, one or more inputs for receiving current sensed characteristics from the various sensing elements, and one or more outputs for communicating new or updated control parameters to the various response elements. The one or more computing platforms of the control system may also comprise one or more local and/or remote computer readable media (e.g. ROM, RAM, removable media, local and/or network access media, etc.) for storing therein various predetermined and/or readjusted control parameters, set or preferred system and process characteristic operating ranges, system monitoring and control software, operational data, and the like. Optionally, the computing platforms may also have access, either directly or via various data storage devices, to process simulation data and/or system parameter optimization and modeling means. Also, the computing platforms may be equipped with one or more optional graphical user interfaces and input peripherals for providing managerial access to the control system (system upgrades, maintenance, modification, adaptation to new system modules and/or equipment, etc.), as well as various optional output peripherals for communicating data and information with external sources (e.g. modem, network connection, printer, etc.).

The processing system and any one of the sub-processing systems can comprise exclusively hardware or any combination of hardware and software. Any of the sub-processing systems can comprise any combination of one or more proportional (P), integral (I) or differential (D) controllers, for example, a P-controller, an I-controller, a PI-controller, a PD controller, a PID controller etc. It will be apparent to a person skilled in the art that the ideal choice of combinations of P, I, and D controllers depends on the dynamics and delay time of the part of the reaction process of the gasification system and the range of operating conditions that the combination is intended to control, and the dynamics and delay time of the combination controller. It will be apparent to a person skilled in the art that these combinations can be implemented in an analog hardwired form which can continuously monitor, via sensing elements, the value of a characteristic and compare it with a specified value to influence a respective control element to make an adequate adjustment, via response elements, to reduce the difference between the observed and the specified value. It will further be apparent to a person skilled in the art that the combinations can be implemented in a mixed digital hardware software environment. Relevant effects of the additionally discretionary sampling, data acquisition, and digital processing are well known to a person skilled in the art. P, I, D combination control can be implemented in feed forward and feedback control schemes.

### Control Elements

Sensing elements contemplated within the present context, as defined and described above, can include, but are not limited to, elements that monitor gas chemical composition, flow rate and temperature of the product gas, monitor temperature, monitor the pressure, monitor opacity of the gas and various parameters relating to the gas energizing source (for example, power and position).

The resulting H₂:CO ratio in the reformulated gas is dependant on various factors not limited to the operating scenario (pyrolytic or with adequate O₂/Air), on the processing temperature, the moisture content and the H₂:CO ratio of the initial gas. Gasification technologies generally yield a product gas whose H₂:CO ratio varies from as high as about 6:1 to as low as about 1:1 with the downstream application dictating the optimal H₂:CO ratio. In one embodiment, the resulting H₂:CO ratio ranges from about 1.1 and about 1.2. In one embodiment, the resulting H₂:CO ratio is 1.1:1.

Taking into account one or more of the above factors, the control system of the invention regulates the composition of the reformulated gas over a range of possible H₂:CO ratios by adjusting the balance between applied gas energizing field (e.g. plasma torch heat), process additives (e.g. air, oxygen, carbon, steam) thereby allowing reformulated gas composition to be optimized for a specific downstream application.

A number of operational parameters may be regularly or continuously monitored to determine whether the Gas Reformulating System is operating within the optimal set point. The parameters being monitored may include, but are not limited to, the chemical composition, flow rate and temperature of the reformulated gas, the temperature at various points within the system, the pressure of the system, and various parameters relating to the gas energizing sources (e.g. power and position of plasma torches) and the data are used to determine if there needs to be an adjustment to the system parameters.

### The Composition and Opacity of the Reformulated Gas

The product gas can be sampled and analyzed using methods well known to the skilled technician. One method that can be used to determine the chemical composition of the product gas is through gas chromatography (GC) analysis. Sample points for these analyses can be located throughout the system. In one embodiment, the gas composition is measured using a Fourier Transform Infrared (FTIR) Analyser, which measures the infrared spectrum of the gas.

A part of this invention is determining whether too much or too little oxygen is present in the reformulated gas stream and adjusting the process accordingly. In one embodiment, an analyzer or sensor in the carbon monoxide stream detects the presence and concentration of carbon dioxide or other suitable reference oxygen rich material. In one embodiment, oxygen is measured directly.

In one embodiment of the invention, a thermogravimetric analyzer (TGA) may be used.

In one embodiment, the sensors analyze the composition of the reformulated gas for carbon monoxide, hydrogen, hydrocarbons and carbon dioxide. Based on the data analyzed, a controller sends a signal to the oxygen and/or steam inlets to control the amount of oxygen and/or steam injected into the chamber and/or a signal to the gas energizing source(s).

In one embodiment, one or more optional opacity monitors are installed within the system to provide real-time feedback of opacity, thereby providing an optional mechanism for automation of process additive input rates, primarily steam, to maintain the level of particulate matter below the maximum allowable concentration.

### The Temperature at Various Locations in System

In an embodiment, there is provided means to monitor the temperature of the reformulated gas and the temperature at sites located throughout the system, wherein such data are acquired on a continuous basis. Means for monitoring the temperature in the chamber, for example, may be located on the outside wall of the chamber, or inside the refractory at the top, middle and bottom of the chamber. Additionally, sensors for monitoring the exit temperature of the reformulated gas are provided.

In an embodiment, the means for monitoring the temperature is provided by thermocouples installed at locations in the system as required.

### The Pressure of System

In one embodiment, there is provided means to monitor the pressure within the chamber, wherein such data are acquired on a continuous, real time basis. In a further embodiment, these pressure monitoring means comprise pressure sensors such as pressure transducers or pressure taps located anywhere on the reaction vessel, for example on a vertical wall of the reaction vessel.

### The Rate of Gas Flow

In an embodiment, there is provided means to monitor the flow rate of reformulated gas at sites located throughout the system, wherein such data are acquired on a continuous basis.

Fluctuations in the gas flow may be the result of non-homogeneous conditions (e.g. torch malfunction or out for electrode change or other support equipment malfunction). As a temporary measure fluctuations in gas flow may be corrected by feedback control of blower speed, feed rates of material, secondary feedstock, air, steam, and torch power. If fluctuations in gas flow persist, the system may be shut down until the problem is solved.

### Addition of Process Additives

In an embodiment, the control system comprises response elements to adjust the reactants, including any process additives, to manage the chemical reformulating of initial gas to reformulated gas. For example, process additives may be fed into the chamber to facilitate the efficient reformulating of an initial gas of a certain chemical composition into a reformulated gas of a different desired chemical composition.

In one embodiment, if the sensors detect excess carbon dioxide in the reformulated gas, the steam and/or oxygen injection is decreased.

Response elements contemplated within the present context, as defined and described above, can include, but are not limited to, various control elements operatively coupled to process-related devices configured to affect a given process by adjustment of a given control parameter related thereto. For instance, process devices operable within the present context via one or more response elements, may include, but are not limited to elements that regulate oxygen source(s) inputs and the gas energizing source(s).

Adjusting Gas Energizing Field (e.g. Power to a Torch)

The gas energizing field may be altered. In one embodiment, the plasma torch heat is controlled to drive the reaction. Addition of air into the chamber also bears part of the torch heat load by releasing torch heat energy with combustion of reformulated gas. The flow rate of process air is adjusted to keep torch power in a suitable operating range.

In one embodiment, the plasma torch power is adjusted to stabilize the reformulated gas exit temperatures at the design set point. In one embodiment, the design set point is above 1000° C. to promote full decomposition of the tars and soot in the gas.

Adjusting Pressure within the System

In one embodiment, the control system comprises a response element for controlling the internal pressure of the chamber. In one embodiment, the internal pressure is maintained at a negative pressure, i.e., a pressure slightly below atmospheric pressure. For example, the pressure of the chamber may be maintained at about 1-3 mbar vacuum. In one embodiment, the pressure of the system is maintained at a positive pressure.

An exemplary embodiment of such means for controlling the internal pressure is provided by an induction blower in gaseous communication with the Gas Reformulating System. The induction blower thus employed maintains the system at a negative pressure. In systems in which positive pressure is maintained the blower is commanded to operate at lower RPM than the negative pressure case or a compressor may be used.

In response to data acquired by pressure sensors located throughout the system, the speed of the induction blower will be adjusted according to whether the pressure in the system is increasing (whereby the fan will increase in speed) or decreasing (whereby the fan will decrease in speed).

Moreover, according to the process of the invention, the system may be maintained under slight negative pressure relative to atmospheric pressure to prevent gases being expelled into the environment.

Pressure can be stabilized by adjusting the reformulated gas blower's speed. Optionally, at speeds below the blower's minimum operating frequency, a secondary control overrides and adjusts the recirculation valve instead. Once the recirculation valve returns to fully closed, the primary control re-engages.

### EXAMPLE 1

This example shows an example of a gas manipulator designed to be retrofitted to an existing gas reformulating chamber design. FIG. 60A shows the gas reformulating system (GRS) 3200 designed to be directly linked to a horizontally oriented, refractory-lined gasifier.

The gas exits through the gas outlet of the gasifier into the GRS 3200 which is sealably coupled to the gasifier via a mounting flange 3214 which directly connects the gasifier gas outlet with the single conically shaped input gas inlet of the GRS. Air is injected into the input gas stream through swirl ports 3212 to create a swirling motion or turbulence in the input gas stream thereby mixing the input gas and creating a re-circulating vortex pattern within the GRS. The residence time of the gas within the GRS is about 1.2 seconds.

Referring to FIG. 60A, the GRS comprises a substantially vertically mounted refractory-lined cylindrical chamber having a length-to-diameter ratio of about 3:1 and a single conically shaped input gas inlet to which the gasifier is connected to via a mounting flange 3214. The chamber is capped with a refractory-lined lid 3203 thereby creating a sealed gas reformulating chamber 3202.

The gas reformulating chamber comprises various ports including one or more ports for heaters 3216, one or more ports for one or more oxygen sources 3210, and optionally one or more access or view ports 3326 and/or instrumentation ports 3226. In addition, the gas reformulating chamber is equipped with lifting points 3230.

The refractory used on the wall of the chamber is a multilayer design with a high density layer on the inside to resist the high temperature, erosion and corrosion that is present in the chamber, a middle lower density material layer with lower resistance properties but higher insulation factor and an outer very low density foam board layer with very high insulation factor. The outside layer, between the foam board and the vessel steel shell is a ceramic blanket material to provide a compliant layer to allow for differential expansion between the solid refractory and the vessel shell. Vertical expansion of the refractory is provided for by means of a compressible refractory layer separating sections of the non-compressible refractory. The compressible layer is protected from erosion by overlapping but extendible high density refractory.

Referring to FIG. 60B, the gas reformulating chamber further comprises a refractory support system comprising a series of circumferential extending shelves 3220. Each shelf is segmented and includes gaps to allow for expansion. Each shelf segment 3222 is supported by a series of support brackets 3224.

In this embodiment of the GRS, the one or more inputs for one or more oxygen source(s) include air and steam inputs.

The GRS further comprises three levels of tangentially positioned air nozzles, two tangentially located plasma torches, six thermocouple ports, two burner ports, two pressure transmitter ports and several spare ports.

Air is injected into the gas stream by three levels of air nozzles that include four jets at the lower level 3212 and another six jets at upper level 3211 in which three jets are slightly higher than other three to create cross-jet mixing effects to achieve better mixing.

The GRS further includes two-tangentially mounted 300 kW, water cooled, copper electrode, NTAT, DC plasma torches mounted on a sliding mechanism. The two plasma torches are located above the air nozzles to provide maximum exposure to plasma torch heat.

The plasma power supply converts three phase AC power into DC power for each plasma torch. As an intermediate step, the unit first converts the three phase AC input into a single high frequency phase. This allows for better linearization of the eventual DC output in the chopper section. The unit allows output DC voltage is allowed to fluctuate in order to maintain stable DC current.

Referring to FIG. 37, each plasma torch 3208 is mounted on a sliding mechanism that can move the torch 3208 into and out of the gas reformulating chamber. The torch 3208 is sealed to the gas reformulating chamber 3202 by means of a sealing gland. This gland is sealed against a gate valve, which is, in turn, mounted on and sealed to the vessel. To remove a torch 3208, it is pulled out of the reformulating chamber 3202 by the slide mechanism. Initial movement of the slide disables the high voltage torch power supply for safety purposes. The gate valve shuts automatically when the torch 3208 has retracted past the valve and the coolant circulation is stopped. The hoses and cable are disconnected from the torch 3208, the gland is released from the gate valve and the torch 3208 is lifted away by a hoist.

Replacement of a torch 3208 is done using the reverse of the above procedure; the slide mechanism can be adjusted to permit variation of the insertion depth of the torch 3208. The gate valve is operated mechanically so that operation is automatic. A pneumatic actuator 3233 is used to automatically withdraw the torch in the event of cooling system failure. Compressed air for operating the actuator is supplied from a dedicated air reservoir so that power is always available even in the event of electrical power failure. The same air reservoir provides the air for the gate valve 3234. An electrically interlocked cover is used a further safety feature by preventing access to the high voltage torch connections.

Thermocouples are positioned at various locations with the gas reformulating chamber such that the temperature of the reformulated gas within the GRS is maintained at about 1000° C. and if it falls below this temperature power to the plasma torches or air injection is increased.

In this embodiment, the air flows into the GRS may be varied dynamically to adjust temperatures and processes occurring within each step of the gasifier and/or GRS

The molecules within the gaseous mixture within the gas reformulating chamber disassociate into their constituent elements in the plasma arc zone and then reformed into reformulated gas. The hot reformulated gas exits the GRS via the reformulated gas outlet 3206.

The gas manipulator was designed to enhance the gas reformulation process and achieve the maximum decomposition rate of large hydrocarbon molecules by improving the exposure of the preformulated gas to the reactive species created by the plasma torches and the mixing of the reactive intermediates generated by such exposure.

Referring to FIGS. 69 and 70, the gas manipulator is substantially located in the center of the gas reformulating chamber, and above the air nozzles and the two plasma torches. Thus, the initial gases received from the gasifier are mixed with the air introduced through the air nozzles at high injection velocity.

The shape of the gas manipulator is shown in FIGS. 66 to 68. The preformulated gas obtained by mixing of the initial gas from the gasifier and the injected air, along with the ionized gas of the plasma torches are forced by the design of the gas manipulator to pass through its two channels. As the plasma torches are located substantially at the entrance of the channels, the preformulated gas undergoes maximal exposure to the gas energizing field created by the plasma torches.

The temperature of the gases inside the channel of the gas manipulator is about 1100° C. The gas passing through the channels changes flow directions as it hits the deflectors shown in FIG. 66, resulting in continuous mixing. The deflector also helps to maintain the heat inside the gas manipulator channels thus allowing enhanced gas reformulation kinetics.

Referring to FIG. 67, the sloped surface at the entrance of the gas manipulator enhances the separation of the particulate matter from the gas stream.

The gas manipulator is made of refractory lined steel structure, as shown in FIG. 68. The steel structure is cooled by air. The cooling air is introduced through the three supporting pipes. It passes though the internal empty chamber, cooling the steel structure. The heated cooling air goes back to the main process through the nozzles at the bottom of gas manipulator chamber.

The cooling air flow rate is controlled to maintain the hottest steel surface possible (close to the chimney) but still less than 550° C., at which temperature, the strength of steel is fairly good.

The invention being thus described, it will be apparent that the same may be varied in many ways. Such variations are not to be regarded as a departure from the spirit and scope of the invention, and all such modifications as would be apparent to one skilled in the art are intended to be included within the scope of the following claims.

The embodiments of the invention in which an exclusive property or privilege is claimed are defined as follows:

1) A system for reformulating an initial gas into a reformulated gas having designed characteristics, comprising:
a) a means for sensing at least one characteristic of the initial gas;
b) means for modifying process inputs for reformulation based on the at least one characteristic of the initial gas, and on the designed characteristics of the reformulated gas;
c) means for applying one or more energy sources sufficient to reformulate a substantial majority of the gaseous molecules of the initial gas into the reformulated gas;
d) means for promoting the reformulation;
e) means for stabilizing the reformulated gas; and
f) a control system.

2) The system of claim 1 wherein the means for modifying process inputs comprises means for adding appropriate amounts of process additives.

3) A process for reformulating an initial gas into a reformulated gas having desired characteristics, comprising one or more of the following steps:
a) sensing at least one characteristic of the initial gas;
b) modifying process inputs for reformulation based on the sensed characteristics of the initial gas, and on the desired characteristics of the output gas;
c) applying a gas energizing field sufficient to reformulate the majority of the gaseous molecules into their constituents;
d) promoting efficient process acceleration for the reformulation of the constituents into a reformulated gas of designed characteristics;
e) promoting the de-energization and stabilization of the newly formed molecules to maintain the designed characteristics; and
f) managing the efficient conversion of the initial gas to the output gas.

4) The process of claim 3 wherein the step of modifying process inputs for reformulation comprises adding appropriate amounts of process additives.

5) A system for the reformulation of gas comprising:
a) one or more energy sources for the initiation of gas reformulation processes; and
b) one or more Gas Manipulators for the optimization of energy transference throughout the process of gas reformulation;
wherein the one or more energy sources and the one or more Gas manipulators are integrated to optimize the Gas Reformulation Ratio.

6) A gas reformulation system comprising:
a) one or more gas reformulating zones;
b) one or more gas stabilizing zones;
c) comprises a control system that regulates the overall process.
d) optionally one or more gas additive zones, and/or
e) optionally one or more gas cleaning zones,
wherein the zones of the system are arranged and controlled in such a manner that the majority of the initial gas is reformulated into gas of a designed composition.

7. A method for reformulating of an initial gas into a reformulated gas, comprising the steps of:
(a) delivering the initial gas to a gas reformulating chamber;
(b) mixing the input gas with at least one process additive to create preformulated gas;
(c) exposing the preformulated gas to a gas energizing field thereby disassociating molecules within the gas into their constituent elements;
(d) reforming the constituent elements into molecular species of a designed chemical composition and thereby producing the reformulated gas; and
(e) removing the reformulated gas from the chamber.

8. The method according to claim 7, wherein the gas energizing field is created by one or more plasma torches.

9. The method according to claim 7, further comprising the step exposing the reformulated gas to a gas stabilization zone prior to removing the reformulated gas from the chamber.

10. The method according to 7, wherein the reformulation is enhanced by a gas manipulator.

11. A system for reformulating of an initial gas into a reformulated gas comprising:
one or more refractory-lined chambers comprising:
one or more inputs for receiving the initial gas;
one or more outputs for releasing the reformulated gas;
one or more process additive inputs in fluid communication with the chamber;
one or more gas manipulators located in the one or more chambers;
means to create a gas energizing field within the one or more chambers.

12. The system according to claim 11, wherein the means to create a gas energizing field is one or more plasma torches.

13. The system according to claim 11 or 12, wherein the one or more gas manipulators increase turbulence within the chamber.

14. The system according to claim 11 or 12, wherein the one or more gas manipulators alter the flow dynamics with the chamber.

15. The system according to claim 11 or 12, wherein the one or more gas manipulators improve exposure of the preformulated gas to the gas energizing field.
